(19) United States
(12) Patent Application Publication
(10) Pub. No.: US 2013/0338152 A1
(43) Pub. Date: Dec. 19, 2013

(54) FLUOROPHENYL BICYCLIC HETEROARYL COMPOUNDS

(71) Applicants: Bei Chen, San Diego, CA (US); Robin Alec Fairhurst, Riehen (CH); Songchun Jiang, San Diego, CA (US); Wenshuo Lu, San Diego, CA (US); Thomas H. Marsilje, III, San Diego, CA (US); Clive McCarthy, Childrey (GB); Pierre-Yves Michellys, San Marcos, CA (US); Stefan Stutz, Basel (CH)

(72) Inventors: Bei Chen, San Diego, CA (US); Robin Alec Fairhurst, Riehen (CH); Songchun Jiang, San Diego, CA (US); Wenshuo Lu, San Diego, CA (US); Thomas H. Marsilje, III, San Diego, CA (US); Clive McCarthy, Childrey (GB); Pierre-Yves Michellys, San Marcos, CA (US); Stefan Stutz, Basel (CH)

(73) Assignees: IRM LLC, Hamilton HM LX (BM); NOVARTIS AG, Basel (CH)

(21) Appl. No.: 14/003,474

(22) PCT Filed: Mar. 8, 2012
(86) PCT No.: PCT/IB12/51088

§ 371 Date: Sep. 6, 2013

Related U.S. Application Data
(60) Provisional application No. 61/450,417, filed on Mar. 8, 2011.

(51) Int. Cl. C07D 487/04; A61K 31/519; A61K 45/06; A61K 31/541

(52) U.S. Cl. CPC C07D 487/04; A61K 31/541; A61K 31/519; A61K 45/06
USPC 514/224.2; 544/58.2; 544/280; 544/47; 514/228.5; 514/252.16; 514/265.1

(57) ABSTRACT

The present invention provides a compound the invention, and its use as a IGF-1R inhibitor. The present invention further provides a combination of pharmacologically active agents and a pharmaceutical composition.

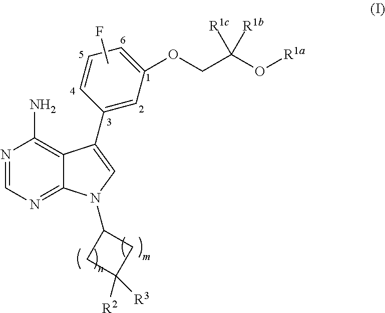

The invention relates to new fluorophenyl bicyclic heteroaryl compounds, processes for the preparation of such compounds; pharmaceutical compositions comprising such compounds optionally in combination with one or more other pharmaceutically active compounds, such compounds optionally in combination with one or more other pharmaceutically active compounds, such compounds optionally in combination with one or more other pharmaceutically active compounds for the treatment of a disease or disorder such as a proliferative disease, especially a tumour disease (also including a method for the treatment of such diseases in mammals, especially in humans), and the use of such derivatives for the preparation of a pharmaceutical composition (medicament) for the treatment of a disease or disorder, in particular a proliferative disease such as a tumour.

Insulin-like growth factor (IGF-1) signaling is typically implicated in cancer, with the IGF-1 receptor (IGF-1R) as the predominating factor. IGR-1R is typically important for tumor transformation and survival of malignant cells, but is typically only partially involved in normal cell growth. Targeting of IGF-1R has been suggested to be a promising option for cancer therapy. (Larsson et al., Br. J. Cancer 92:2097-2101 (2005)).

WO 2005/097800 discloses certain 6,6-bicyclic ring substituted heterobicyclic derivatives having therapeutic activity as IGF-1R inhibitors. WO 2005/037836 discloses certain imidazopyrazine derivatives having therapeutic activity as IGF-1R inhibitors. WO 97/028161 discloses certain pyrrolopyrimidine derivatives having therapeutic activity as inhibitors of tyrosine proteine kinase. WO 2002/092599 discloses certain pyrrolopyrimidine derivatives having therapeutic activity as IGF-1R inhibitors. Mulvihill et al. (Bioorg. Med. Chem. Lett. 17 (2007) 1091 ff) disclose certain imidazopyrazines as IGF-1R inhibitors.

Because of the emerging disease-related roles of IGF-1R, there is a continuing need for compounds which may be useful for treating and preventing a disease which responds to inhibition of IGF-1R.

There is a need to provide new inhibitors of the tyrosine kinase activity of the Insulin-like growth factor I receptor (IGF-IR) that are good drug candidates. Compounds of the invention bind potently to the IGF-1R receptor, and show low affinity for other receptors. Particular compounds of the invention possess favourable pharmacokinetic properties, are non-toxic and demonstrate few side-effects. Furthermore, the ideal drug candidate will exist in a physical form that is stable, non-hygroscopic and easily formulated.

The compounds of the invention are selective IGF-1R inhibitors. In particular, they show an affinity for the IGF-1R which is greater than their affinity for other kinase receptors.

The compounds of the present invention are therefore potentially useful in the treatment of a wide range of disorders, particularly cancer, or other diseases and disorders such as acute lung injury and pulmonary fibrosis and diabetic retinopathy.

The treatment of cancer is a contemplated use. All forms of cancer are potentially treatable with the compounds of the present invention including the specific subtypes described herein.

The invention therefore provides a compound of the formula (I):

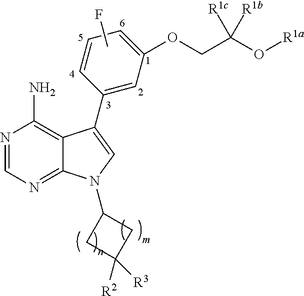

or a pharmaceutically acceptable salt and/or solvate thereof,
wherein
F is fluoro, said fluoro being substituted in the 2, 4 or 6 position of the phenyl ring;
R^{1a} and R^{1b} together with the carbon and oxygen atoms to which they are attached, form a fully saturated ring comprising 2, 3 or 4 further carbon ring atoms, optionally wherein one, several, or all of the hydrogen atoms attached to the carbon ring atoms are replaced with deuterium, and wherein said saturated ring is optionally substituted by 1 or 2 methyl substituents, and R^{1c} is H,
or
R^{1a}, R^{1b} and R^{1c}, together with the atoms to which they are attached, form a fully saturated bridged ring comprising 5 further ring carbon atoms, optionally wherein one, several, or all of the hydrogen atoms attached to the carbon ring atoms are replaced with deuterium;
n and m are both 1, or n and m are both 2;

### R² is H or OH,

R³ is selected from:
—CH₂₍ₚ₎₋heterocyclic², wherein said heterocyclic² is a 4, 5, 6, 7, 8 or 9 membered saturated, unsaturated or partially saturated ring or ring system comprising carbon ring atoms and 1, 2, 3 or 4 ring heteroatoms independently selected from N, O and S, wherein the total number of ring S atoms does not exceed 1, and the total number of ring O atoms does not exceed 1, and said heterocyclic² is optionally substituted with 1, 2, 3 or 4 substituents independently selected from oxo, —C(═O)—C₁-C₄alkyl, —C(═O)—O—C₁-C₄alkyl, C₁-C₄alkyl, fluoro, —C(═O)—NR⁴R⁵ and hydroxy,
   wherein said —C(═O)—C₁-C₄alkyl and C₁-C₄alkyl are each optionally substituted by 1 or 2 hydroxy substituents and/or 1, 2 or 3 fluoro substituents,
—CH₂₍ₚ₎—N(R⁴)—C(═O)—C₁-C₄alkyl,
—CH₂₍ₚ₎—N(R⁴)—C(═O)—O—C₁-C₄alkyl,
—CH₂₍ₚ₎—N(R⁴)—C(═O)—O—C₁-C₆cycloalkyl,
—CH₂₍ₚ₎—N(R⁴)—C₁-C₆cycloalkyl,
—CH₂—OH,
—CH₂₍ₚ₎—NR⁴R⁵
—CH₂₍ₚ₎—N(R⁴)—C(═O)—NR⁴—C₁-C₄alkyl,
   wherein said C₁-C₄alkyl and C₁-C₆cycloalkyl of —CH₂₍ₚ₎—N(R⁴)—C(═O)—C₁-C₄alkyl, —CH₂₍ₚ₎—N(R⁴)—C(═O)—O—C₁-C₆cycloalkyl, and —CH₂₍ₚ₎—N(R⁴)—C₁-C₆cycloalkyl are each optionally substituted by 1, 2, 3 or 4 substituents independently selected from fluoro, methyl and trifluoromethyl,
   or R² and R³, together with the carbon to which they are attached, form a 5 membered saturated heterocyclic ring containing carbon ring atoms and either:
1 or 2 ring N atoms,
1 ring N atom and a ring O atom, or
2 ring O atoms,
   wherein said heterocyclic ring is substituted at a ring carbon atom by an oxo substituent;
   p is 0 or 1;
   R⁴ and R⁵ are each independently selected from H and C₁-C₄alkyl, wherein said C₁-C₄alkyl is optionally substituted with 1, 2 or 3 substituents independently selected from halo and hydroxy.

Unless specified otherwise, the term “compounds of the present invention” refers to compounds of Formula (I) and subformulae thereof, prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

Various embodiments of the invention are described herein. It will be recognized that features specified in each embodiment may be combined with other specified features to provide further embodiments.

In one embodiment of the invention there is provided a compound of formula (I) or a pharmaceutically acceptable salt and/or solvate thereof, wherein

R^{1a} and R^{1b} together with the carbon and oxygen atoms to which they are attached, form a fully saturated ring comprising 2, 3 or 4 further carbon ring atoms, optionally wherein one, several, or all of the hydrogen atoms attached to the carbon ring atoms are replaced with deuterium, and R^{1c} is H,
or
R^{1b} and R^{1c}, together with the atoms to which they are attached, form a fully saturated bridged ring comprising 5 further ring carbon atoms, optionally wherein one, several, or all of the hydrogen atoms attached to the carbon ring atoms are replaced with deuterium;
R³ is selected from:
—CH₂₍ₚ₎₋heterocyclic², wherein said heterocyclic² is a 6, 7 or 8 membered saturated ring or ring system comprising carbon ring atoms and 1, 2 or 3 ring heteroatoms independently selected from N, O and S, wherein the total number of ring S atoms does not exceed 1, and the total number of ring O atoms does not exceed 1, and said heterocyclic² is optionally substituted with 1, 2, 3 or 4 substituents independently selected from oxo, —C(═O)—C₁-C₄alkyl, —C(═O)—O—C₁-C₄alkyl, C₁-C₄alkyl, wherein said —C(═O)—C₁-C₄alkyl is optionally substituted by 1 or 2 hydroxy substituents,
—CH₂₍ₚ₎—N(R⁴)—C(═O)—C₁-C₄alkyl,
—CH₂—OH,
   or R² and R³, together with the carbon to which they are attached, form a 5 membered saturated heterocyclic ring containing carbon ring atoms and either:
1 or 2 ring N atoms,
1 ring N atom and a ring O atom, or
2 ring O atoms,
   wherein said heterocyclic ring is substituted at a ring carbon atom by an oxo substituent;
   p is 0 or 1;
   and
   R⁴ is selected from H and C₁-C₄alkyl.

In another embodiment, there is provided a compound of formula (I), or a pharmaceutically acceptable salt and/or solvate thereof, wherein F is fluoro, said fluoro being substituted in the 2 or 4 position of the phenyl ring.

In another embodiment, there is provided a compound of formula (I), or a pharmaceutically acceptable salt and/or solvate thereof, wherein R^{1a} and R^{1b} together with the carbon and oxygen atoms to which they are attached, form a fully saturated ring comprising 3 or 4 further carbon ring atoms, optionally wherein one, several, or all of the hydrogen atoms attached to the carbon ring atoms are replaced with deuterium, and wherein said saturated ring is optionally substituted by 1 or 2 methyl substituents, and R^{1c} is H.

In another embodiment, there is provided a compound of formula (I), or a pharmaceutically acceptable salt and/or solvate thereof, wherein R^{1a} and R^{1b} together with the carbon and oxygen atoms to which they are attached, form a fully saturated ring comprising 3 or 4 further carbon ring atoms.

In another embodiment, there is provided a compound of formula (I), or a pharmaceutically acceptable salt and/or solvate thereof, wherein R^{1a} and R^{1b} together with the carbon and oxygen atoms to which they are attached, form tetrahydrofuranyl, and R^{1c} is H.

In another embodiment, there is provided a compound of formula (I) or a pharmaceutically acceptable salt and/or solvate thereof, wherein R^{1a}, R^{1b} and R^{1c}, together with the atoms to which they are attached, form a fully saturated bridged ring comprising 5 further ring carbon atoms, optionally wherein one, several, or all of the hydrogen atoms attached to the carbon ring atoms are replaced with deuterium.

In another embodiment, there is provided a compound of formula (I) or a pharmaceutically acceptable salt and/or solvate thereof, wherein R^{1a}, R^{1b} and R^{1c}, together with the atoms to which they are attached, form an optionally deuterated 7-oxa-bicyclo[2.2.1]heptan-1-yl ring system.

In another embodiment, there is provided a compound of formula (I) or a pharmaceutically acceptable salt and/or solvate thereof, wherein R^{1a}, R^{1b} and R^{1c}, together with the atoms to which they are attached, form a fully deuterated 7-oxa-bicyclo[2.2.1]heptan-1-yl ring system.

In another embodiment, there is provided a compound of formula (I), or a pharmaceutically acceptable salt and/or solvate thereof, wherein n and m are both 1.

In another embodiment, there is provided a compound of formula (I), or a pharmaceutically acceptable salt and/or solvate thereof, wherein R² is H.

In another embodiment, there is provided a compound of formula (I), or a pharmaceutically acceptable salt and/or solvate thereof, wherein R³ is —CH₂₍ₚ₎₋heterocyclic², wherein said heterocyclic² is a 6, 7 or 8 membered saturated ring or ring system comprising carbon ring atoms and 1, 2 or 3 ring heteroatoms independently selected from N, O and S, wherein the total number of ring S atoms does not exceed 1, and the total number of ring O atoms does not exceed 1, and said heterocyclic² is optionally substituted with 1, 2, 3 or 4 substituents independently selected from oxo, —C(═O)—C₁-C₄alkyl, —C(═O)—O—C₁-C₄alkyl, C₁-C₄alkyl, wherein said —C(═O)—C₁-C₄alkyl is optionally substituted by 1 or 2 hydroxy substituents.

In another embodiment, there is provided a compound of formula (I), or a pharmaceutically acceptable salt and/or solvate thereof, wherein R³ is
—CH₂₍ₚ₎₋heterocyclic², wherein said heterocyclic² is thiomorpholinyl, piperazinyl or thia-aza-bicyclo[2.2.1]hepantyl, each thiomorpholinyl, piperazinyl or thia-aza-bicyclo[2.2.1]heptanyl being optionally substituted with 1 or 2 substituents independently selected from oxo, —C(═O)—C₁-C₂alkyl, and —C(═O)—O—C₁-C₂alkyl, wherein said —C(═O)—C₁-C₂alkyl is optionally substituted with one hydroxy,
hydroxymethyl-, or
—CH₂₍ₚ₎—NH—C(═O)—C₁-C₂alkyl.

In another embodiment, there is provided a compound of formula (I), or a pharmaceutically acceptable salt and/or solvate thereof, wherein R³ is —CH₂₍ₚ₎₋heterocyclic², wherein said heterocyclic² is thiomorpholinyl, piperazinyl or thia-aza-bicyclo[2.2.1]hepantyl, each thiomorpholinyl, piperazinyl or thia-aza-bicyclo[2.2.1]heptanyl being optionally substituted with 1 or 2 substituents independently selected from oxo, —C(═O)—C₁-C₂alkyl, and —C(═O)—O—C₁-C₂alkyl, wherein said —C(═O)—C₁-C₂alkyl is optionally substituted with one hydroxy.

In a particular embodiment there is provided a compound of formula (I) wherein

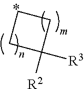

forms

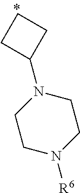

wherein R⁶ is optionally substituted with 1 substituent selected from oxo, —C(═O)—C₁-C₄alkyl, —C(═O)—O—C₁-C₄alkyl, C₁-C₄alkyl, fluoro, —C(═O)—NR⁴R⁵ and hydroxy, wherein said —C(═O)—C₁-C₄alkyl and C₁-C₄alkyl are each optionally substituted by 1 or 2 hydroxy substituents and/or 1, 2 or 3 fluoro substituents.

In a further embodiment, there is provided a compound of formula (I), or a pharmaceutically acceptable salt and/or solvate thereof, wherein R³ is —CH₂₍ₚ₎₋heterocyclic², wherein said heterocyclic² is piperazinyl, optionally substituted by C(═O)—CH₂—OH or C(═O)—CH(OH)—CH₃.

In another embodiment, there is provided a compound of formula (I), or a pharmaceutically acceptable salt and/or solvate thereof, wherein R³ is selected from hydroxymethyl- and —CH₂₍ₚ₎—NH—C(═O)—C₁-C₂alkyl.

In another embodiment, there is provided a compound of formula (I), or a pharmaceutically acceptable salt and/or solvate thereof, wherein R² and R³, together with the carbon to which they are attached, form a 5 membered saturated heterocyclic ring containing carbon ring atoms and either:
1 or 2 ring N atoms,
1 ring N atom and a ring O atom, or
2 ring O atoms,
   wherein said heterocyclic ring is substituted at a ring carbon atom by an oxo substituent.

In another embodiment, there is provided a compound of formula (I), or a pharmaceutically acceptable salt and/or solvate thereof, wherein R² and R³, together with the carbon to which they are attached, form

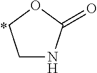

wherein * indicates the point of attachment.

In another embodiment, there is provided a compound of formula (I), or a pharmaceutically acceptable salt and/or solvate thereof, wherein R⁴ is H.

In another embodiment individual compounds according to the invention are those listed in the Examples section below.

“a salt and/or solvate thereof” includes a salt thereof, a solvate thereof, and a solvate of the salt thereof.

In another embodiment, there is provided a compound of formula (I) according to any one of the embodiments herein, or a salt thereof.

“optionally substituted by 1 or 2 hydroxy substituents and/or 1, 2 or 3 fluoro substituents” includes substitution by both hydroxy and fluoro substituents, substitution by only hydroxy substituents and substitution by only fluoro substituents.

F is fluoro.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabelled forms as well as isotopically labeled forms of the compounds. Isotopically labelled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ⁶⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective at (1) at least partially alleviating, inhibiting, preventing and/or ameliorating a condition, or a disorder or a disease (i) mediated by IGF-1R, or (ii) associated with IGF-1R activity, or (iii) characterized by activity (normal or abnormal) of IGF-1R; or (2) reducing or inhibiting the activity of IGF-1R; or (3) reducing or inhibiting the expression of IGF-1R.

In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of IGF-1R; or at least partially reducing or inhibiting the expression of IGF-1R.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder. In the case of cancer, the term “treat”, “treating” or “treatment” may mean the reduction of the number of cancer cells; reduction of the tumor size; inhibition of (i.e., slow to some extent and preferably stop) cancer cell infiltration into peripheral organs including soft tissue and bone; inhibit ion of (i.e., slow to some extent and preferably stop) tumor metastasis; inhibit, to some extent, tumor growth; reduced morbidity and mortality; improvement in quality of life issues; and/or to relieve to some extent one or more of the symptoms associated with the cancer.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70 enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

Typically, the compounds of formula (I) can be prepared according to the Schemes provided herein.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

In another aspect, the present invention provides a pharmaceutical composition comprising a compound of formula (I), or a pharmaceutically acceptable salt and/or solvate thereof, and one or more pharmaceutically acceptable carriers.

In another aspect, the present invention provides a pharmaceutical composition comprising a therapeutically effective amount of a compound of formula (I), or a pharmaceutically acceptable salt and/or solvate thereof, and one or more pharmaceutically acceptable carriers.

In another aspect, the present invention provides a pharmaceutical composition for treating a disease or disorder mediated by IGF-1R comprising a compound of formula (I), or a pharmaceutically acceptable salt and/or solvate thereof, as an active ingredient.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be desirable.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (a g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of formula I in free form or in salt form, exhibit valuable pharmacological properties, e.g. IGF-1R modulating properties, e.g. as indicated in tests as provided in the next sections and are therefore indicated for therapy.

Compounds of the invention may be useful in the treatment of an indication selected from cancer, such as carcinoma, lymphoma, blastoma, and leukemia; more particular examples of cancers including, but are not limited to: chronic lymphocytic leukemia (CLL), acute myeloid leukemia (AML), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), adrenocotical carcinoma (ACC), medulloblastoma and desmoid; multiple myeloma, neuroblastoma, synovial, hepatocellular, Ewing's Sarcoma, adrenocotical carcinoma (ACC), or a solid tumor selected from osteosarcoma, melanoma, tumor of breast, renal, prostate, colorectal, thyroid, ovarian, pancreatic, lung, uterine or gastrointestinal tumor, and or non-cancer indications such as acute lung injury and pulmonary fibrosis and diabetic retinopathy, all of which are examples of indications that may be mediated by IGF-1R.

To the extent the drug may prevent growth and/or kill existing cancer cells, it may be cytostatic and/or cytotoxic.

The term “cancer” refers to the physiological condition in mammals that is typically characterized by unregulated cell growth/proliferation.

Thus, as a further embodiment, the present invention provides:
a compound of the formula (I) as defined herein, as a medicament/for use as medicament;
a compound of the formula (I) as defined herein, for the treatment of/for use in the treatment of one or more IGF-1R mediated disorders or diseases;
the use of a compound of formula (I) as defined herein, for the manufacture of a medicament for the treatment of an IGF-1R mediated disorder or disease;
the use of a compound of formula (I) as defined herein, for the treatment of an IGF-1R mediated disorder or disease;
the use of a compound of formula I as defined herein for the inhibition of the IGF-IR tyrosine kinase;
the use of a compound of formula (I) as defined herein, for the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of a disorder or disease selected from acute lung injury, pulmonary fibrosis and diabetic retinopathy;
a method of modulating IGF-1R activity in a subject, comprising the step of administering to a subject a therapeutically effective amount of a compound of formula I as defined herein;
a method for the treatment of an IGF-1R mediated disorder or disease comprising the step of administering to a subject a therapeutically effective amount of a compound of formula (I) as defined herein;
a method for inhibition IGF-1R in a cell, comprising contacting said cell with an effective amount of a compound of formula I as defined herein.
A method, use, or compound for use, as defined above, wherein the IGF-1R mediated disorder or disease is cancer.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a combination comprising a compound of formula (I), or a pharmaceutically acceptable salt and/or solvate thereof, and one or more therapeutically active co-agents. Optionally, the pharmaceutical composition may comprise a pharmaceutically acceptable excipient, as described above.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by IGF-1R. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula (I) for treating a disease or condition mediated by IGF-1R, wherein the medicament is prepared for administration with another therapeutic agent. The invention also provides the use of another therapeutic agent for treating a disease or condition mediated by IGF-1R, wherein the medicament is administered with a compound of formula (I).

The invention also provides a compound of formula (I) for use in a method of treating a disease or condition mediated by IGF-1R, wherein the compound of formula (I) is prepared for administration with another therapeutic agent. The invention also provides another therapeutic agent for use in a method of treating a disease or condition mediated by IGF-1R, wherein the other therapeutic agent is prepared for administration with a compound of formula (I). The invention also provides a compound of formula (I) for use in a method of treating a disease or condition mediated by IGF-1R, wherein the compound of formula (I) is administered with another therapeutic agent. The invention also provides another therapeutic agent for use in a method of treating a disease or condition mediated by IGF-1R, wherein the other therapeutic agent is administered with a compound of formula (I).

The invention also provides the use of a compound of formula (I) for treating a disease or condition mediated by IGF-1R, wherein the patient has previously (e.g. within 24 hours) been treated with another therapeutic agent. The invention also provides the use of another therapeutic agent for treating a disease or condition mediated by IGF-1R, wherein the patient has previously (e.g. within 24 hours) been treated with a compound of formula (I).

In one embodiment, the other therapeutic agent is an antiproliferative agent.

The term “antiproliferative agent” includes, but are not limited to, aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, lipid kinase inhibitors or compounds decreasing lipid kinase activity, eg PI3 kinase inhibitors, antineoplastic antimetabolites, platin compounds, compounds decreasing the protein kinase activity, eg mTOR inhibitors, Raf inhibitors, MEK inhibitors, and further anti-angiogenic compounds, gonadorelin agonists, anti-androgens, bengamides, bisphosphonates and trastuzumab, and radiotherapy.

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEX™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

A combination of the invention comprising an antineoplastic agent which is an aromatase inhibitor is particularly useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™. Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™ Mitoxantrone can be administered, e.g., in the form as it is marketed, e.g. under the trademark NOVANTRON™.

The term “lipid kinase inhibitor” or “a compound decreasing lipid kinase activity” relates to PI3 kinase inhibitors, PI4 kinase inhibitors, Vps34 inhibitors. Specific examples include: NVP-BEZ235, NVP-BGT226, NVP-BKM120, AS-604850, AS-041164, AS-252424, AS-605240, GDC0941, PI-103, TGX221, YM201636, ZSTK474, examples described in WO 2009/080705 and US 2009/163469.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®) and rofecoxib (Vioxx®).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCl-779 and ABT578.

The term “antineoplastic antimetabolites” includes, but is not limited to 5-fluorouracil, 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity” and “further anti-angiogenic compounds” as used herein includes, but is not limited to compounds which decrease the activity of e.g. the Vascular Endothelial Growth Factor (VEGF), the Epidermal Growth Factor (EGF), and c-Src and anti-angiogenic compounds having another mechanism of action than decreasing the protein kinase activity. Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula I), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl.

Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin, described by M. S. O'Reilly et al, Cell 79, 1994, 315-328; and Endostatin, described by M. S. O'Reilly et al, Cell 88, 1997, 277-285; sorefanib (Nexavar), Sutent (sunitinib), BAY 43-9006. Compounds which decrease the activity of EGF are especially compounds which inhibit the EGF receptors, especially the tyrosine kinase activity of the EGF receptors, and compounds binding to EGF, and are in particular those compounds generically and specifically disclosed in WO 97/02266 (describing compounds of formula IV), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980. Specific EGF receptor inhibitor examples include, but not limited to; Tarceva (erlotinib), Iressa (Gefitinib), Tywerb (lapatanib). Erbitux (cetuximab), Avastin (bevacizumab), Herceptin (trastuzamab), Rituxan (rituximab), Bexxar (tositumomab), panitumumab. Compounds which decrease the activity of c-Src include, but are not limited to, compounds inhibiting the c-Src protein tyrosine kinase activity as defined below and to SH2 interaction inhibitors such as those disclosed in WO97/07131 and WO97/08193; compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;

Compounds which decrease the activity of Raf kinases include, but are not limited to: Raf265, sorefanib, BAY 43-9006.

Compounds which inhibit downstream effectors of Raf kinases, such as MEK. Examples of MEK inhibitors include; PD 98059, AZD6244 (ARRY-886), CI-1040, PD 0325901, u0126.

Anti-angiogenic compounds having another mechanism of action than decreasing the protein kinase activity include, but are not limited to e.g. thalidomide (THALOMID™), SU5416, and celecoxib (Celebrex™)

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™ Abarelix can be formulated, eg. as disclosed in U.S. Pat. No. 5,843,901.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having aniproliferative properties and includes, but is not limited to the compounds generically and specifically disclosed in WO00/29382, preferably, to the compound disclosed in Example 1 of WO00/29382.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™ “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETA™.

“Trastuzumab” can be administered, e.g., in the form as it is marketed, e.g. under the trademark HERCEPTIN™.

For the treatment of AML, compounds of formula I can be used in combination with standard leukemia therapies, especially in combination with therapies used for the treatment of AML. In particular, compounds of formula I can be administered in combination with e.g. farnesyltransferase inhibitors and/or other drugs used for the treatment of AML, such as Daunorubicin, Adriamycin, Ara-C, VP-16, Teniposide, Mitoxantrone, Idarubicin and Carboplatinum.

In one further embodiment, the additional active ingredient is a hormonal medicine.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of formula I, can be prepared and administered as described in the art such as in the documents cited above.

The activity of a compound according to the present invention can be assessed by the following in vitro & in vivo methods.

IVa. Cellular IGF-1R and InsR Assays

Compound-mediated inhibition of IGF1R and INSR phosphorylation in Hek293 cells transduced with the corresponding receptors is assessed in a capture ELISA format using the MSD (Meso Scale Discovery) platform. Briefly, 30′000 cells washed and diluted in starvation medium (DMEM high glucose supplemented with 0.1% BSA) are seeded in 90 μL per well into 96-well plates pre-coated with poly-D-lysine (0.1 mg/mL in PBS/O). After 24 h incubation at 37° C. and 5% CO2, dose-response effects are determined with 3-fold serial compound dilutions, starting at 10 μM. The final vehicle concentration is 0.1% DMSO in all wells. Following pre-incubation with compounds for 1 h, receptor phosphorylation is triggered by a 10 min exposure to 1.0 ng/μL IGF for Hek293-IGF1R cells, and 5.0 ng/μL insulin for Hek293-InsR cells. Cell lysis is achieved by addition of 80 μL MSD lysis buffer per aspirated well, incubation on ice for 20 min, and a freeze-thaw cycle. Target phosphorylation is then assessed by transferring volumes corresponding to approx. 6 μg Hek293-IGF1R or 0.6 μg Hek293-InsR lysates to MSD assay plates pre-coated with total-IGF1R or total-InsR Abs, respectively. After incubation for 2 h at rt, wells are exposed for 1 hr to a rabbit monoclonal antibody (CST #3024, 1:1000) detecting pIGF1R(Tyr1135/1136) as well as pINSR(Tyr1150/1151). Immune complexes are detected by a SULFO-Tag™-coupled anti-rabbit IgG antibody in the presence of 150 μL MSD read-buffer. Light emission at 620 nm triggered by application of electric current is recorded on a MSD Sectorlmager 6000. Acquired raw data (mean Ru-ECL units) are processed in an Excel analysis template. The plate blank (MSD lysis buffer) is subtracted from all data points. The effect of a particular test compound concentration on receptor phosphorylation is expressed relative to the window defined by ligand-stimulated vs unstimulated control cells (set as 100%). IC50 values [nM] are determined using 4-parametric curve-fitting (XLfit software, V4.3.2).

Test results obtained using the above describe method are summarized in the table herein.

### IVb Enzymatic Assays

Two methods were used to analyze phosphorylated peptides and proteins produced by the listed tyrosine and serine/threonine-specific protein kinases: either using a filter-binding assay (FB), or using a flashplate assay (FP). The activities of protein kinases were assayed in the presence or absence of inhibitors, by measuring the incorporation of ³³P from [γ³³P]ATP into appropriate substrates.

### Filter-Binding Assay

96-well polypropylene microplates were used to assay the activity in the FB mode. To determine the inhibitory activity of compounds 10 μL of compound dilutions were pipetted into 96-well plates followed by the addition of 10 μL of assay mix and 10 μL of individual enzymes. With the addition of the enzymes the reactions were initiated and continued at RT. The reactions were stopped by the addition of 50 μL of a 125 mM EDTA solution pH 8.0. The final concentration of DMSO in the enzyme assays was 1%.

### Flashplate Assay

Flashplates are available as 96-well standard (STFPs) or as streptavidin-(SAFPs) or nickel coated FPs (NiFPs) from Perkin Elmer. STFPs are 96-well polystyrene microplates in which the interior of each well is permanently coated with a thin layer of polystyrene-based scintillant. Streptavidin flashplates (SAFP) are 96 or 384-well STFPs coated with streptavidin. SAFPs are suitable for a wide variety of assay applications which utilize biotinylated capture molecules. NiFP or nickel chelate flashplates are 96- or 384-well STFPs coated with nickel chelate. NiFPs are designed for in-plate, radiometric assays which utilize 4- or 6-histidine tagged proteins and peptides.

All kinase assays were performed in STFPs for 60 mins at RT and stopped with 50 μL of 0.5% H₃PO₄ except PKA which were carried out in polypropylene 96- and 384-well plates, respectively. PKA assays were stopped with 50 μL of 125 mM EDTA (pH 8.0) and 50 μL were transferred to either SAFPs or NIFPs to capture the biotinylated or histidine tagged peptides phosphorylated by PKA (SAFP) or by NiFPs. All wells were then washed three times with 200 μL of 0.5% H₃PO₄ and the plates were dried at room temperature. The plates were sealed and counted in a microplate scintillation counter (TopCount NXT, TopCount NXT HTS). The final concentration of DMSO in the enzyme assays was 1%.

### Format and Incubation (Min) of the Protein Kinase Assays

| | Filter-binding, | Flashplate, | Flashplate, |
|---|---|---|---|
| Enzyme | 96 wells | 96 wells | 384 wells |
| c-Abl-T315I | 10 | 30 | 30 |
| CDK2/A (Bt-P) | 30 | 30 | NT |
| c-Kit | 10 | 30 | 30 |
| c-Met | 15 | 30 | 30 |
| EphA4 | 10 | NT | NT |
| FGFR-4 | 10 | NT | NT |
| HER-2 (Erb B2) | 15 | 30 | 30 |
| IGF-1R | 20 | 30 | 30 |
| Ins-R | 20 | 30 | 30 |
| JAK-2 | 10 | 30 | 30 |
| KDR | 10 | 30 | 30 |
| P38 | 10 | NT | NT |
| PKA (Bt-P) | 45 | 60 (SAFP) | 60 (SAFP) |
| Ret | 10 | 30 | 30 |

NT: not tested

hERB Binding Assay

Test compounds compete for [³H]dofetilide binding to a crude membrane preparation of HEK293 cell membranes stably transfected with hERG channels. The assay runs in a 96-well filtration format. [³H]dofetilide binds to hERG membranes with an apparent K_{d} of 10±4.7 nM and a Bₘₐₓ of 4.7±1.6 pmol/mg protein. A good correlation (r²=0.72, n=40) was achieved when comparing the binding data of ‘gold standard’ drugs with published patch clamp data.

The following are pipetted into each well of a 96-well Millipore GF/C filter plate: 119 μl assay buffer, 1 μl test compound in 100% DMSO (or 100% DMSO only for total binding), 40 μl [3H]dofetilide (12.5 nM, final concentration 2.5 nM); 40 μl crude membrane suspension (ca. 15 μg protein). The final concentration of DMSO during the incubation is 0.5%. Incubations are performed at room temperature (21-23° C.) for 90 min. Non-specific binding (NSB) is defined as the binding remaining in the presence of 25 μM terfenadine. The incubations are terminated by rapid filtration on a Millipore filtration manifold, followed by three washes of 200 μl ice-cold assay buffer. The rubber base plate of the filter plate is removed and the plate is left at 40° C. for at least 1 hr for the filters to dry. Then a clear base plate is attached to the bottom (Multiscreen liner, Wallac 1450-433), 40 μl scintillant (MicroScint-20) is added and the plate is sealed (Sealing Tape SI, Nunc 236366). The plates are then read in a Wallac MicroBeta Trilux beta-counter for 1.5 min per well. Compounds are tested as ten-concentration response curves, ranging from 30 μM to 1 nM in 1:3 and 1:3.333 dilution steps. Dilution curves are prepared in 2.5% DMSO, 5× the desired final concentration. The reference compound (terfenadine) is tested as an eight concentrationresponse curve, ranging from 10 μM to 0.6 nM in 1:4 dilution steps.

Preferred compounds of the invention have a favourable profile in the hERB binding assay. In particular, compounds of the present invention have a more favourable hERB binding profile than compounds of the prior art.

Test results obtained using the above describe methods are summarized in the tables below.

### Enzymatic Kinase Selectivities

| Kinase/ | IC₅₀ values (nM) | | | |
|---|---|---|---|---|
| assay | Example 1 | Example 6 | Example 10 | Example 18 |
| IGF1R | 4.2 | 3.8 | 13 | 15 |
| InsR | 38 | 39 | 81 | 43 |
| Aurora A | 7 000 | >10 000 | >10 000 | >10 000 |
| cABLT315 | >10 000 | >10 000 | >10 000 | >10 000 |
| CDK2A | >10 000 | >10 000 | >10 000 | >10 000 |
| cKIT | >10 000 | 770 | >10 000 | >10 000 |
| cMET | >10 000 | >10 000 | >10 000 | >10 000 |
| COT1 | >10 000 | >10 000 | >10 000 | >10 000 |
| EphA4 | >10 000 | >10 000 | >10 000 | >10 000 |
| ERK2 | >10 000 | >10 000 | >10 000 | >10 000 |
| FGFR-4 | >10 000 | >10 000 | >10 000 | >10 000 |
| GSK3beta | >10 000 | >10 000 | >10 000 | >10 000 |
| HER2 | >10 000 | >10 000 | >10 000 | >10 000 |
| JAK2 | >10 000 | >10 000 | >10 000 | >10 000 |
| KDR | 2 400 | 5 700 | >10 000 | >10 000 |
| LCK | >10 000 | 8 600 | 6 600 | >10 000 |
| MK2 | >10 000 | >10 000 | >10 000 | >10 000 |
| P38a | >10 000 | >10 000 | >10 000 | >10 000 |
| PDGFRa | 6 300 | 8 300 | >10 000 | >10 000 |
| PKA | >10 000 | >10 000 | >10 000 | >10 000 |
| RET | 2 000 | 3 400 | 6 400 | >10 000 |
| TYK2 | >10 000 | >10 000 | >10 000 | >10 000 |
| hERG binding | — | >30 000 | 6 800 | >30 000 |

| Kinase/ | IC₅₀ values (nM) | | | |
|---|---|---|---|---|
| assay | Example 40 | Example 34 | Example 36 | Example 27 |
| IGF1R | 8.7 | 8.4 | 30 | 22 |
| InsR | 270 | 75 | 1300 | 130 |
| Aurora A | >10 000 | >10 000 | >10 000 | >10 000 |
| cABLT315 | >10 000 | >10 000 | >10 000 | >10 000 |
| CDK2A | >10 000 | >10 000 | >10 000 | >10 000 |
| cKIT | >10 000 | >10 000 | >10 000 | >10 000 |
| cMET | >10 000 | >10 000 | >10 000 | >10 000 |
| COT1 | >10 000 | >10 000 | >10 000 | >10 000 |
| EphA4 | >10 000 | >10 000 | >10 000 | >10 000 |
| ERK2 | >10 000 | >10 000 | >10 000 | >10 000 |
| FGFR-4 | >10 000 | >10 000 | >10 000 | >10 000 |
| GSK3beta | >10 000 | >10 000 | >10 000 | >10 000 |
| HER2 | >10 000 | >10 000 | >10 000 | >10 000 |
| JAK2 | >10 000 | >10 000 | >10 000 | >10 000 |
| KDR | 9 200 | >10 000 | >10 000 | >10 000 |
| LCK | >10 000 | >10 000 | 9 700 | 6 600 |
| MK2 | >10 000 | >10 000 | >10 000 | >10 000 |
| P38a | >10 000 | >10 000 | >10 000 | >10 000 |
| PDGFRa | >10 000 | >10 000 | >10 000 | >10 000 |
| PKA | >10 000 | >10 000 | >10 000 | >10 000 |
| RET | >10 000 | >10 000 | 3 100 | >10 000 |
| TYK2 | >10 000 | >10 000 | >10 000 | >10 000 |
| hERG binding | >30 000 | — | >30 000 | >30 000 |

| | IC₅₀ values (nM) | | | |
|---|---|---|---|---|
| | | | | Example 1 |
| Kinase/ | | | | WO |
| assay | Example 4 | Linsitinib | BMS 754807 | 2008/117051 |
| IGF1R | 13 | 14 | 29 | 22 |
| InsR | 250 | 31 | 16 | 21 |
| Aurora A | >10 000 | 6 950 | <3 | 16 |
| cABLT315 | >10 000 | >10 000 | 47 | 260 |
| CDK2A | >10 000 | >10 000 | >10 000 | >10 000 |
| cKIT | >10 000 | >10 000 | >10 000 | >10 000 |
| cMET | >10 000 | >10 000 | 380 | 70 |
| COT1 | >10 000 | >10 000 | >10 000 | >10 000 |
| EphA4 | >10 000 | >10 000 | 250 | 420 |
| ERK2 | >10 000 | >10 000 | >10 000 | >10 000 |
| FGFR-4 | >10 000 | >10 000 | 1 300 | >10 000 |
| GSK3beta | >10 000 | >10 000 | 82 | 3 600 |
| HER2 | >10 000 | >10 000 | >10 000 | >10 000 |
| JAK2 | >10 000 | >10 000 | 5 200 | 900 |
| KDR | 4 500 | 1 400 | 580 | 4 000 |
| LCK | >10 000 | 3 900 | 430 | 5 300 |
| MK2 | >10 000 | >10 000 | >10 000 | >10 000 |
| P38a | >10 000 | >10 000 | >10 000 | >10 000 |
| PDGFRa | >10 000 | >10 000 | >10 000 | >10 000 |
| PKA | >10 000 | >10 000 | 6 400 | 2 100 |
| RET | >10 000 | 5 400 | 170 | 1 100 |
| TYK2 | >10 000 | >10 000 | 5 200 | 700 |
| hERG binding | 8 700 | 2 650 | >30 000 | >30 000 |

### Metabolic Stability

The compounds of the invention show improved efficacy and tolerability when compared to known IGF-1R inhibitors. Metabolic factors are anticipated to contribute to the observed improvements in efficacy and tolerability.

Effects of Metabolism: Known compounds have been shown to produce desirable effects in in-vivo models through the inhibition of IGF-1 receptor activity, but they have been found to undergo extensive metabolism at the methylene group of the benzyl ether leading to cleavage to the corresponding phenol metabolite X:

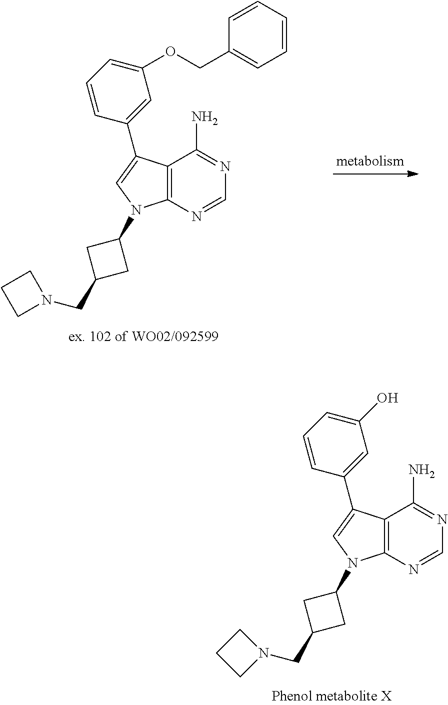 [SMILES: Nc1ncnc2c1c(-c1cccc(OCc3ccccc3)c1)cn2[C@H]1C[C@@H](CN2CCC2)C1 ; Nc1ncnc2c1c(-c1cccc(O)c1)cn2[C@H]1C[C@@H](CN2CCC2)C1]

This not only limits the pharmacokinetic profile of such derivatives, but also generates phenolic metabolites, such as the specific example X, which shows multiple potent kinase activities, as shown in the following test.

IC₅₀ (nM)

| Kinase | WO02/092599, ex. 102 | Phenol metabolite X |
|---|---|---|
| IGF-1R | 82 | 420 |
| InsR | 230 | 3800 |
| C-Abl | >10 000 | 47 |
| c-src | >10 000 | 4 |
| EphB4 | >10 000 | 9 |
| RET | 4400 | 8 |

A further preferred embodiment of the invention provides a compound of formula (I) which shows increased metabolic stability, and/or from which the formation of the phenol metabolite is reduced or avoided.

### Met ID Iv Vitro Incubation Method

The metabolism of unlabelled compound was examined following in vitro incubations with liver microsomes from rat and human. The samples were analyzed by capillary-HPLC/MS⁽ⁿ⁾ and screened for potential metabolites of the compound.

In vitro

| | |
|---|---|
| Microsomal | Protein: 0.3 mg/ml; Alamethicin: 1.25 μM; UDPGA: |
| incubation | 2.4 mM GSH: 0, 1.5, 10 mM; Parent: 5 μM, NADPH- |
| | Reg. system |
| Species: | Rat, human |
| Incubation | 1 h |
| time | |
| Analytical | Capillary-HPLC/MS(-MS) |
| method | |
| MS | MS: LTQ XL (MS/MS) |
| | ITFS: 200-1500 amu |
| | ITMS2-5: 20-35% rel. coll. energy and wideband |
| | activation, |
| | Isolation width: 1.5 amu |
| HPLC | Column switching: Reprosil ODS3 C18, 5 μM, 0.4 × |
| column/temp. | 11 mm |
| | Column: Reprosil ODS3 C18, 3 μM, 0.3 × 150 mm |
| | Analytical column thermostated at +40° C. |
| Mobile phase | A (H₂O/ACN, 95/5 + 0.1% CH₃COO NH₄⁺ 0.02% TFA) |
| | B (H₂O/ACN/MeOH, 5/47.5/47.5 + 0.1% CH₃COO |
| | NH₄⁺ 0.02% TFA) |
| | D₂O experiment performed, H/D exchange confirmed for |
| | all metabolites |
| Gradient | 5-95% B, 2-30 min at 4.5 μL/min (Pump: Chorus220) |

### Experimental:

The following examples are intended to illustrate the invention and are not to be construed as being limitations thereon. Temperatures are given in degrees centrigrade. Unless otherwise indicated, the reactions take place at room temperature. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### ABBREVIATIONS

DMF N,N-dimethylformamide
DCM dichloromethane
THF tetrahydrofuran
NIS N-iodosuccinimide
DIBAL-H diisobutylaluminium hydride
DIPEA/DIEA diisopropylethylamine
DEA diethylamine
HATU O-(7-azabenzotriazol-1-yl)-1,1,3,3-tetramethyluronium hexafluorophosphate

### EXAMPLES

The following Examples serve to illustrate the invention without limiting its scope. Abbreviations used are those conventional in the art.

### I Analytical Methods

The following HPLC, HPLC/MS and MS methods are used in the preparation of the Intermediates and Examples:

### HPLC Methods:

### Method A

HPLC linear gradient between A=H₂O/TFA 1000:1 and B=acetonitrile/TFA 1000:1 Grad 1: 2-100% Bin 4.5 min and 1 min at 100% B; column: Chromolith Performance 100 mm×4.5 mm (Merck, Darmstadt, Germany); flow rate 2 ml/min. Detection at 215 nM.

### Method B

### Column: Speed ROD RPI8e, 50×4.6 mm.

Flow rate: 1.3 ml/min
Mobile phase: A) TFA/water (0.1/100, v/v), B) TFA/acetonitrile (0.1/100, v/v)
Gradient: linear gradient from 0% B to 100% B in 6 min then 2 min 100% B

### Detection: UV at 215 nm

### MS Methods:

### Method L

### Micromass Platform II

### Range DA 200-900

### Cone+30 V and −30 V

Pump Agilent 1100 Quat, 2 min, 0.05 ml/min 1:1 methanol: 15% methanol in water, containing 0.2% ammonium hydroxide (25%).

### Injector, CTC PAL

### Method M

### Agilent G1379A Degasser

### Agilent G1312A Binary Pump

### Agilent G1367A Well Plate Auto Sampler

### Agilent G1316A Column Heater

### Agilent G1315B Diode Array Detector

### Agilent G1496C MSD

### Sedex 75 Evaporative Light Scattering Detector

### Mobile Phase: H₂O+0.05% TFA and Acetonitrile+0.035% TFA

Gradient: 1 mL/minute, initial 10% ACN to final 90% ACN in 3 minutes, 100% B for 0.49 minutes, 100% B to initial 10% B in 0.1 minute. The column is re-equilibrated in the ˜45 seconds between injections.
MS Scan: 150 to 1000 amu in 1 second
Diode Array Detector: monitors 220 nm, 254 nm, and 280 nm

### Preparative HPLC Methods:

### Method R

Gilson preparative HPLC system, with UV-triggered collection system
Column, Sunfire Prep C18 OBD 5 microm 30×100 mm, temperature 25 ce
Eluent, gradient from 5-100% acetonitrile in 0.05% aqueous trifluoroacetic acid over 20 minutes, flow rate 30 ml/min.

### Detection UV 254 nm

### Method S

### Instrument: Waters 2525 Binary Pump, Waters 515 Make Up Pump, Waters 2767 Auto Sampler/Fraction Collector, Waters 2487 Dual Wavelength UV Detector, Waters ZQ

### Mass Spectrometer

Mass triggered collection system.

### Mobile Phase: H₂O+0.05% TFA (A), Acetonitrile+0.035% TFA (B)

### UV Detector: 220 nm and 254 nm

MS Scan: 180 to 800 amu in 0.5 seconds

### Gradient:

| Time (min) | Flow Rate (mL/min) | % B |
|---|---|---|
| 0 | 20 | 10 |
| 1.4 | 20 | 10 |
| 1.45 | 100 | 10 |
| 3.99 | 100 | 40 |
| 4 | 100 | 100 |
| 4.15 | 100 | 100 |
| 4.16 | 100 | 10 |
| 4.2 | 10 | 10 |
| 4.25 | 10 | 10 |

### HPLC/MS Methods:

### Method X

### ZQ 2000

Range Da 100-900 (positive) and 120-900 (negative) Cone+17 V and 17 V
Pump Agilent 1100 Bin, 3.5 minute run time, channel A water with 5% acetonitrile, channel B acetonitrile, containing 0.5-1.0% formic acid

| Time (min) | Flow Rate (mL/min) | % B |
|---|---|---|
| 0 | 1.2 | 10 |
| 1.4 | 1.4 | 95 |
| 1.45 | 2.4 | 95 |
| 3.99 | 2.4 | 10 |

Injector, CTC PAL, 5 microl

### Oven Agilent 1100, 50 ce

Column, Waters XBridge, 3×30 mm, 2.5 microm, C18

### Detector, Agilent 1100 DAD, 210-350 nm

Method Y

Instrument: Agilent G1379A Degasser, Agilent G1312A Binary Pump, Agilent G1367A Well Plate Auto Sampler, Agilent G1316A Column Heater, Agilent G1315B Diode Array Detector, Agilent G1496C MSD, Sedex 75 Evaporative Light Scattering Detector

### Eluent:

A: Water+0.05% Formic Acid+0.05% Ammonium acetate (7.5 M solution)

### B: Acetonitrile+0.04% Formic Acid

### Column

### Ascentis Express RP-Amide 2.7 um 2.1×30 mm @ 50° C.

### Gradient

Flow: 1.2 ml/min

| Time | % B |
|---|---|
| 0 | 2 |
| 1.7 | 98 |
| 2.15 | 98 |
| 2.19 | 2 |

UV detection, DAD 210-350 nm
MS detection, 100-900 m/z

### II Chemical Synthesis—Intermediates

### Intermediate A: 2-[2-Fluoro-3-(tetrahydro-furan-2-ylmethoxy)-phenyl]-4,4,5,5-tetramethyl-[1,3,2]dioxaborolane

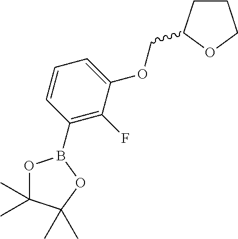 [SMILES: CC1(C)OB(c2cccc(OCC3CCCO3)c2F)OC1(C)C]

A mixture of 2-(3-bromo-2-fluoro-phenoxymethyl)-tetrahydrofuran (Step A.1, 776 mg, 5.59 mmol), bis-(pinacolato)diboron (1.576 g, 6.21 mmol), potassium acetate (0.83 g, 8.46 mmol) and 1,1″-bis(diphenylphosphino)ferrocenedicholoro palladium (II) dichloromethane complex (62 mg, 0.085 mmol) in DMF (31 ml) was purged with argon and then heated for 18 hours at 80° C. under an argon atmosphere. The cooled reaction mixture was then filtered through Hyflo, washing with DMF, and evaporated. Purification of the residue by normal phase chromatography, eluting with a DCM/methanol gradient, to give the title compound as a beige solid.

### Step A.1: 2-(3-Bromo-2-fluoro-phenoxymethyl)-tetrahydrofuran

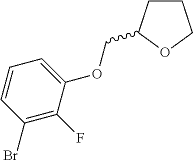 [SMILES: Fc1c(Br)cccc1OCC1CCCO1]

A mixture of 3-bromo-2-fluorophenol (0.78 g, 4.00 mmol), 2-(bromomethyl)tetrahydrofuran (1.7 g, 9.99 mmol), K₂CO₃ (0.71 g, 5.00 mmol), and DMF (10 ml) was heated for 2 hours at 130° C. under an argon atmosphere. The cooled reaction mixture was diluted with ethyl acetate and washed with water then brine, the organic layers dried over Na₂SO₄ and evaporated. Purification of the residue by normal phase column chromatography, eluting with a gradient of ethyl acetate in hexane, gave the title compound as a colourless oil. ¹H-NMR (400 MHz, DMSO-d₆): δppm 7.24-7.17 (m, 2H), 7.11-7.06 (m, 1H), 4.21-4.15 (m, 1H), 4.10-3.99 (m, 2H), 3.83-3.72 (m, 1H), 3.71-3.61 (m, 1H), 2.05-1.95 (m, 1H), 1.92-1.78 (m, 2H), 1.73-1.63 (m, 1H).

### Intermediate B: 2-{2-Fluoro-3-[(R)-1-(tetrahydro-furan-2-yl)methoxy]-phenyl}-4,4,5,5-tetramethyl-[1,3,2]dioxaborolane

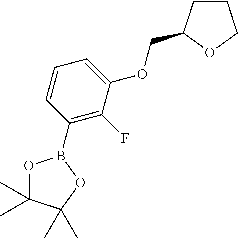 [SMILES: CC1(C)OB(c2cccc(OC[C@H]3CCCO3)c2F)OC1(C)C]

The title compound was prepared in an analagous fashion to Intermediate C by substituting (S)-2-tetrahydrofurylmethanol with (R)-2-tetrahydrofurylmethanol.

### Intermediate C: 2-{2-Fluoro-3-[(S)-1-(tetrahydro-furan-2-yl)methoxy]-phenyl}-4,4,5,5-tetramethyl-[1,3,2]dioxaborolane

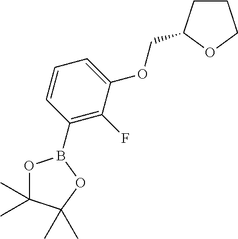 [SMILES: CC1(C)OB(c2cccc(OC[C@@H]3CCCO3)c2F)OC1(C)C]

A mixture of (S)-2-(3-bromo-2-fluoro-phenoxymethyl)-tetrahydrofuran (Step C.1, 3.49 g, 12.69 mmol), bis-(pinacolato)diboron (3.22 g, 12.69 mmol), potassium acetate (2.25 g, 38.10 mmol) and 1,1″-bis(diphenylphosphino)ferrocenedicholoro palladium (II) dichloromethane complex (280 mg, 0.38 mmol) in DMF (13 ml) was purged with argon and then heated for 18 hours at 100° C. under an argon atmosphere. The cooled reaction mixture was then partitioned between water and DCM, extracted 1×DCM, the combined organic layers dried over sodium sulphate and evaporated. Purification of the residue by normal phase chromatography, eluting with a DCM/methanol gradient, to give the title compound as a yellow oil.

### Step C.1: (S)-2-(3-Bromo-2-fluoro-phenoxymethyl)-tetrahydrofuran

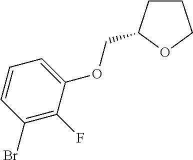 [SMILES: Fc1c(Br)cccc1OC[C@@H]1CCCO1]

Diisopropyl azodicarboxylate (4.42 ml, 22.72 mmol) was added dropwise over 5 minutes to a mixture of 3-bromo-2-fluorophenol (3.10 g, 16.23 mmol), (S)-2-tetrahydrofurylmethanol (2.36 ml, 24.35 mmol), triphenylphosphine (6.81 g, 26.0 mmol) and THF (32 ml) cooled at room temperature. After standing 18 hours at room temperature, volatiles were removed under reduced pressure, the residue partitioned between 1N aqueous sodium hydroxide and DCM, extracted 2× with DCM, the combined organic layers, washed with water, dried over sodium sulphate and evaporated. Purification of the residue by flash column chromatography, eluting with a gradient of ethyl acetate in DCM, gave the title compound as a clear pale-yellow oil. HPLC/MS t_{R} 1.14 min, M+H 294.3 and 292 (Method X). ¹H NMR (400 MHz, CDCl₃) δppm 1.77-2.14 (m, 4H), 3.78-3.84 (m, 1H), 3.86-3.95 (m, 1H), 3.99-4.07 (m, 2H), 4.23-4.33 (m, 1H), 6.87-6.97 (m, 2H), 7.08-7.14 (m, 1H).

### Intermediate D: 1-[2-Fluoro-3-(4,4,5,5-tetramethyl-[1,3,2]dioxaborolan-2-yl)-phenoxymethyl]-7-oxa-bicyclo[2.2.1]heptane

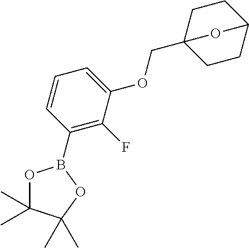 [SMILES: CC1(C)OB(c2cccc(OCC34CCC(CC3)O4)c2F)OC1(C)C]

A mixture of 1-(3-bromo-2-fluoro-phenoxymethyl)-7-oxa-bicyclo[2.2.1]heptane (Step D.1, 2.85 g, 9.46 mmol), bis-(pinacolato)diboron (2.40 g, 9.46 mmol), potassium acetate (2.79 g, 28.4 mmol) and 1,1″-bis(diphenylphosphino)ferrocenedicholoro palladium (II) dichloromethane complex (312 mg, 0.43 mmol) in DMF (10.5 ml) was purged with argon and then heated for 16 hours at 100° C. under an argon atmosphere. The cooled reaction mixture was diluted with ethyl acetate and washed with water then brine, the organic layers dried over Na₂SO₄ and evaporated. Purification of the residue by normal phase column chromatography, eluting with DCM-MeOH (98:2), gave the title compound as orange crystals.

### Step D.1: 1-(3-Bromo-2-fluoro-phenoxymethyl)-7-oxa-bicyclo[2.2.1]heptane

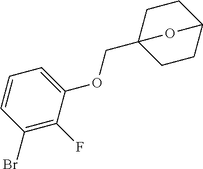 [SMILES: Fc1c(Br)cccc1OCC12CCC(CC1)O2]

To the stirred mixture of 3-bromo-2-fluorophenol (2.25 g, 11.54 mmol), 1-(iodomethyl)-7-oxabicyclo[2.2.1]heptane (Step D.2, 5.15 g, 17.32 mmol and DMF (46 ml) was added tetrabutylammonium iodide (0.213 g, 0.58 mmol) and sodium hydride 60% (0.60 g, 15.0 mmol) and then heated for 16 hours at 100° C. under an argon atmosphere. The cooled reaction mixture was diluted with ethyl acetate and washed with water then brine, the organic layers dried over Na₂SO₄ and evaporated. Purification of the residue by normal phase column chromatography, eluting with ethyl acetate hexane (1:4), gave the title compound as yellow crystals. ¹H-NMR (400 MHz, DMSO-d₆): δppm 7.29-7.18 (m, 2H), 7.10-7.04 (m, 1H), 4.49 (t, 1H), 4.35 (s, 2H), 1.75-1.50 (m, 8H).

### Step D.2: 1-(iodomethyl)-7-oxabicyclo[2.2.1]heptane

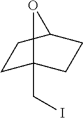

A solution of 4-methylenecyclohexanol (Step D.3, 2.4 g, 21.4 mmol) and N-iodosuccinimide (8.8 g, 37.6 mmol) in dry acetonitrile (100 mL) was stirred at room temperature in the dark overnight. The resulting mixture was poured into water and extracted with ether. The extract was washed successively with sat. aq. Na₂S₂O₄, sat. aq. NaHCO₃ and brine, then dried with Na₂SO₄. After concentration at 200 mbar and 30° C., the residue was purified by silica gel chromatography (EtOAc/Hexane: 0-20 gradient) to afford the title compound. ¹H-NMR (CDCl₃, 400 MHz): 4.66-4.62 (m, 1H), 3.55 (s, 2H), 1.97-1.60 (m, 8H).

### Step D.3: 4-methylenecyclohexanol

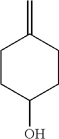

To a solution of 4-methylenecyclohexanone (Step D.4, 2.8 g, 25.45 mmol) in MeOH 100 mL) was added NaBH₄ (1.93 g, 50.9 mmol) at 0° C. The reaction was stirred at room temperature for 2 h, and quenched with sat. aq. NH₄Cl. The reaction was extracted with DCM, the collected organic extracts were dried (Na₂SO₄), concentrated at 200 mbar and 30° C. to afford the title compound which was used without further purification.

### Step D.4: 4-methylenecyclohexanone

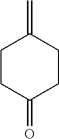

To a solution of 8-methylene-1,4-dioxaspiro[4.5]decane (Step D.5, 5.12 g, 33.2 mmol) in acetone (15 mL) and water (15 mL) was added oxalic acid dihydride (8.33 g, 66.1 mol), the reaction was stirred at room temperature for 3 h. Solid NaHCO₃ was added slowly to the reaction, the solid was filtered and washed thoroughly with diethylether. The combined organic extracts were concentrated at 200 mbar and 30° C. to afford the title compound which was used without further purification. ¹H-NMR (CDCl₃, 400 MHz): 4.88 (s, 2H), 2.52 (t, 4H), 2.43 (t, 4H).

### Step D.5: 8-methylene-1,4-dioxaspiro[4.5]decane

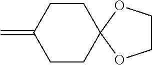

A solution of n-BuLi (2.5 M in hexanes, 30 mL, 75 mmol) was slowly added to a suspension of methyltriphenylphosphonium bromide (28.07 g, 79 mmol) in THF (150 mL) at −10° C. After stirring for 1 h, 1,4-dioxaspiro[4.5]decan-8-one (8.01 g, 51.3 mmol) was added. The reaction was warmed to room temperature and stirred for 4 h. The reaction was quenched with sat. aq. NH₄Cl, extracted by diethyl ether. The combined organic extracts were dried (Na₂SO₄), concentrated at 200 mbar and 30° C. The residue was diluted with DCM and hexanes (1:1), and the solid was filtered. The organic extracts were concentrated at 200 mbar and 30° C., followed by silica gel chromatography (EtOAc/hexanes: 0-10%-20% gradient) to afford the title compound. ¹H-NMR (CDCl₃, 400 MHz): 4.69 (s, 2H), 3.99 (s, 4H), 2.30 (t, 4H), 1.72 (t, 4H).

### Intermediate E: d₉-1-[2-Fluoro-3-(4,4,5,5-tetramethyl-[1,3,2]dioxaborolan-2-yl)-phenoxymethyl]-7-oxa-bicyclo[2.2.1]heptane

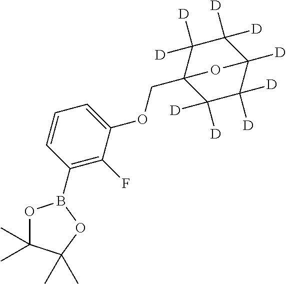 [SMILES: [2H]C1([2H])C([2H])([2H])C2(COc3cccc(B4OC(C)(C)C(C)(C)O4)c3F)OC1([2H])C([2H])([2H])C2([2H])[2H]]

A mixture of d₉-1-(3-bromo-2-fluoro-phenoxymethyl)-7-oxa-bicyclo[2.2.1]heptane (Step E.1, 2.36 g, 7.61 mmol), bis-(pinacolato)diboron (2.32 g, 9.14 mmol), potassium acetate (2.24 g, 22.82 mmol) and 1,1″-bis(diphenylphosphino)ferrocenedicholoro palladium (II) dichloromethane complex (560 mg, 0.77 mmol) in DMF (20 ml) was purged with argon and then heated for 3.5 hours at 100° C. under an argon atmosphere. The cooled reaction mixture was then filtered through celite, partitioned between water and DCM, extracted 1×DCM, the combined organic layers dried over sodium sulphate and evaporated. Purification of the residue by normal phase chromatography, eluting with a ethyl acetate in hexane gradient, to give the title compound as an orange solid. ¹H NMR (400 MHz, CDCl₃) δppm 7.32-7.27 (m, 1H), 7.19-7.12 (m, 1H), 7.06-7.01 (m, 1H), 4.31 (s, 2H), 1.35 (s, 6H), 1.26 (s, 6H).

### Step E.1: d₂-1-(3-Bromo-2-fluoro-phenoxymethyl)-7-oxa-bicyclo[2.2.1]heptane

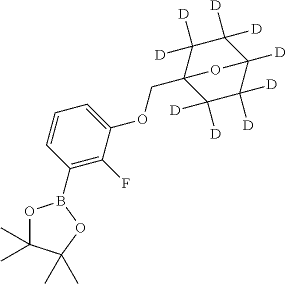 [SMILES: [2H]C1([2H])C([2H])([2H])C2(COc3cccc(B4OC(C)(C)C(C)(C)O4)c3F)OC1([2H])C([2H])([2H])C2([2H])[2H]]

A mixture of d₉-1-iodomethyl-7-oxa-bicyclo[2.2.1]heptane (Step M.2, 17.21 g, 32.7 mmol), 3-bromo-2-fluorophenol (5.0 g, 26.2 mmol), K₂CO₃ (7.24 g, 52.4 mmol) and acetonitrile (6 ml) were heated with stirring in a sealed pressure vessel at 150° C. for 2 days. The cooled reaction mixture was partitioned between water and ethyl acetate, extracting 2× with ethyl acetate, the combined organic layers washed with 1M aqueous NaOH, dried over sodium sulphate and evaporated. Recrystallisation from hexanes gave the title compound as a pale brown crystalline solid. ¹H NMR (400 MHz, CDCl₃) δppm 4.33 (s, 2H), 6.90-6.96 (m, 1H), 6.97-7.04 (m, 1H), 7.09-7.16 (m, 1H).

### Intermediate F: 2-[2-Fluoro-3-(oxetan-2-ylmethoxy)-phenyl]-4,4,5,5-tetramethyl-[1,3,2]dioxaborolane

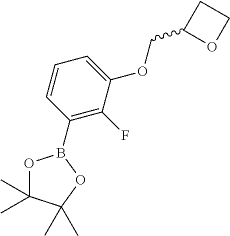 [SMILES: CC1(C)OB(c2cccc(OCC3CCO3)c2F)OC1(C)C]

The title compound was prepared in an analagous fashion to Intermediate C by substituting (S)-2-tetrahydrofurylmethanol with 2-hydroxymethyloxetane.

### Intermediate G: 2-[4-Fluoro-3-(tetrahydro-furan-2-ylmethoxy)-phenyl]-4,4,5,5-tetramethyl-[1,3,2]dioxaborolane

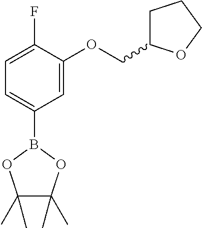 [SMILES: CC1(C)OB(c2ccc(F)c(OCC3CCCO3)c2)OC1(C)C]

2-fluoro-5-(4,4,5,5-tetramethyl-1,3,2-dioxaborolan-2-yl)phenol (Step G.1, 300 mg, 1.260 mmol) and 2-(bromomethyl)tetrahydrofuran (0.43 mL, 624 mg, 3.781 mmol) were dissolved in anhydrous MeCN (3 mL). Solid K₂CO₃ (697 mg, 5.041 mmol) was added. The reaction vessel was sealed and heated at 125° C. for 16 hours. The reaction mixture was cooled to room temperature and the solids removed by filtration. The solids were washed well with additional MeCN. The combined organics were concentrated. The resulting residue was purified with silica gel chromatography (0-20% gradient EtOAc in hexanes) to obtain the title compound as a clear oil. TLC Rf=0.50 (3:1 hexanes/EtOAc). MS m/z 323.2 (M+H+) (Method M). ¹H-NMR (400 MHz, DMSO-d₆) δppm 7.33-7.18 (m, 3H), 4.21-4.13 (m, 1H), 4.07-3.95 (m, 2H), 3.81-3.74 (m, 1H), 3.71-3.64 (m, 1H), 2.02-1.65 (m, 4H), 1.29 (s, 12H).

### Step G.1: 2-fluoro-5-(4,4,5,5-tetramethyl-1,3,2-dioxaborolan-2-yl)phenol

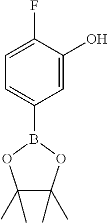 [SMILES: CC1(C)OB(c2ccc(F)c(O)c2)OC1(C)C]

(4-fluoro-3-hydroxyphenyl)boronic acid (850 mg, 5.452 mmol) and pinacol (612 mg, 5.179 mmol) were dissolved in Et₂O (11 mL). The reaction was stirred at room temperature for 17 hours. The reaction mixture was directly applied to a silica gel column. Purification with silica gel chromatography (3:1 hexanes/EtOAc) obtained the title compound as a clear oil which solidified to a waxy white solid upon standing under vacume. TLC Rf=0.55 (3:1 hexanes/EtOAc). MS m/z 239.1 (M+H+) (Method M).

### Intermediate H: 1-[2-Fluoro-5-(4,4,5,5-tetramethyl-[1,3,2]dioxaborolan-2-yl)-phenoxymethyl]-7-oxa-bicyclo[2.2.1]heptane

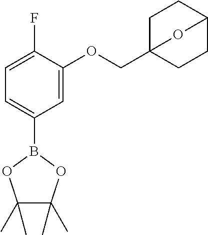 [SMILES: CC1(C)OB(c2ccc(F)c(OCC34CCC(CC3)O4)c2)OC1(C)C]

2-fluoro-5-(4,4,5,5-tetramethyl-1,3,2-dioxaborolan-2-yl)phenol (Step G.1, 300 mg, 1.260 mmol) and 1-(iodomethyl)-7-oxabicyclo[2.2.1]heptane (Step D.2, 900 mg, 3.781 mmol) were dissolved in anhydrous MeCN (3 mL). Solid K₂CO₃ (697 mg, 5.041 mmol) was added. The reaction vessel was sealed and heated at 150° C. for 19 hours. The reaction mixture was cooled to room temperature and the solids removed by filtration. The solids were washed well with additional MeCN. The combined organics were concentrated. The resulting residue was purified with silica gel chromatography (0-20% gradient EtOAc in hexanes) to obtain the title compound as a white solid. TLC Rf=0.5 (4:1 hexanes/EtOAc). MS m/z 349.2 (M+H+) (Method M).

### Intermediate I: 2-[2-Fluoro-5-(tetrahydro-furan-2-ylmethoxy)-phenyl]-4,4,5,5-tetramethyl-[1,3,2]dioxaborolane

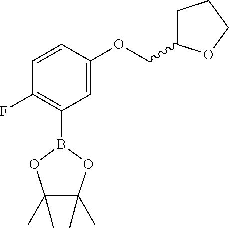 [SMILES: CC1(C)OB(c2cc(OCC3CCCO3)ccc2F)OC1(C)C]

4-fluoro-3-(4,4,5,5-tetramethyl-1,3,2-dioxaborolan-2-yl)phenol (Step I.1, 544 mg, 2.285 mmol) and 2-(bromomethyl)tetrahydrofuran (0.78 mL, 1.131 g, 6.855 mmol) were dissolved in anhydrous MeCN (5.5 mL). Solid K₂CO₃ (1.263 g, 9.141 mmol) was added. The reaction vessel was sealed and heated at 125° C. for 16 hours. The reaction mixture was cooled to room temperature and the solids removed by filtration. The solids were washed well with EtOAc. The combined organics were concentrated. The resulting residue was partially purified with silica gel chromatography (0-20% gradient EtOAc in hexanes) to obtain a −1:1 mixture of the title compound+starting materal (Step I.1) as a clear oil. This mixture was used for subsequent reactions without further purification. MS m/z 323.2+239.1 (M+H+) (Method M).

### Step I.1: 4-fluoro-3-(4,4,5,5-tetramethyl-1,3,2-dioxaborolan-2-yl)phenol

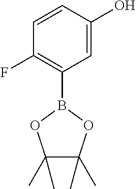 [SMILES: CC1(C)OB(c2cc(O)ccc2F)OC1(C)C]

(2-fluoro-5-hydroxyphenyl)boronic acid (850 mg, 5.452 mmol) and pinacol (612 mg, 5.179 mmol) were dissolved in Et₂O (11 mL). The reaction was stirred at room temperature for 17 hours. The reaction mixture was directly applied to a silica gel column. Purification with silica gel chromatography (3:1 hexanes/EtOAc) obtained the title compound as a clear oil which solidified to a waxy white solid upon standing under vacume. MS m/z 239.1 (M+H+) (Method M).

### Intermediate J: 2-{2-Fluoro-5-[(R)-1-(tetrahydro-furan-2-yl)methoxy]-phenyl}-4,4,5,5-tetramethyl-[1,3,2]dioxaborolane

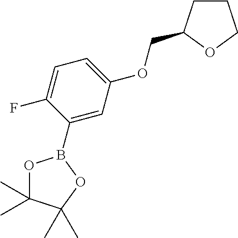 [SMILES: CC1(C)OB(c2cc(OC[C@H]3CCCO3)ccc2F)OC1(C)C]

The title compound was prepared in an analagous fashion to Intermediate K by substituting (S)-2-tetrahydrofurylmethanol with (R)-2-tetrahydrofurylmethanol.

### Intermediate K: 2-{2-Fluoro-5-[(S)-1-(tetrahydro-furan-2-yl)methoxy]-phenyl}-4,4,5,5-tetramethyl-[1,3,2]dioxaborolane

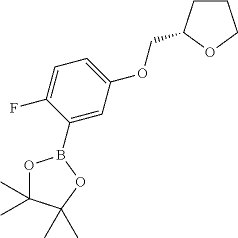 [SMILES: CC1(C)OB(c2cc(OC[C@@H]3CCCO3)ccc2F)OC1(C)C]

A mixture of (S)-2-(3-bromo-4-fluoro-phenoxymethyl)-tetrahydrofuran (Step K.1, 1.1 g, 4.00 mmol), bis-(pinacolato)diboron (1.02 g, 4.00 mmol), potassium acetate (0.71 g, 11.99 mmol) and 1,1″-bis(diphenylphosphino)ferrocenedicholoro palladium (II) dichloromethane complex (71 mg, 0.12 mmol) in DMF (8 ml) was purged with argon and then heated for 2.3 hours at 100° C. under an argon atmosphere. The cooled reaction mixture was then partitioned between water and DCM, extracted 1×DCM, the combined organic layers dried over sodium sulphate and evaporated. Purification of the residue by normal phase chromatography with a DCM/methanol gradient to give the title compound as a brown solid.

### Step K.1: (S)-2-(3-Bromo-4-fluoro-phenoxymethyl)-tetrahydrofuran

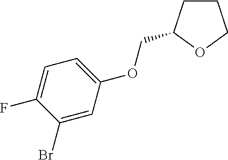 [SMILES: Fc1ccc(OC[C@@H]2CCCO2)cc1Br]

Diisopropyl azodicarboxylate (2.17 ml, 11.18 mmol) was added dropwise over 5 minutes to a mixture of 3-bromo-4-fluorophenol (2.14 g, 11.18 mmol), (S)-2-tetrahydrofurylmethanol (1.3 ml, 13.4 mmol), triphenylphosphine (4.11 g, 15.7 mmol) and THF (22 ml) cooled at room temperature. After standing 18 hours at room temperature, volatiles were removed under reduced pressure, the residue partitioned between 1N aqueous sodium hydroxide and DCM, extracted 2× with DCM, the combined organic layers, washed with water, dried over sodium sulphate and evaporated. Purification of the residue by flash column chromatography, eluting with a gradient of ethyl acetate in DCM, gave the title compound as a clear pale-yellow oil. ¹H NMR (400 MHz, CDCl₃) δppm 1.38-1.59 (m, 1H), 1.78-2.01 (m, 2H), 2.02-2.23 (m, 1H), 3.67-3.74 (m, 1H), 3.77-3.93 (m, 3H), 4.43-4.58 (m, 1H), 6.61-6.72 (m, 1H), 6.93-7.09 (m, 1H), 7.16-7.23 (m, 1H).

### Intermediate L: 1-[4-Fluoro-3-(4,4,5,5-tetramethyl-[1,3,2]dioxaborolan-2-yl)-phenoxymethyl]-7-oxa-bicyclo[2.2.1]heptane

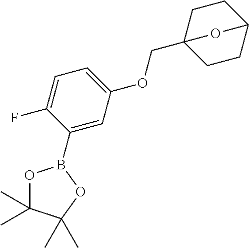 [SMILES: CC1(C)OB(c2cc(OCC34CCC(CC3)O4)ccc2F)OC1(C)C]

A mixture of 1-(3-bromo-4-fluoro-phenoxymethyl)-7-oxa-bicyclo[2.2.1]heptane (Step L.1, 2.60 g, 8.55 mmol), bis-(pinacolato)diboron (2.17 g, 8.55 mmol), potassium acetate (2.52 g, 25.6 mmol) and 1,1″-bis(diphenylphosphino)ferrocenedicholoro palladium (II) dichloromethane complex (188 mg, 0.26 mmol) in DMF (9.5 ml) was purged with argon and then heated for 20 hours at 100° C. under an argon atmosphere. The cooled reaction mixture was diluted with ethyl acetate and washed with water then brine, the organic layers dried over Na₂SO₄ and evaporated. Purification of the residue by normal phase column chromatography, eluting with DCM-MeOH (98:2), gave the title compound as orange oil.

### Step L1: 1-(3-Bromo-4-fluoro-phenoxymethyl)-7-oxa-bicyclo[2.2.1]heptane

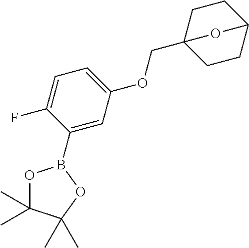 [SMILES: CC1(C)OB(c2cc(OCC34CCC(CC3)O4)ccc2F)OC1(C)C]

To the stirred mixture of 3-bromo-4-fluorophenol (2.25 g, 11.54 mmol), 1-(iodomethyl)-7-oxabicyclo[2.2.1]heptane (Step D.2, 5.15 g, 17.32 mmol and DMF (46 ml) was added tetrabutylammonium iodide (0.213 g, 0.58 mmol) and sodium hydride 60% (0.60 g, 15.0 mmol) and then heated for 16 hours at 100° C. under an argon atmosphere. The cooled reaction mixture was diluted with ethyl acetate and washed with water then brine, the organic layers dried over Na₂SO₄ and evaporated. Purification of the residue by normal phase column chromatography, eluting with ethyl acetate hexane (1:5), gave the title compound as yellow oil. ¹H-NMR (400 MHz, DMSO-d₆): δppm 7.33-7.24 (m, 2H), 7.03-6.98 (m, 1H), 4.46 (t, 1H), 4.24 (s, 2H), 1.73-1.49 (m, 8H).

### Intermediate M: d₉-1-[4-Fluoro-3-(4,4,5,5-tetramethyl-[1,3,2]dioxaborolan-2-yl)-phenoxymethyl]-7-oxa-bicyclo[2.2.1]heptane

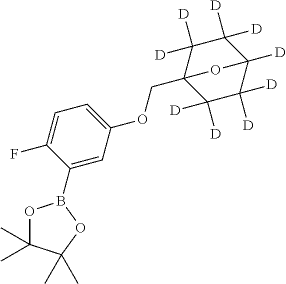 [SMILES: [2H]C1([2H])C([2H])([2H])C2(COc3ccc(F)c(B4OC(C)(C)C(C)(C)O4)c3)OC1([2H])C([2H])([2H])C2([2H])[2H]]

A mixture of d₉-1-(3-bromo-4-fluoro-phenoxymethyl)-7-oxa-bicyclo[2.2.1]heptane (Step M.1, 3.00 g, 9.67 mmol), bis-(pinacolato)diboron (2.52 g, 9.92 mmol), potassium acetate (2.85 g, 29.0 mmol) and 1,1″-bis(diphenylphosphino)ferrocenedicholoro palladium (II) dichloromethane complex (2708 mg, 0.87 mmol) in DMF (11 ml) was purged with argon and then heated for 3.5 hours at 100° C. under an argon atmosphere. The cooled reaction mixture was then filtered through celite, partitioned between water and DCM, extracted 1×DCM, the combined organic layers dried over sodium sulphate and evaporated. Purification of the residue by normal phase chromatography with a ethyl acetate in hexane gradient to give the title compound as an orange oil.

### Step M.1: d₉-1-(3-Bromo-4-fluoro-phenoxymethyl)-7-oxa-bicyclo[2.2.1]heptane

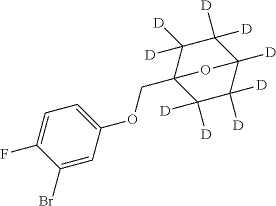 [SMILES: [2H]C1([2H])C([2H])([2H])C2(COc3ccc(F)c(Br)c3)OC1([2H])C([2H])([2H])C2([2H])[2H]]

A mixture of d₉-1-iodomethyl-7-oxa-bicyclo[2.2.1]heptane (Step M.2, 5.43 g, 21.99 mmol), 3-bromo-4-fluorophenol (3.5 g, 18.32 mmol), K₂CO₃ (5.07 g, 36.6 mmol) and acetonitrile (13 ml) were heated with stirring in a sealed pressure vessel at 150° C. for 21 hours. After cooling an additional quantity of d₉-1-iodomethyl-7-oxa-bicyclo[2.2.1]heptane (0.52 g) was added and the sealed vessel heated for a further 24 hours at 150° C. The cooled reaction mixture was partitioned between between water and ethyl acetate, extracting 2× with ethyl acetate, the combined organic layers washed with 1M aqueous NaOH, dried over sodium sulphate and evaporated to give the title compound which was used without further purification. ¹H NMR (400 MHz, CDCl₃) δppm 4.20 (s, 2H), 6.70-6.79 (m, 1H), 7.04 (t, 1H), 7.25-7.31 (m, 1H).

### Step M.2: d₉-1-iodomethyl-7-oxa-bicyclo[2.2.1]heptane

Iodine (170 g, 168 mmol) was added to a mixture of d₉-4-methylene-cyclohexanol (Step M.3, 25.4 g, 168 mmol), sodium carbonate (31.1 g, 293 mmol) and acetonitrile (1.7 l) at room temperature. After stirring for 1 hour at room temperature in the dark the reaction mixture was diluted with 10% aqueous sodium thiosulphate solution, extracted 2× with ethyl acetate, washed with brine, the combined organic layers dried over magnesium sulphate and evaporated under a reduced pressure of 200 mbar at 40° C. to give the title compound as a pale yellow oil. ¹H NMR (400 MHz, CDCl₃) δppm 3.54 (s, 2H).

### Step M.3: d₉-4-methylene-cyclohexanol

Sodium borodeuteride (27.4 g, 654 mmol) was added portion-wise to a solution of d₈-4-methylene-cyclohexanone (Step M.4, 38.68 g, 327 mmol), in THF (82 ml) at 0° C. The reaction mixture was stirred for 1 hour at room temperature, diluted with diethyl ether, washed with water, dried over sodium sulphate and evaporated. Purification of the residue by flash column chromatography, eluting with a gradient of DCM in hexane, gave the title compound as a yellow oil. ¹H NMR (400 MHz, CDCl₃) δppm 4.65 (s, 2H).

### Step M.4: d₈-4-methylene-cyclohexanone

A mixture of d₄-4-methylene-cyclohexanone (Step M.5, 48.0 g, 420 mmol), triethylamine (13.5 ml, 97 mmol), d₁-ethanol (180 ml) and deuterium oxide (20 ml) was stirred for 17 hours at room temperature. The volume was then reduced under a vacuum of 120 mbar at 40° C., then extracted with diethyl ether, the combined organic layers washed with water, dried over magnesium sulphate and evaporated to give a clear yellow oil. Exposure of the isolated material to the above procedure for a second time gave the title compound as a yellow oil. ¹H NMR (400 MHz, CDCl₃) δppm 4.89 (s, 2H).

### Step M.5: d₄-4-methylene-cyclohexanone

Oxalic acid (53.4 g, 594 mmol) was added to a mixture of d₄-8-methylene-1,4-dioxa-spiro[4.5]decane (Step M.6, 43.0 g, 245 mmol), acetone (300 ml) and water (150 ml) at room temperature. After 8 hours sodium bicarbonate was added, the reaction mixture filtered, washing with diethyl ether and the filtrate extracted with diethyl ether. The combined organic layers were then washed with brine, dried over magnesium sulphate and evaporated under a reduced pressure of 200 mbar at 30° C. Exposure of the isolated material to the above procedure for a second time gave the title compound as a colourless oil. ¹H NMR (400 MHz, CDCl₃) δppm 2.39 (s, 4H), 4.89 (s, 2H).

### Step M.6: d₄-8-methylene-1,4-dioxa-spiro[4.5]decane

A solution of n-butyllitium in hexanes (2.5 M, 164 ml, 411 mmol) was added to a mixture of methyltriphenylphosphonium bromide (161 g, 440 mmol) and THF (11) cooled at 10° C. After 85 minutes d₄-1,4-dioxa-spiro[4.5]decan-8-one (Step M.7, 58.9 g, 294 mmol) was added. After stirring for a further 2 hours at room temperature acetone was added followed by partial removal of volatiles under vacuum. Elution of the remaining reaction mixture through a plug of silica gel with a 1:1 mixture of heptane and diethyl ether and evaporation under a reduced pressure of 200 mbar at 35° C. gave the title compound as a clear yellow oil. ¹H NMR (400 MHz, CDCl₃) δppm 1.69 (s, 4H), 3.96 (s, 4H), 4.67 (s, 2H).

### Step M.7: d₄-1,4-dioxa-spiro[4.5]decan-8-one

A mixture of 1,4-dioxa-spiro[4.5]decan-8-one (52 g, 323 mmol), triethylamine (10 ml, 71.7 mmol), d₁-ethanol (152 ml) and deuterium oxide (8 ml) was stirred for 24 hours at room temperature. The volume was then reduced under vacuum at 35° C., benzene added and the mixture evaporated to give a clear yellow oil. Exposure of the isolated material to the above procedure for a second time gave the title compound. ¹H NMR (400 MHz, CDCl₃) δppm 2.00 (s, 4H), 4.16 (s, 4H).

### Intermediate N: 5-bromo-7-[3-(1,1-dioxo-1-thiomorpholin-4-ylmethyl)-cyclobutyl]-7H-pyrrolo[2,3-d]pyrimidin-4-ylamine

A mixture of 5-bromo-4-chloro-7-[cis-3-(1,1-dioxo-1-thiomorpholin-4-ylmethyl)-cyclobutyl]-7H-pyrrolo[2,3-d]pyrimidine (Step N.1, 1.53 g, 3.54 mmol), aqueous ammonium hydroxide solution (25%, 20 ml) and dioxane (20 ml) were heated in a sealed vessel for 17 hours at 100° C. The cooled reaction mixture was then filtered, washing with water, to give the title compound as a white solid. HPLC/MS t_{R} 0.65 min, M+H 414.3 & 416.3 (Method X).

### Step N.1: 5-bromo-4-chloro-7-[cis-3-(1,1-dioxo-1-thiomorpholin-4-ylmethyl)-cyclobutyl]-7H-pyrrolo[2,3-d]pyrimidine

N-Bromosuccinimide (1.12 g, 6.20 mmol) was added to a mixture of 4-chloro-7-[cis-3-(1,1-dioxo-1-thiomorpholin-4-ylmethyl)-cyclobutyl]-7H-pyrrolo[2,3-d]pyrimidine (Step N.2, 2.0 g, 5.64 mmol) in DMF (20 ml) at room temperature. After stirring 4 hours at room temperature the reaction mixture was diluted with DCM, washed with water, then saturated brine, dried over sodium sulphate and evaporated. Purification by flash column chromatography, eluting with a gradient of methanol in DCM, gave the title compound. HPLC/MS t_{R} 1.08 min, M+H 433.3 & 435.3 (Method X).

### Step N.2: 4-chloro-7-[cis-3-(1,1-dioxo-1-thiomorpholin-4-ylmethyl)-cyclobutyl]-7H-pyrrolo[2,3-d]pyrimidine

(4,6-Dichloro-pyrimidin-5-yl)-acetadehyde (1.26 g, 6.37 mmol) was added to a solution of ci33-(1,1-dioxo-1-thiomorpholin-4-ylmethyl)-cyclobutylamine (Step N.3, 1.21 g, 5.54 mmol) in diisopropylethylamine (1.98 ml 11.1 mmol) and ethanol (28 ml) at room temperature and the reaction mixture was then heated for 5 hours at reflux. After cooling to room temperature the reaction mixture was diluted with ethyl acetate, washed with aqueous sodium bicarbonate, then saturated brine, dried over sodium sulphate and evaporated. Purification by flash column chromatography, eluting with a gradient of methanol in DCM, gave the title compound. HPLC/MS t_{R} 0.89 min, M+H 355.4 (Method X).

### Step N.3: cis-3-(1,1-dioxo-1-thiomorpholin-4-ylmethyl)-cyclobutylamine

A suspension of 10% palladium on charcoal (5.40 g) in a solution of [3-(1,1-dioxo-1-thiomorpholin-4-ylmethyl)-cyclobutyl]-carbamic acid benzyl ester (Step N.4, 13 g, 36.9 mmol) in ethanol was stirred under an atmosphere of hydrogen for 4 days. After flushing the apparatus with nitrogen the reaction mixture was filtered and evaporated to give the title compound. ¹H NMR (400 MHz, DMSO) δppm 1.20-1.35 (m, 2H), 1.82-1.99 (m, 1H), 2.20-2.31 (m, 2H), 2.41-2.53 (m, 2H), 2.70-2.86 (m, 4H), 3.02-3.14 (m, 5H).

### Step N.4: [cis-3-(1,1-dioxo-1-thiomorpholin-4-ylmethyl)-cyclobutyl]-carbamic acid benzyl ester

Sodium triacetoxy borohydride (37.6 g, 159 mmol) was added portion-wise to a mixture of (cis-3-formyl-cyclobutyl)-carbamic acid benzyl ester (Step N.5, 12.92 g, 53.2 mmol), thiomorpholine-1,1-dioxide (14.7 g, 106 mmol) and THF (150 ml) at room temperature. After stirring for 2 hours at room tempertature the reaction mixture was partitioned between aqueous sodium bicarbonate solution and DCM, the organic layer washed with water and brine, dried over sodium sulphate and evaporated. Purification of the residue by flash column chromatography, eluting with a gradient of methanol in DCM, gave the title compound. HPLC/MS t_{R} 0.88 min, M+H 353.4 (Method X).

### Step N.5: (cis-3-formyl-cyclobutyl)-carbamic acid benzyl ester

Oxalyl chloride (5.84 ml, 64.1 mmol) in DCM (150 ml) was added dropwise over 15 minutes to a solution of DMSO (11.4 ml, 160 mmol) in DCM (30 ml) cooled at −78° C. After stirring for 20 minutes at 78° C. a solution of (cis-3-Hydroxymethyl-cyclobutyl)-carbamic acid benzyl ester (Step N.6, 12.56 g, 53.4 mmol) in DCM (70 ml) was added dropwise over 15 minutes and 30 minutes later a solution of triethylamine (26.1 ml, 187 mmol) in DCM (30 ml) was added. The reaction mixture was stirred for a further 1 hour at −78° C., allowed to warm to 0° C. over 1 hour and then partitioned between aqueous sodium hydrogen carbonate solution and DCM. The organic layer was washed with water and brine, dried over sodim sulphate and evaporated to give the title compound. HPLC/MS t_{R} 0.92 min, M+H 234.1 (Method X).

### Step N.6: (cis-3-Hydroxymethyl-cyclobutyl)-carbamic acid benzyl ester

An aqueous solution of lithium hydroxide (179 ml, 1 M) was added to a mixture of benzoic acid cis-3-benzyloxycarbonylamino-cyclobutylmethyl ester (Step N.7, 20.2 g, 59.5 mmol) and THF (500 ml) and the reaction mixture stirred for 16 hours at 50° C. After cooling to room temperature the reaction mixture was diluted with ethyl acetate and washed with water followed by brine. The organic layer was then dried over sodium sulphate, evaporated and recrystallised from a mixture of DCM/diethyl ether/heptane to give the title compound. ¹H NMR (400 MHz, DMSO) δppm 1.51-1.64 (m, 2H), 1.88-2.05 (m, 1H), 2.13-2.21 (m, 2H), 3.25-3.36 (m, 3H), 3.76-3.89 (m, 1H), 4.46 (t, 1H), 4.99 (s, 2H), 7.26-7.39 (m, 4H), 7.43-7.52 (m, 1H).

### Step N.7: benzoic acid cis-3-benzyloxycarbonylamino-cyclobutylmethyl ester

Benzyl chloroformate (15.7 ml, 110 mmol) was added dropwise to a mixture of benzoic acid 3-amino-cyclobutylmethyl ester (prepared according to the procedure of: J. Slade Organic Process Research & Development 2007, 11, 825-835., 15 g, 73.1 mmol), diisopropylethylamine (25.5 ml, 146 mmol) and DCM (225 ml) cooled at 0° C. After stirring for 20 hours at room temperature the reaction mixture was diluted with DCM, washed with 5% aqueous potassium hydrogen phosphate solution, aqueous sodium bicarbonate solution, water and brine, dried over sodium sulphate and evaporated. Purification of the residue by flash column chromatography, eluting with a gradient of methanol in DCM, gave the title compound. HPLC/MS t_{R} 1.17 min, M+H 340.1 (Method X).

### Intermediate O: 3-{4-Amino-7-[cis-3-(1,1-dioxo-1-thiomorpholin-4-ylmethyl)-cyclobutyl]-7H-pyrrolo[2,3-d]pyrimidin-5-yl}-4-fluoro-phenol

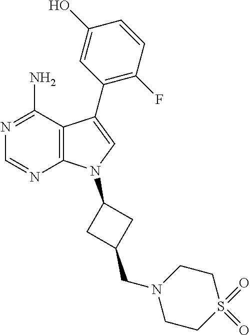 [SMILES: Nc1ncnc2c1c(-c1cc(O)ccc1F)cn2[C@H]1C[C@@H](CN2CCS(=O)(=O)CC2)C1]

A mixture of 5-bromo-7-[cis-3-(1,1-dioxo-thiomorpholin-4-ylmethyl)-cyclobutyl]-7H-pyrrolo[2,3-d]pyrimidin-4-ylamine (Intermediate N, 621 mg, 1.47 mmol), 2-fluoro-5-hydroxyphenylboronic acid (351 mg, 2.20 mmol), potassium carbonate (812 mg, 5.88 mmol) and bis(triphenylphosphine)palladium (II) chloride (103 mg, 0.15 mmol) in DMF (4.5 ml) was purged with argon and then heated for 3.5 hours at 100° C. under an argon atmosphere. The cooled reaction mixture was poured on 1M aqueous NaHCO₃ and extracted with ethyl acetate. The organic layers were washed with water then brine, the organic layers dried over Na₂SO₄ and evaporated. The residue was crystallized from DCM to afford the title compound as slightly yellow crystals. ¹H-NMR (600 MHz, DMSO-d₆): δppm 9.54 (s, 1H), 8.12 (s, 1H), 7.61 (s, 1H), 7.12 (t, 1H), 6.79-6.73 (m, 2H), 6.03 (bs, 2H), 5.08 (m, 1H), 3.08 (m, 4H), 2.91 (m, 4H), 2.69 (d, 2H), 2.56 (m, 2H), 2.30 (m, 1H), 2.17 (m, 2H).

### Intermediate P: 2-(4-Amino-5-iodo-pyrrolo[2,3-d]pyrimidin-7-yl)-(E)-5-oxa-7-aza-spiro[3.4]octan-6-one

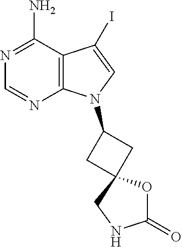 [SMILES: Nc1ncnc2c1c(I)cn2[C@H]1C[C@@]2(CNC(=O)O2)C1]

3-(4-Amino-5-iodo-pyrrolo[2,3-d]pyrimidin-7-yl)-1-aminormethyl-cyclobutanol (Step P.1, 837 mg) was suspended in THF/DMF (5/1) (24 ml) and CDI (453 mg) was added. The reaction mixture was stirred at ambient temperature for 150 minutes. The reaction was left over night in solution. The THF was removed and water (40 ml) was added and a precipitate was obtained which was isolated by filteration to give the title compound as a white solid. HPLC/MS t_{R} 0.55 min, M+H 386.0 (Method X). ¹H NMR (400 MHz, DMSO-d₆) δppm 2.73-2.96 (m, 4H), 3.56 (s, 2H), 5.22 (m, 1H), 6.61 (bs, 1H), 7.53 (s, 1H), 7-71 (s, 1H), 8.08 (s, 1H).

### Step P.1: 3-(4-Amino-5-iodo-pyrrolo[2,3-d]pyrimidin-7-yl)-1-aminomethyl-cyclobutanol

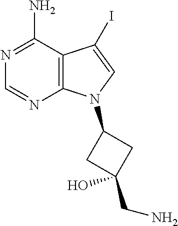 [SMILES: NC[C@]1(O)C[C@H](n2cc(I)c3c(N)ncnc32)C1]

A mixture of 3-(4-amino-5-iodo-pyrrolo[2,3-d]pyrimidin-7-yl)-1-azidomethyl-cyclobutanol (Step P.2, 225 mg, 0.584 mmol), triphenyl phosphine (230 mg, 0.876 mmol), ammonium hydroxide (25%, 0.364 ml, 2.34 mmol), THF (1.3 ml), water (0.33 ml) and methanol (1.3 ml) was stirred for 18 hours at room temperature. The reaction mixture was partitioned between water and ethyl acetate, the combined organic layers dried over sodium sulphate and evaporated. Purification of the residue by flash column chromatography, eluting with a gradient of methanol in DCM containing 1% concentrated ammonia solution, gave the title compound. HPLC/MS t_{R} 0.37 min, M+H 360.1 (Method X). ¹H NMR (400 MHz, DMSO-d₆) δppm 2.16-2.33 (m, 2H), 2.39-2.54 (m, 2H), 2.58 (s, 2H), 5.29 (m, 1H), 6.55 (bs, 1H), 7.66 (s, 1H), 8.06 (s, 1H).

### Step P.2: 3-(4-Amino-5-iodo-pyrrolo[2,3-d]pyrimidin-7-yl)-1-azidomethyl-cyclobutanol

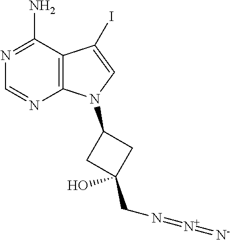 [SMILES: [N-]=[N+]=NC[C@]1(O)C[C@H](n2cc(I)c3c(N)ncnc32)C1]

Sodium azide (212 mg, 3.27 mmol) was added to toluene-4-sulfonic acid 3-(4-amino-5-iodo-pyrrolo[2,3-d]pyrimidin-7-yl)-1-hydroxy-cyclobutylmethyl ester (Step P.3, 420 mg, 0.817 mmol) in DMF (4.1 ml) and the mixture heated for 2 hours at 65° C. Water was added to the cooled reaction mixture to give the title compound as a beige solid which was collected by filtration. MS M+H 386.1. ¹H NMR (400 MHz, DMSO-d₆) δppm 2.23-2.46 (m, 2H), 2.46-2.62 (m, 2H), 3.41 (s, 2H), 5.35 (t, 1H), 5.61 (s, 1H), 6.57 (bs, 1H), 7.72 (s, 1H), 8.07 (s, 1H).

### Step P.3: Toluene-4-sulfonic acid 3-(4-amino-5-iodo-pyrrolo[2,3-d]pyrimidin-7-yl)-1-hydroxy-cyclobutylmethyl ester

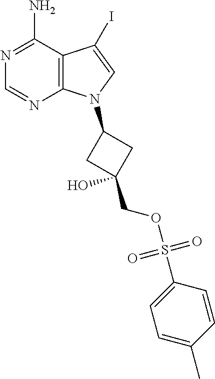 [SMILES: Cc1ccc(S(=O)(=O)OC[C@]2(O)C[C@H](n3cc(I)c4c(N)ncnc43)C2)cc1]

Dibutyltin oxide (615 mg, 2.47 mmol) was added to (E)-3-(4-amino-5-iodo-pyrrolo[2,3-d]pyrimidin-7-yl)-1-hydroxymethyl-cyclobutanol (Intermediate X, 809 mg, 2.25 mmol) in DCM (3 ml), chloroform (6 ml) and methanol (1 ml) and the mixture refluxed for 2.5 hours. After cooling the solvents were removed in vaccuo and DCM (42 ml) added followed by para-toluenesulphonyl chloride (471 mg, 2.47 mmol). After stirring for 18 hours at room temperature water was added, the reaction mixture stirred for a further 30 minutes at room temperature and then evaporated. Trituration with methanol enabled a solid biproduct to be removed and the liquor was evaporated. Purification of the residue by flash column chromatography, eluting with a gradient of methanol in DCM containing 1% concentrated ammonia solution, gave the title compound. HPLC/MS t_{R} 0.87 min, M+H 515.0 (Method X). ¹H NMR (400 MHz, DMSO-d₆) δppm 2.21-2.36 (m, 2H), 2.36-2.57 (m, 2H), 2.40 (s, 3H), 4.06 (s, 1H), 5.26 (t, 1H), 5.57 (s, 1H), 6.58 (bs, 1H), 7.46 (d, 2H), 7.60 (s, 1H), 7.79 (d, 2H), 8.07 (s, 1H).

### Intermediate Q: 5-Iodo-7-[cis-3-(1-oxo-thiomorpholin-4-ylmethyl)-cyclobutyl]-7H-pyrrolo[2,3-d]pyrimidin-4-ylamine

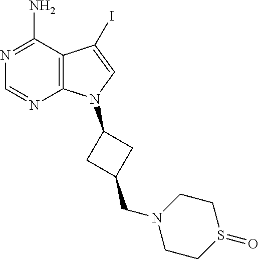 [SMILES: Nc1ncnc2c1c(I)cn2[C@H]1C[C@@H](CN2CCS(=O)CC2)C1]

To the stirred solution of [cis-3-(4-amino-5-iodo-pyrrolo[2,3-d]pyrimidin-7-yl)-cyclobutyl]methanol (Step R.1: 348 mg, 1.0 mmol) and acetonitrile (70 ml) was added 2-iodoxybenzoic acid (IBX, Atlantic SciTech 86900: 561 mg, 2.0 mmol). The reaction mixture was stirred for 1 h at 80° C. The reaction mixture was filtered at 40° C. and the filtrate was concentrated. To the residue was added subsequently DCM (50 ml), diisopropylethylamine (3.43 ml, 20 mmol), 1-oxo-thiomorpholine hydrochloride (312 mg, 2.0 mmol) and sodium triacetoxyborohydride (637 mg, 3.0 mmol) with stirring at rt. The reaction mixture was stirred for 1 h at rt and then partitioned between 1M NaHCO₃ and EtOAc. The combined organic layers were washed with water and brine, dried (Na₂SO₄), filtered and concentrated. The residue was purified by silica gel column chromatography (DCM/MeOH/NH₃^{aq}, 200:20:1) to afford 238 mg of the title compound as pale yellow crystals: HPLC-MS: M+H=446.2 (Rₜ=0.41) (Method X); TLC; R_{f}=0.26 (DCM/MeOH/NH₃^{aq}, 200:20:1).

### Intermediate R: (cis-3-{4-Amino-5-[3-(7-oxa-bicyclo[2.2.1]hept-1-ylmethoxy)-phenyl]-pyrrolo[2,3-d]pyrimidin-7-yl}-cyclobutyl)-methanol

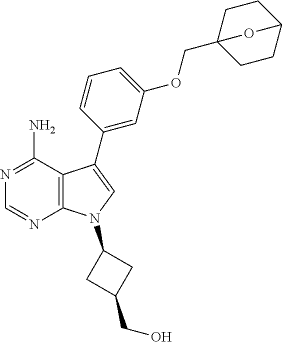 [SMILES: Nc1ncnc2c1c(-c1cccc(OCC34CCC(CC3)O4)c1)cn2[C@H]1C[C@@H](CO)C1]

A mixture of 2-(3-(7-oxabicyclo[2.2.1]heptan-1-ylmethoxy)phenyl)-4,4,5,5-tetramethyl-1,3,2-dioxaborolane (Step R.5, 155 mg, 0.47 mmol), [3-(4-amino-5-iodo-pyrrolo[2,3-d]pyrimidin-7-yl)-cyclobutyl]-methanol (Step R.1, 154 mg, 0.45 mmol), tetrakis(triphenylphosphine)-palladium(0) (52 mg, 0.05 mmol), sodium carbonate (99 mg, 0.94 mmol), water (2 ml) and DMF (4 ml) was heated at 80° C. for 16 hours under an argon atmosphere in the dark. After cooling water was added and the mixture extracted 3× with DCM, dried over sodium sulphate and the organic layers evaporated. Purification of the residue by flash chromatography, eluting with a gradient of methanol in DCM, gave the title compound. HPLC/MS t_{R} 0.91 min, M+H 421.1 (Method X); ¹H-NMR (CDCl₃, 400 MHz): 8.31 (s, 1H), 7.37 (t, 1H), 7.12 (s, 1H), 7.09-7.05 (m, 2H), 6.98 (dd, 1H), 5.29 (broad s, 1H), 5.15-5.09 (m, 1H), 4.61 (t, 1H), 4.30 (s, 2H), 3.73 (d, 2H), 2.70-2.58 (m, 4H), 2.52-2.44 (m, 1H), 1.93-1.78 (m, 4H), 1.67-1.57 (m, 4H).

### Step R.1: [cis-3-(4-amino-5-iodo-pyrrolo[2,3-d]pyrimidin-7-yl)-cyclobutyl]-methanol

The title compound is prepared as described in WO 2005/097800. Or alternatively as described below:

A mixture of [3-(4-chloro-5-iodo-pyrrolo[2,3-d]pyrimidin-7-yl)-cyclobutyl]-methanol (Step R.2, 2.0 g, 5.50 mmol), 25% aqueous ammonia solution (10.4 ml) and 1,4-dioxane (5 ml) were heated in sealed tube at 80° C. for 15.5 hours. After cooling the reaction mixture was evaporated and purified by flash column chromatography, eluting with a gradient of DCM/methanol, to give the title compound. ¹H-NMR (d₆-DMSO, 400 MHz): 8.06 (s, 1H), 7.68 (s, 1H), 6.57 (broad s, 2H), 5.06-4.87 (m, 1H), 4.57 (t, 1H), 3.49-3.40 (m, 2H), 2.45-2.35 (m, 2H), 2.28-2.13 (m, 2H).

### Step R.2: [cis-3-(4-Chloro-5-iodo-pyrrolo[2,3-d]pyrimidin-7-yl)-cyclobutyl]-methanol

A solution of DIBAL-H in toluene (0.73 ml, 0.73 mmol) was added dropwise to a stirred suspension of benzoic acid 3-(4-chloro-5-iodo-pyrrolo[2,3-d]pyrimidin-7-yl)-cyclobutylmethyl ester (Step R.3, 170 mg, 0.36 mmol) in DCM (3 ml) cooled with a dry-ice/acetone bath. After 30 minutes the reaction mixture was warmed over 1 hour to 0° C., stirred 1 hour at 0° C., and silica gel (2 g) was added. The reaction mixture was evaporated and the residue purified by flash chromatography, to give the title compound. HPLC/MS t_{R} 1.09 min, M+H 365.8 (Method X).

### Step R.3: benzoic acid cis-3-(4-chloro-5-iodo-pyrrolo[2,3-d]pyrimidin-7-yl)-cyclobutylmethyl ester

A mixture of benzoic acid 3-(4-chloro-pyrrolo[2,3-d]pyrimidin-7-yl)-cyclobutylmethyl ester (Step R.4, 1.7 g, 4.97 mmol), N-iodosuccinimide (1.23 g, 5.47 mmol) and DMF (9 ml) was stirred at room temperature for 48 hours. Ethyl acetate and water were added and the title compound collected by filtration. HPLC/MS t_{R} 3.46 min, M+H 468.2 and M−H 467.0 (Method Y).

### Step R.4: benzoic acid cis-3-(4-chloro-pyrrolo[2,3-d]pyrimidin-7-yl)-cyclobutylmethyl ester

A mixture of (4,6-dichloro-pyrimidin-5-yl)-acetaldehyde (Astatech, 1.40 g, 7.31 mmol), benzoic acid 3-amino-cyclobutylmethyl ester (prepared as described in Org. Process Res. Dev. 2007, 11, 825-835., 1.5 g, 7.31 mmol), diisopropylethylamine (0.95 g, 7.31 mmol) and ethanol (15 ml) were heated at reflux for 5.5 hours under an argon atmosphere. The reaction mixture was evaporated, taken up in THF (10 ml), aqueous HCl (4 ml, 4M) added and stood at room temperature for 1 hour. The volume of the mixture was then reduced under vacuum, made neutral with aqueous sodium bicarbonate solution, extracted 3× with DCM, the organic layers dried over sodium sulphate and evaporated. Purification by flash column chromatography, eluting with a DCM/EtOAc gradient gave the title compound. HPLC/MS t_{R} 1.52 min, M+H 342.1 (Method X).

### Step R.5: 2-(3-((7-oxabicyclo[2.2.1]heptan-1-ylmethoxy)phenyl)-4,4,5,5-tetramethyl-1,3,2-dioxaborolane

A mixture of 1-(iodomethyl)-7-oxabicyclo[2.2.1]heptanes (Step D.2, 2.24 g, 9.4 mmol), 3-(4,4,5,5-tetramethyl-1,3,2-dioxaborolan-2-yl)phenol (4.14 g, 18.8 mmol) and K₂CO₃ (5.19 g, 37.6 mmol) in dry acetonitrile (24 mL) were heated at 150° C. in a pressure vessel for 18 h. The reaction was cooled to room temperature and filtered, the filtrate was concentrated in vacuo, followed by silica gel chromatography (EtOAc/hexanes: 1-20% gradient) to afford the title compound. ¹H-NMR (CDCl₃, 400 MHz): 7.42-7.36 (m, 2H), 7.29 (t, 1H), 7.11-7.06 (m, 1H), 4.64-4.59 (m, 1H), 4.29 (s, 2H), 1.94-1.75 (m, 4H), 1.65-1.56 (m, 4H), 1.34 (s, 12H).

### Intermediate S: 1-{4-[3-(4-Amino-5-iodo-pyrrolo[2,3-d]pyrimidin-7-yl)-cyclobutyl]-piperazin-1-yl}-ethanone

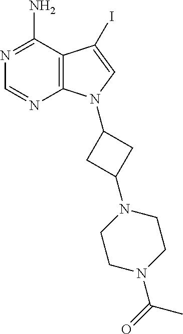 [SMILES: CC(=O)N1CCN(C2CC(n3cc(I)c4c(N)ncnc43)C2)CC1]

Sodium triacetoxyborohydride (552 mg, 2.61 mmol) was added portionwise over 5 minutes to a mixture of 3-(4-amino-5-iodo-pyrrolo[2,3-d]pyrimidin-7-yl)-cyclobutanone (Step S.1, 570 mg, 1.73 mmol), N-acetylpiperazine (267 mg, 2.09 mmol), acetic acid (313 mg, 5.21 mmol) and 1,2-dichloroethane (4 ml) at room temperature. After stirring for 1 hour at room temperature the reaction mixture was partitioned between water and DCM, extracting 5× with DCM, the combined organic layers dried over sodium sulphate and evaporated to give the title compound as a pale-beige solid. HPLC/MS t_{R} 0.64 min, M+H 441.0 (Method X).

### Step S.1: 3-(4-amino-5-iodo-pyrrolo[2,3-d]pyrimidin-7-yl)-cyclobutanone

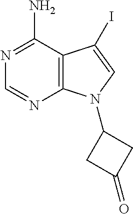 [SMILES: Nc1ncnc2c1c(I)cn2C1CC(=O)C1]

Sodium periodate (7.1 g, 33.3 mmol, 1.5 eq) was added to a stirred suspension of 3-(4-amino-5-iodo-pyrrolo[2,3-d]pyrimidin-7-yl)-1-hydroxymethyl-cyclobutanol (Intermediate V, 8 g, 22.2 mmol) in 400 mL of THF/H₂O (3/1, v:v). the reaction mixture was stirred for 18 h at rt, diluted with ethyl acetate/NaHCO₃^{sat} and extracted with ethyl acetate. The combined organic extracts were washed with H₂O and brine, dried (Na₂SO₄), filtered and concentrated. The residue was purified by silica gel column chromatography (DCM/MeOH, 95:5) followed by trituration in diethyl ether to afford 4.3 g of the title compound as a yellow solid: ES-MS: 329.1 [M+H]⁺; R_{f}=0.17 (DCM/MeOH, 95:5).

### Intermediate T: 1-{4-[cis-3-(4-Amino-5-iodo-pyrrolo[2,3-d]pyrimidin-7-yl)-cyclobutyl]-piperazin-1-yl}-ethanone

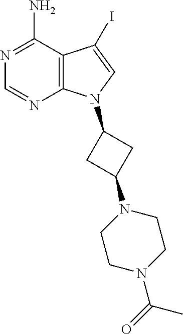 [SMILES: CC(=O)N1CCN([C@H]2C[C@@H](n3cc(I)c4c(N)ncnc43)C2)CC1]

Sodium triacetoxyborohydride (707 mg, 3.34 mmol) was added portionwise over 5 minutes to a mixture of 3-(4-amino-5-iodo-pyrrolo[2,3-d]pyrimidin-7-yl)-cyclobutanone (Step S.1, 730 mg, 2.23 mmol), N-acetylpiperazine (342 mg, 2.67 mmol), acetic acid (401 mg, 6.67 mmol) and 1,2-dichloroethane (4.5 ml) at room temperature. After stirring for 3.5 hours at room temperature the reaction mixture was diluted with aqueous sodium hydrogen carbonate solution (10 ml), stirred for a further 10 minutes and then filtered, washing with water, to give the title compound as a beige solid after drying at 60 QC under high vacuum. ¹H NMR (400 MHz, CDCl₃) δppm 1.96 (s, 3H), 2.21-2.33 (m, 6H), 2.44-2.66 (m, 3H), 3.38-3.44 (m, 4H), 4.76-4.84 (m, 1H), 6.57 (s, br, 2H), 7.61 (s, 1H), 8.07 (s, 1H).

### Intermediate U: 5-Iodo-7-[cis-3-(1-oxo-thiomorpholin-4-yl)-cyclobutyl]-7H-pyrrolo[2,3-d]pyrimidin-4-ylamine

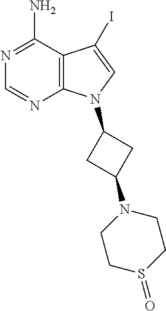 [SMILES: Nc1ncnc2c1c(I)cn2[C@H]1C[C@@H](N2CCS(=O)CC2)C1]

To the stirred solution of 3-(4-amino-5-iodo-pyrrolo[2,3-d]pyrimidin-7-yl)-cyclobutanone

(Step S.1; 680 mg, 2.05 mmol), 1,2-dichloroethane (55 ml) and diisopropylethylamine (1.79 ml, 10.25 mmol) was subsequently added 1-oxo-thiomorpholine hydrochloride (638 mg, 4.10 mmol) and sodium triacetoxyborohydride (652 mg, 3.08 mmol) at 0° C. The reaction mixture was stirred for 1 h at room temperature, and then poured into the stirred mixture of water (150 ml) and EtOAc (150 ml). The precipitate was filtered and washed with water and EtOAc. The solid collected was dried in vacuo to afford the title compound as beige crystals. HPLC-MS: M+H=432.1 (Rₜ=0.43); TLC; R_{f}=0.36 (DCM/MeOH/NH₃^{aq}, 200:20:1).

### Intermediate V: 3-(4-Amino-5-iodo-pyrrolo[2,3-d]pyrimidin-7-yl)-1-hydroxymethyl-cyclobutanol

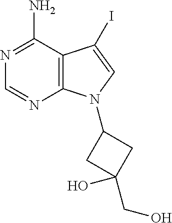 [SMILES: Nc1ncnc2c1c(I)cn2C1CC(O)(CO)C1]

A 2:1 E- to Z mixture of 3-(4-chloro-5-iodo-pyrrolo[2,3-d]pyrimidin-7-yl)-1-hydroxymethyl-cyclobutanol (Step V.1, 6 g, 15.8 mmol) was suspended in a mixture of dioxane (30 mL) and aqueous NH₃ (25%, 60 mL) and transferred into three glass autoclaves vessels (50 mL) (Büchi, Flawil) and stirred at 100° C. for 19 hours. The combined reaction mixtures were concentrated under reduced pressure to give the crude reaction mixture of intermediates Q and R. This isolated mixture of both isomers was used for the next reaction step without further purification. MS (Method L) M+H=361 (100%). HPLC (Method B): t_{R} 1.83 minutes. TLC (NH₃/MeOH/CH₂Cl₂=1:10:89): R^{F}=0.33 and 0.31, ¹H-NMR (600 MHz, DMSO-d₆): δppm (peak intensities Z/E=1:2) 8.08 (E)/8.07 (Z) (s/s, 1H), 7.74 (4/7.58 (E) (s/s, 1H), 6.65 (s/broad, 2H), 5.30/4.80 (t/t, 1H), 5.26/5.13 (s/s, 1H), 4.84 (m, 1H), 3.30 (m, 2H), 2.70/2.30 (t/t, 2H, Z-isomer), 2.60/2.25 (t/t, 2H, E-isomer).

### Step V.1: E- and Z-3-(4-Chloro-5-iodo-pyrrolo[2,3-d]pyrimidin-7-yl)-1-hydroxymethyl-cyclobutanol

3-(4-Chloro-pyrrolo[2,3-d]pyrimidin-7-yl)-1-hydroxymethyl-cyclobutanol (Step V.2, 6.94 g, 27.4 mmol) and NIS (7.39 g, 32.8 mmol) was dissolved in DMF (110 mL) and the mixture stirred at 60° C. under argon. After 2.5 hours, NIS (0.25 g, 1.1 mmol) was added and the reaction mixture stirred for a further 1 hour at 60° C. After concentration of the reaction mixture under reduced pressure, sodium bicarbonate solution (15 mL) was added and the resulting suspension was extracted with AcOEt (30 mL, 8×). The combined organic phases were washed with Na₂SO₃ solution (10 mL, 2×) and brine (5 mL, 2×), dried (MgSO₄), and concentrated under reduced pressure to give a beige solid, which was further suspended in hexane and washed, and then dried under vacuum to give the title compound as a beige solid. NS (Method L) M+H=380/382. HPLC (Method B): t_{R} 2.53 min. ¹H-NMR (600 MHz, DMSO-d₆): δppm (peak intensities Z/E=1:2) 8.61 (E)/8.59 (Z) (s/s, 1H), 8.25 (4/8.12 (E) (s/s, 1H), 5.40/4.86 (quint/quint, 1H), 5.29/5.16 (s/s, 1H), 4.80 (m, 1H), 3.39/3.30 (d/d, 2H), 2.70/2.30 (t/t, 2H/Z-isomer), 2.60/2.25 (t/t, 2H/E-isomer).

### Step V.2: 3-(4-Chloro-pyrrolo[2,3-d]pyrimidin-7-yl)-1-hydroxymethyl-cyclobutanol

(2,4-Dichloro-pyrimidin-5-yl)-acetaldehyde (7.21 g, 37.7 mmol), 3-amino-1-hydroxymethyl-cyclobutanol (Step V.3, 4.42 g, 37.7 mmol), and DIPEA (13.18 mL, 75 mmol) were dissolved in EtOH (190 mL) and stirred under reflux (oil bath at 90° C.) for 4.5 hours. After cooling to room temperature, TFA (260 mmol, 20 mL) was added and the reaction mixture stirred under reflux for a further 1 hour. After cooling to room temperature, conc. NaHCO₃ solution (0.5 L) was added, the alcohol evaporated under reduced pressure, and the reaction mixture was then extracted with AcOEt (4×, 100 mL). The combined organic phases were washed with conc. NaHCO₃ solution (50 mL) and brine (40 mL), dried (MgSO₄), concentrated under reduced pressure, purified by normal phase chromaography on a 120 g RediSept silica gel column, and fractioned by means of a Sepacore Control chromatography system (Büchi, Flawil, Switzerland) (eluent: 1 to 10% MeOH (10% NH3) in CH₂Cl₂) to give the title compound as a beige solid. MS (Method L) M+H=254/256 (100%). HPLC (Method B): t_{R} 2.24 min. ¹H-NMR (600 MHz, DMSO-d₆): δppm (peak intensities Z/E=1:2) 8.63 (E)/8.60 (Z) (s/s, 1H), 8.02 (4/7.89 (E) (d/d, 1H), 6.72 (Z)/6.68 (E) (d/d, 1H), 3.41/2.78 (quint/quint, 1H), 3.30 (S/broad, 4H), 3.21/3.14 (d/d, 2H), 2.29/1.50 (m/m, 2H/Z-isomer), 1.95/1.70 (t/t, 2H/E-isomer).

### Step V.3: 3-Amino-1-hydroxymethyl-cyclobutanol

(3-Hydroxy-3-hydroxymethyl-cyclobutyl)-carbamic acid benzyl ester (Step V.4, 9.49 g, 37.8 mmol) was dissolved in THF/MeOH (1:1, 150 mL) and hydrogenated under 1 atmosphere of hydrogen for 1 hour in the presence of Pd/C Engelhard 4505 (1.5 g). The reaction mixture was then filtered and the solvent evaporated under reduced pressure to give the title compound as a brown oil. ¹H-NMR (400 MHz, DMSO-d₆): δppm (peak intensities Z/E=1:2) 5.40/4.86 (quint/quint, 1H), 5.29/5.16 (s/s, 1H,), 4.85 (m, 1H), 3.41/3.31 (d/d, 2H), 2.75/2.40 (t/t, 2H/Z-isomer), 2.68/2.30 (t/t, 2H/E-isomer).

### Step V.4: (3-Hydroxy-3-hydroxymethyl-cyclobutyl)-carbamic acid benzyl ester

(3-Methylene-cyclobutyl)-carbamic acid benzyl ester (Step V.5, 9.915 g, 45.6 mmol) dissolved in tert.-butanol/H₂O (40 mL, 1:1) was added to a solution of AD-Nix alpha (70 g, 50.2 mmol) in tert.-butanol/H₂O (360 mL, 1:1) at 0° C. After stirring at room temperature for 16 hours, the reaction mixture was cooled to 0° C. and Na₂SO₃ (40 g) was added and the reaction mixture was stirred for a further 1 hour at room temperature. After adding H₂O (150 mL), the reaction mixture was extracted with AcOEt (150 mL, 3×). The combined organic phases were washed with brine, dried (MgSO₄), and concentrated under reduced pressure to give the title compound as white solid. MS (Method L): M+H=252. HPLC (Method B): t_{R} 2.32 minutes. TLC (NH₃/MeOH/CH₂Cl₂=1:10:89): R^{F}=0.25. ¹H-NMR (600 MHz, DMSO-d₆): δppm (peak intensities Z/E=1:2) 7.51 (4/7.44 (E) (s/s, 1H), 7.35 (m, 5H), 4.95 (s, 2H), 4.80 (Z)/4.70 (E) (s/s, 1H), 4.65/4.62 (t/t, 1H), 4.12 (E)/3.52 (Z) (sextet/sextet, 1H), 3.25 (4/3.20 (E) (d/d, 2H), 2.30/1.80 (t/t, 2H/Z-isomer), 1.96 (t, 2H/E-isomer).

### Step V.5: (3-Methylene-cyclobutyl)-carbamic acid benzyl ester

Diphenylphosphoryl azide (25.3 g, 89 mmol) was added to 3-methylene cyclobutyl carboxylic acid (10 g, 89 mmol) and NEt₃ (15 mL, 105 mmol) dissolved in dioxane/MeCN (15 mL/35 mL) over 15 minutes. The temperature of the reaction mixture then increased to 75° C. with the evolution of gas. After heating the reaction mixture for a further 1 hour at 100° C., benzyl alcohol (20 mL) was added and the reaction mixture was then stirred for 19 hours at 100° C. After cooling and evaporation of the solvent, the residue was taken up in AcOEt (250 mL) and extracted with half conc. NH₄Cl solution (80 mL), half con. NaHCO₃ solution (80 mL), and brine (40 mL), dried (MgSO₄), and concentrated under vacuum. The residue was purified by means of a 120 g RediSept silica gel column using a Sepacore Control chromatographic separator (Büchi) (eluent: hexane/AcOEt=1:9 to 4:6) to give the title compound as a white solid. MS (Method L) M+H=218. HPLC (Method B): t_{R} 3.12 minutes. TLC (AcOEt/hexane=1:4): R^{F}=0.30. ¹H-NMR (400 MHz, DMSO-d₆): δppm 7.64 (d, 1H), 7.32 (m, 5H), 4.99 (s, 2H), 4.76 (s, 2H), 3.95 (sextet, 1H), 2.85 (m, 2H), 2.62 (m, 2H).

### Intermediate W: 5-Iodo-7-(3-thiomorpholin-4-yl-cyclobutyl)-7H-pyrrolo[2,3-d]pyrimidin-4-ylamine

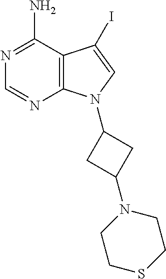 [SMILES: Nc1ncnc2c1c(I)cn2C1CC(N2CCSCC2)C1]

Sodium triacetoxyborohydride (242 mg, 1.14 mmol) was added portionwise over 5 minutes to a mixture of 3-(4-amino-5-iodo-pyrrolo[2,3-d]pyrimidin-7-yl)-cyclobutanone (Step S.1, 250 mg, 0.762 mmol), thiomorpholine (0.086 ml, 0.914 mmol), acetic acid (0.218 ml, 3.81 mmol) and 1,2-dichloroethane (3 ml) at 0° C. After warming to room temperature and stirring for 2 hours at room temperature the reaction mixture was diluted with water (10 ml), extracted with DCM, the combined organic layers dried over sodium sulphate and evaporated to give the title compound as an orange solid. ¹H NMR (400 MHz, DMSO-d₆) δppm 2.11-2.27 (m, 2H), 2.50-2.70 (m, 11H), 4.69-4.86 (m, 1H), 6.57 (s, br, 2H), 7.59 (s, 1H), 8.06 (s, 1H).

### Intermediate X and Y: (E)-3-(4-amino-5-iodo-pyrrolo[2,3-d]pyrimidin-7-yl)-1-hydroxymethyl-cyclobutanol and (Z)-3-(4-amino-5-iodo-pyrrolo[2,3-d]pyrimidin-7-yl)-1-hydroxymethyl-cyclobutanol

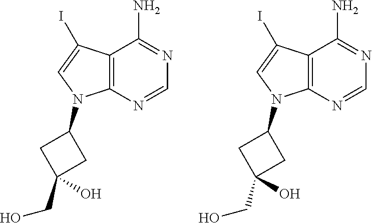 [SMILES: Nc1ncnc2c1c(I)cn2[C@H]1C[C@@](O)(CO)C1 ; Nc1ncnc2c1c(I)cn2[C@H]1C[C@](O)(CO)C1]

The mixture (2:1) of the geometric isomers E- and Z-3-(4-amino-5-iodo-pyrrolo[2,3-d]pyrimidin-7-yl)-1-hydroxymethyl-cyclobutanol (Intermediate V: 34.5 g, 82.5 mmol) was recrystallized several times from methanol to afford Intermediate X (E-isomer) as yellow crystals. The mother liquors were combined, concentrated and dried in vacuo to afford the enriched Intermediate Y (Z-isomer) as beige crystals.

Alternatively, recrystallisation of the E:Z mixture (Intermediate V) from acetic acid gives (E)-3-(4-amino-5-iodo-pyrrolo[2,3-d]pyrimidin-7-yl)-1-hydroxymethyl-cyclobutanol (Intermediate X) as a white solid. ¹H NMR (400 MHz, DMSO) δppm 8.06 (s, 1H), 7.56 (s, 1H), 6.57 (s, br, 2H), 5.29 (pent, 1H), 5.06 (s, 1H), 4.84 (t, 1H), 3.27 (d, 2H), 2.58-2.50 (m, 2H), 2.26-2.19 (m, 2H).

### Intermediate Z: (±)-7-(cis-4-aminocyclohexyl)-5-(2-fluoro-5-((tetrahydrofuran-2-yl)methoxy)phenyl)-7H-pyrrolo[2,3-d]pyrimidin-4-amine

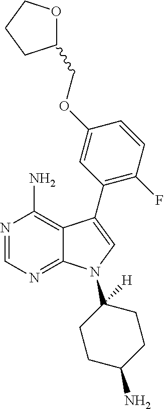

To a solution of (±)-7-(cis-4-azidocyclohexyl)-5-(2-fluoro-5-((tetrahydrofuran-2-yl)methoxy)phenyl)-7H-pyrrolo[2,3-d]pyrimidin-4-amine (Step Z.1, 41 mg, 0.09 mmol) in 2.0 mL of THF was added Ph₃P (45 mg, 0.18 mmol) and aqueous NaOH (0.1 N in water, 0.3 mL, 0.03 mmol) sequentially. The reaction mixture was stirred at room temperature overnight. The reaction mixture was partitioned between DCM and water and the organic extracts were dried (Na₂SO₄). After concentration, the residue was purified with silica gel chromatography (0-10% gradient MeOH in DCM) to obtain the title compound. MS m/z 426.2 (M+H+) (Method M).

### Step Z.1: (±)-7-(cis-4-azidocyclohexyl)-5-(2-fluoro-5-((tetrahydrofuran-2-yl)methoxy)phenyl)-7H-pyrrolo[2,3-d]pyrimidin-4-amine

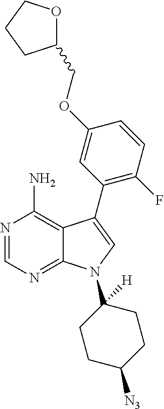

To a solution of (±)-(trans-4-(4-amino-5-(2-fluoro-5-((tetrahydrofuran-2-yl)methoxy)phenyl)-7H-pyrrolo[2,3-d]pyrimidin-7-yl)cyclohexyl methanesulfonate (Step Z.2, 45 mg, 0.09 mmol) in DMF (2.0 mL) was added sodium azide (6.4 mg, 0.1 mmol). The reaction mixture was heated at 80° C. for 16 h. The mixture was cooled to room temperature and then partitioned between EtOAc and water. The collected organic extracts were dried (Na₂SO₄). After concentration, the residue was purified with silica gel chromatography (0-10% gradient MeOH in DCM) to obtain the title compound. MS m/z 452.2 (M+H+) (Method M).

### Step Z.2: (±)-trans-4-(4-amino-5-(2-fluoro-5-((tetrahydrofuran-2-yl)methoxy)phenyl)-7H-pyrrolo[2,3-d]pyrimidin-7-yl)cyclohexyl methanesulfonate

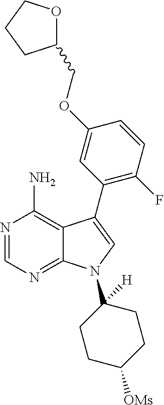

To a solution of (±)-trans-4-(4-amino-5-(2-fluoro-5-((tetrahydrofuran-2-yl)methoxy)phenyl)-7H-pyrrolo[2,3-d]pyrimidin-7-yl)cyclohexanol (Step Z.3, 40 mg, 0.09 mmol) in DCM (1.0 mL) was sequentially added methylsulfonyl chloride (0.01 mL, 0.13 mmol) and triethylamine (0.03 mL, 0.27 mmol). The reaction was stirred at room temperature 30 min. and then partitioned between DCM and water. The collected organic extracts were dried (Na₂SO₄). Concentrated in vacuo obtained the title compound without further purification. MS m/z 505.2 (M+H+) (Method M).

### Step Z.3: (±)-trans-4-(4-amino-5-(2-fluoro-5-((tetrahydrofuran-2-yl)methoxy)phenyl)-7H-pyrrolo[2,3-d]pyrimidin-7-yl)cyclohexanol

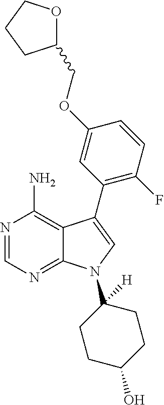

A mixture of (±)-2-[2-fluoro-5-(tetrahydro-furan-2-ylmethoxy)-phenyl]-4,4,5,5-tetramethyl-[1,3,2]dioxaborolane (Intermediate I, 35 mg, 0.1 mmol), trans-4-(4-amino-5-iodo-7H-pyrrolo[2,3-d]pyrimidin-7-yl)cyclohexanol (Step Z.4, 26 mg, 0.072 mmol) and Na₂CO₃ (15 mg, 0.144 mmol) in THF (2.0 ml) and water (0.5 ml) was purged with nitrogen. Tetrakis(triphenylphosphine)palladium (4.2 mg, 0.004 mmol) was added. The reaction vessel was sealed under nitrogen and heated under microwave irradiation for 10 minutes at 120° C. The cooled reaction mixture was diluted with ethyl acetate and then sequentially washed with water and brine. The collected organic extracts were dried (Na₂SO₄). After concentration, the residue was purified with silica gel chromatography (0-5% gradient MeOH in DCM) to obtain the title compound. MS rink 427.2 (M+H+) (Method M).

### Step Z.4: trans-4-(4-amino-5-iodo-7H-pyrrolo[2,3-d]pyrimidin-7-yl)cyclohexanol

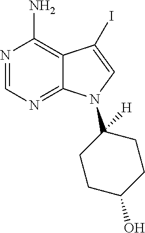

A mixture of trans-4-(4-chloro-5-iodo-7H-pyrrolo[2,3-d]pyrimidin-7-yl)cyclohexanol (Step Z.5, 1.90 g, 5.0 mmol), aqueous ammonium hydroxide solution (25%, 10 mL) and dioxane (10 mL) was heated in a sealed vessel for 17 hours at 100° C. The reaction mixture was cooled and the resulting product precipitate was collected by filtration. This collected solid was washed with water to obtain the title compound as a white solid without further purification. MS m/z 359.0 (M+H+) (Method M).

### Step Z.5: trans-4-(4-chloro-5-iodo-7H-pyrrolo[2,3-d]pyrimidin-7-yl)cyclohexanol

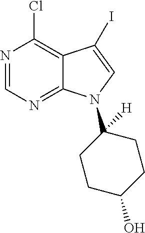

To a solution of trans-4-(4-chloro-7H-pyrrolo[2,3-d]pyrimidin-7-yl)cyclohexanol (Step Z.6, 4.5 g, 17.4 mmol) in DMF (50 mL) was added NIS (3.9 g, 17.4 mmol) and the reaction was stirred at room temperature overnight. 125 mL of water was added to the reaction mixture and the resulting product precipitate was collected by filtration and washed with water. The resulting solid was dried in vacuo to obtain the title compound without further purification. MS m/z 378.0 (M+H+) (Method M).

### Step Z.6: trans-4-(4-chloro-7H-pyrrolo[2,3-d]pyrimidin-7-yl)cyclohexanol

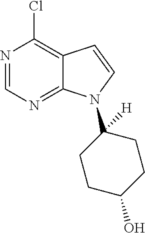

To a mixture of 2-(4,6-dichloropyrimidin-5-yl)acetaldehyde (5.0 g, 26.3 mmol) and trans-4-aminocyclohexanol (3.0 g, 26.3 mmol) in EtOH (66 mL) was added DIEA (5.5 mL, 31.6 mmol). The reaction was heated at 80° C. overnight. The reaction mixture was cooled to room temperature, concentrated in vacuo, and purified with silica gel chromatography (0-10% gradient MeOH in DCM) to obtain the title compound. MS m/z 252.0 (M+H+) (Method M).

### Intermediate AA: 4-((cis-3-(4-amino-5-iodo-7H-pyrrolo[2,3-d]pyrimidin-7-yl)cyclobutyl)methyl)-1-methylpiperazin-2-one

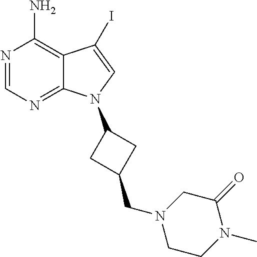

To a stirred solution of [cis-3-(4-amino-5-iodo-pyrrolo[2,3-d]pyrimidin-7-yl)-cyclobutyl]-methanol (Step R.1: 35 mg, 0.1 mmol) and acetonitrile (7.0 mL) was added 2-iodoxybenzoic acid (IBX, 56 mg, 0.2 mmol). The reaction mixture was stirred for 1 hour at 85° C. The reaction mixture was filtered and the filtrate was concentrated in vacuo. To the resulting residue was added DCE (2.0 mL), DIEA (0.15 mL, 1.0 mmol), 1-methylpiperazin-2-one (34 mg, 0.3 mmol) and sodium triacetoxyborohydride (85 mg, 0.4 mmol). The resulting mixture was stirred at room temperature for 1 hour. The reaction mixture was partitioned between Saturated aqueous NaHCO₃ and EtOAc. The combined organic extracts were washed with brine and dried over Na₂SO₄. Concentration in vacuo was followed by purification with silica gel chromatography (0-5% gradient MeOH in DCM) to obtain the title compound. MS m/z 441.1 (M+H+) (Method M).

### Intermediate AB: (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2-oxide

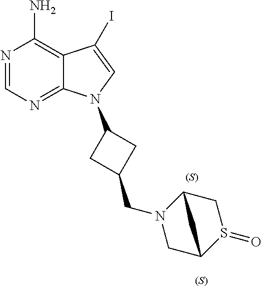

To a suspension of 7-(cis-3-((1S,4S)-2-thia-5-azabicyclo[2.2.1]heptan-5-ylmethyl)cyclobutyl)-5-iodo-7H-pyrrolo[2,3-d]pyrimidin-4-amine (Step AB.1, 46 mg, 0.1 mmol) in MeCN (2 mL) was added oxone solution in water (0.1 M, 1 mL). After stirring at room temperature for 30 min, LCMS (Method M) showed completion of the reaction. The reaction mixture was dried using lypholyzation to give an orange colored solid. The crude product isolated was used for subsequent steps without further purification. MS m/z 458.1 (M+H+) (Method M).

### Step AB.1: 7-(cis-3-((1S,4S)-2-thia-5-azabicyclo[2.2.1]heptan-5-ylmethyl)cyclobutyl)-5-iodo-7H-pyrrolo[2,3-d]pyrimidin-4-amine

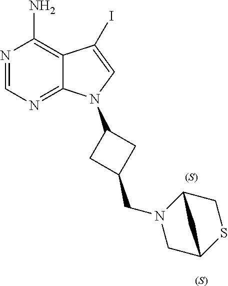

A suspension of [cis-3-(4-amino-5-iodo-pyrrolo[2,3-d]pyrimidin-7-yl)-cyclobutyl]-methanol (Step R.1, 200 mg, 0.58 mmol) and 2-Iodoxybenzoic acid (325 mg, 1.16 mmol) in anhydrous MeCN (10 mL) was stirred at 80° C. After 1 h, LCMS (Method M) showed complete conversion of the primary alcohol in the starting material to the corresponding aldehyde. MS m/z 343.1 (M+H+) (Method M). To the above reaction mixture were added NaBH(OAc)₃ (369 mg, 1.74 mmol), (1S,4S)-2-thia-5-azabicyclo[2.2.1]heptane HCl salt (Prepared from trans-L-Prolinol via the method described in Huang, X. et al. Bioorg. Med. Chem. Lett. 2009, 19, 4130-4133, 124 mg, 0.82 mmol), DIEA (987 uL, 2.59 mmol) and dichloromethane (10 mL). After stirring at room temperature for 30 min, the solvent was evaporated. Water (20 mL) was added. The mixture was extracted with dichloromethane (3×30 mL). The combined dichloromethane extracts were sequentially washed with water (10 mL), brine (10 mL), dried over Na₂SO₄ and evaporated. The resulting residue was purified by flash chromatography (SiO2, 0-10% gradient of MeOH in dichloromethane) to give the title compound as an off-white solid. MS m/z 442.0 (M+H+) (Method M).

### Intermediate AC: (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2,2-dioxide

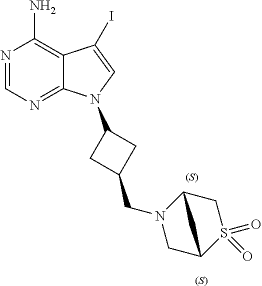

The title compound was prepared in a manner similar to 7-(cis-3-((1S,4S)-2-Thia-5-azabicyclo[2.2.1]heptan-5-ylmethyl)cyclobutyl)-5-iodo-7H-pyrrolo[2,3-d]pyrimidin-4-amine (Step AB.1) starting from cis-3-(4-Amino-5-iodo-pyrrolo[2,3-d]pyrimidin-7-yl)-cyclobutyl]-methanol (Step R.1), and utilizing 2-thia-5-aza-bicyclo[2.2.1]heptane 2,2-dioxide (Prepared from trans-L-Prolinol via the method described in Huang, X. et al. Bioorg. Med. Chem. Lett. 2009, 19, 4130-4133). MS m/z 474.0 (M+H+) (Method M).

### Intermediate AD: 4-((cis-3-(4-amino-5-iodo-7H-pyrrolo[2,3-d]pyrimidin-7-yl)cyclobutyl)methyl)thiomorpholine 1,1-dioxide

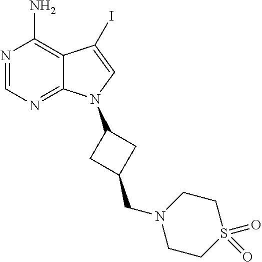

A suspension of [cis-3-(4-amino-5-iodo-pyrrolo[2,3-d]pyrimidin-7-yl)-cyclobutyl]-methanol (Step R.1, 500 mg, 1.45 mmol) and 2-Iodoxybenzoic acid (813 mg, 2.9 mmol) in anhydrous MeCN (20 mL) was stirred at 80° C. for 3 h. LCMS indicated the completion of the reaction. Half of the above reaction mixture was slowly added to a suspension of NaBH(OAc)₃ (0.77 g, 3.63 mmol), thiomorpholine 1,1-dioxide (0.49 g, 3.63 mmol) and DIEA (1.26 mL, 7.28 mmol) in dichloroethane (2 mL). After stirring at room temperature for 2 h, the reaction was quenched with water (20 mL) and extracted with DCM (3×30 mL). The combined DCM layers were washed with brine (20 mL), dried over Na₂SO₄ and evaporated. The resulting residue was purified with silica gel chromatography (0-10% gradient MeOH in DCM) to obtain the title compound as a light brown solid. MS m/z 462.0 (M+H+) (Method M).

### Intermediate AE: N-((cis-3-(4-amino-5-iodo-7H-pyrrolo[2,3-d]pyrimidin-7-yl)cyclobutyl)methyl)acetamide

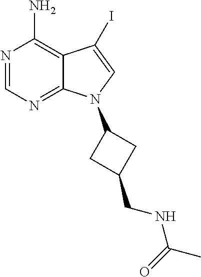

The mixture of 7-(cis-3-aminomethyl-cyclobutyl)-5-iodo-7H-pyrrolo[2,3-d]pyrimidin-4-ylamine (Step AE.1, 274 mg, 0.8 mmol), acetic acid (48 mg, 0.8 mmol), HATU (367 mg, 0.96 mmol) and DIEA (0.17 mL, 0.96 mmol) in 5.0 mL of DMF was stirred at room temperature for 2 h. The reaction mixture was partitioned between EtOAc and brine, the collected organic extracts were dried (Na₂SO₄), concentrated in vacuo, and purified by silica chromatography (EtOAc/Hexanes:1/1) to afford the title compound. MS m/z 386.0 (M+H⁺) (Method M).

### Step AE.1: 7-(cis-3-Aminomethyl-cyclobutyl)-5-iodo-7H-pyrrolo[2,3-d]pyrimidin-4-ylamine

Triphenylphosphine (833 mg, 3.18 mmol) was added to a mixture of 7-(cis-3-azidomethyl-cyclobutyl)-5-iodo-7H-pyrrolo[2,3-d]pyrimidin-4-ylamine (Step AE.2, 920 mg, 2.12 mmol), ammonium hydroxide solution (25%, 1.32 ml, 8.47 mmol), water (1.4 ml), methanol (7 ml) and THF (7 ml). The reaction mixture was stirred overnight at room temperature, then diluted with water, extracted 2× with ethyl acetate, the combined organic phases washed with brine, dried over sodium sulphate and evaporated. Purification of the residue by flash column chromatography, eluting with a gradient of methanol in DCM containing 1% concentrated ammonia solution, gave the title compound as a white solid. ¹H NMR (400 MHz, DMSO) δppm 1.74 (s, br, 2H), 2.06-2.18 (m, 3H), 2.32-2.39 (m, 2H), 2.57-2.60 (m, 2H), 4.95-5.02 (m, 1H), 6.59 (s, br, 2H), 7.68 (s, 1H), 8.08 (s, 1H).

### Step AE.2: 7-(cis-3-azidomethyl-cyclobutyl)-5-iodo-7H-pyrrolo[2,3-d]pyrimidin-4-ylamine

A mixture of toluene-4-sulfonic acid-cis-3-(4-amino-5-iodo-pyrrolo[2,3-d]pyrimidin-7-yl)-cyclobutylmethyl ester (Step AE.3, 18.0 g, 18.1 mmol), sodium azide (4.70 g, 72.2 mmol) and DMF (60 ml) was heated at 65° C. for 1 hour. The cooled reaction mixture was diluted with water, extracted 3× with ethyl acetate, the combined organic phases washed with brine, dried over magnesium sulphate and evaporated to give the title compound as a yellow solid. HPLC/MS t_{R} 0.97 min, M+H 369.9 (Method X).

### Step AE.3: Toluene-4-sulfonic acid-cis-3-(4-amino-5-iodo-pyrrolo[2,3-d]pyrimidin-7-yl)-cyclobutylmethyl ester

para-Toluene sulphonyl chloride (11.52 g, 60.4 mmol) was added portion-wise over 45 minutes to a solution of [cis-3-(4-amino-5-iodo-pyrrolo[2,3-d]pyrimidin-7-yl)-cyclobutyl]-methanol (Step R.1, 7.0 g, 20.14 mmol) in pyridine (20 ml) cooled at −20° C. After 18 hours at −25° C. the reaction mixture was partitioned between 1N sulphuric acid and DCM cooled ar 0° C., extracted 2× with DCM, the combined organic layers dried over sodium sulphate and evaporated to give the title compound as a yellow solid. HPLC/MS t_{R} 1.12 min, M+H 498.9 (Method X).

III Chemical Synthesis compounds according to the invention

### Example 1

### 7-[3-(1,1-Dioxo-1-thiomorpholin-4-ylmethyl)-cyclobutyl]-5-[2-fluoro-3-(tetrahydro-furan-2-ylmethoxy)-phenyl]-7H-pyrrolo[2,3-d]pyrimidin-4-ylamine

A mixture of 2-[2-fluoro-3-(tetrahydro-furan-2-ylmethoxy)-phenyl]-4,4,5,5-tetramethyl-[1,3,2]dioxaborolane (Intermediate A, 175 mg, 0.543 mmol), 5-bromo-7-[3-(1,1-dioxo-1-thiomorpholin-4-ylmethyl)-cyclobutyl]-7H-pyrrolo[2,3-d]pyrimidin-4-ylamine (Intermediate N, 150 mg, 0.362 mmol), K₃PO₄ (154 mg, 0.724 mmol), Na₂CO₃ (77 mg, 0.724 mmol), DMF (20 ml) and water (1 ml) was purged with argon, tetrakis(triphenylphosphine)palladium (21 mg, 0.018 mmol) added, the reaction vessel sealed under argon and heated for 1.5 hours at 100° C. The cooled reaction mixture was diluted with ethyl acetate and washed with water then brine, the organic layers dried over Na₂SO₄ and evaporated. Purification of the residue by normal phase column chromatography, eluting with a gradient of methanol containing 10% concentrated ammonia solution in DCM, gave the title compound as a white solid. ¹H-NMR (400 MHz, DMSO-d₆): δppm 8.13 (s, 1H), 7.64 (s, 1H), 7.24-7.14 (m, 2H), 7.00-6.92 (m, 1H), 6.50 (s, br, 2H), 5.13-5.06 (m, 1H), 4.26-4.20 (m, 1H), 4.14-4.01 (m, 2H), 4.33-4.27 (m, 1H), 4.23-4.17 (m, 1H), 3.11-3.04 (m, 4H), 2.93-2.85 (m, 4H), 2.72-2.66 (m, 2H), 2.55-2.49 (m, 2H), 2.33-2.27 (m, 1H), 2.20-2.14 (m, 2H), 2.05-1.99 (m, 1H), 1.92-1.76 (m, 2H), 1.74-1.68 (m, 1H).

### Example 2

### 7-[3-(1,1-Dioxo-1-thiomorpholin-4-ylmethyl)-cyclobutyl]-5-{2-fluoro-5-[(S)-1-(tetrahydro-furan-2-yl)methoxy]-phenyl}-7H-pyrrolo[2,3-d]pyrimidin-4-ylamine

To the solution of 3-{4-amino-7-[cis-3-(1,1-dioxo-1-thiomorpholin-4-ylmethyl)-cyclobutyl]-7H-pyrrolo[2,3-d]pyrimidin-5-yl}-4-fluoro-phenol (Intermediate O, 91 mg, 0.20 mmol), triphenylphosphine (84 mg, 0.32 mmol) and THF (2.0 ml) was added subsequently (S)-1-(tetrahydro-furan-2-yl)-methanol (29.1 mg, 0.28 mmol) and diisopropyl azodicarboxylate (43 mg, 0.20 mmol) and stirred for 12 days at room temperature. The reaction mixture was partitioned between 1M aqueous NaHCO₃ and ethyl acetate (2×). The organic layers were washed with water and brine, dried over Na₂SO₄ and evaporated. Purification of the residue by normal phase column chromatography, eluting with DCM-MeOH—aqueous ammonia 30% (200:10:1), gave the title compound as a white solid. ¹H-NMR (600 MHz, DMSO-d₆): δppm 8.12 (s, 1H), 7.64 (s, 1H), 7.23 (t, 1H), 6.98-6.93 (m, 2H), 6.06 (bs, 2H), 5.09 (m, 1H), 4.15 (m, 1H), 4.01-3.89 (m, 2H), 3.77 (m, 1H), 3.68 (m, 1H), 3.08 (m, 4H), 2.91 (m, 4H), 2.69 (d, 2H), 2.54 (m, 2H), 2.32 (m, 1H), 2.20 (m, 2H), 1.98 (m, 1H), 1.91-1.80 (m, 2H), 1.66 (m, 1H). HPLC/MS t_{R} 0.75 min, M+H 530.3 (Method X).

### Example 3

### 7-[3-(1,1-Dioxo-1-thiomorpholin-4-ylmethyl)-cyclobutyl]-5-{2-fluoro-5-[(R)-1-(tetrahydro-furan-2-yl)methoxy]-phenyl}-7H-pyrrolo[2,3-d]pyrimidin-4-ylamine

The title compound was prepared in a manner similar to Example 2 using Intermediate O and (R)-1-(tetrahydro-furan-2-yl)-methanol to afford the title compound as white solid: ¹H-NMR (600 MHz, DMSO-d₆): δppm 8.12 (s, 1H), 7.64 (s, 1H), 7.23 (t, 1H), 6.98-6.93 (m, 2H), 6.06 (bs, 2H), 5.09 (m, 1H), 4.15 (m, 1H), 4.01-3.89 (m, 2H), 3.77 (m, 1H), 3.68 (m, 1H), 3.08 (m, 4H), 2.91 (m, 4H), 2.69 (d, 2H), 2.54 (m, 2H), 2.32 (m, 1H), 2.20 (m, 2H), 1.98 (m, 1H), 1.91-1.80 (m, 2H), 1.66 (m, 1H). HPLC/MS t_{R} 0.75 min, M+H 530.3 (Method X).

### Example 4

### 2-(4-Amino-5-{2-fluoro-3-[(S)-1-(tetrahydro-furan-2-yl)methoxy]-phenyl}-pyrrolo[2,3-d]pyrimidin-7-yl)-5-oxa-7-aza-spiro[3.4]octan-6-one

Argon was bubbled through a mixture of 2-(4-amino-5-iodo-pyrrolo[2,3-d]pyrimidin-7-yl)-(E)-5-oxa-7-aza-spiro[3.4]octan-6-one (Intermediate P, 45 mg, 0.117 mmol), 2-{2-fluoro-3-[(S)-1-(tetrahydro-furan-2-yl)methoxy]-phenyl}-4,4,5,5-tetramethyl-[1,3,2]dioxaborolane (Intermediate C, 49 mg, 0.152 mmol), potassium phosphate (50 mg, 0.234 mmol), sodium carbonate (25 mg, 0.234 mmol), DMF (0.8 ml) and water (0.04 ml) at room temperature for 5 minutes. Tetrakistriphenylphosphinepalladium(0) (6.8 mg, 0.006 mmol) was then added, the reaction vessel sealed under argon and heated at 80° C. for 1.5 hours. The cooled reaction mixture was partitioned between water and ethyl acetate, extracted 2× with ethyl acetate, the combined organic layers were washed with brine, dried over sodium sulphate and evaporated. Purification of the residue by reversed phase chromatography (Method R), basification of the product containing fractions with NaHCO₃, extraction with ethyl acetate, drying over Na₂SO₄ and evaporation gave the title compound as a white solid. ¹H NMR (400 MHz, MeOH-d₄) δppm 1.82-1.89 (m, 1H), 1.91-2.14 (m, 3H), 3.46 (td, J=6.4 & 2.9 Hz, 1H) 3.54-3.59 (m, 1H), 3.61 (s, 1H), 3.73 (s, 2H), 3.77-3.86 (m, 1H), 3.86-3.97 (m, 1H) 4.02-4.16 (m, 2H) 4.31 (dd, J=6.3 & 3.9 Hz, 1H), 5.38 (t, J=8.4 Hz, 1H), 6.95-7.05 (m, 1H), 7.18 (q, J=7.9 Hz, 2H), 7.43 (s, 1H), 8.15 (s, 1H).

### Example 5

### 5-{2-Fluoro-3-[(R)-1-(tetrahydro-furan-2-yl)methoxy]-phenyl}-7-[cis-3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate B to give the title compound as white foam: ¹H-NMR (600 MHz, DMSO-d₆): δ ppm 8.16 (s, 1H), 7.64 (s, 1H), 7.18 (m, 2H), 6.96 (m, 1H), 6.01 (bs, 2H), 5.09 (m, 1H), 4.20 (m, 1H), 4.11-4.00 (m, 2H), 3.78 (m, 1H), 3.69 (m, 1H), 2.87 (m, 4H), 2.73-53 (m, 8H), 2.34 (m, 1H), 2.18 (m, 2H), 2.01 (m, 1H), 1.89 (m, 1H), 1.84 (m, 1H), 1.71 (m, 1H). HPLC/MS t_{R} 0.56 min, M+H 514.3 (Method X).

### Example 6

### 5-{2-Fluoro-3-[(S)-1-(tetrahydro-furan-2-yl)methoxy]-phenyl}-7-[cis-3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate C to give the title compound as white foam: ¹H-NMR (600 MHz, DMSO-d₆): δppm 8.16 (s, 1H), 7.64 (s, 1H), 7.18 (m, 2H), 6.96 (m, 1H), 6.01 (bs, 2H), 5.09 (m, 1H), 4.20 (m, 1H), 4.11-4.00 (m, 2H), 3.78 (m, 1H), 3.69 (m, 1H), 2.87 (m, 4H), 2.73-2.53 (m, 8H), 2.34 (m, 1H), 2.18 (m, 2H), 2.01 (m, 1H), 1.89 (m, 1H), 1.84 (m, 1H), 1.71 (m, 1H). HPLC/MS t_{R} 0.56 min, M+H 514.3 (Method X).

### Example 7

### 4-[4-Amino-7-(cis-3-hydroxymethyl-cyclobutyl)-7H-pyrrolo[2,3-d]pyrimidin-5-yl]-2-(7-oxa-bicyclo[2.2.1]hept-1-ylmethoxy)-phenol

In an enzymatic biotransformation, (cis-3-{4-amino-5-[3-(7-oxa-bicyclo[2.2.1]hept-1-ylmethoxy)-phenyl]-pyrrolo[2,3-d]pyrimidin-7-yl}-cyclobutyl)-methanol (Intermediate R, 402 mg, mmol) in acetonitrile (40 ml) and glycerin (50%, 50 ml) in PSE (500 ml) were added to a cell culture solution expressing the human recombinant CYP450 1A1enzyme (2 l) and the mixture incubated at 30° C. for 5.5 hours with oxygenation (200 ml/min) in a 10 litre Wavebag. Afterwards products were extracted from the biotransformation solution by adding XAD-16 resin (125 g) and stirring for 30 minutes. XAD16 was separated by filtration and the products were eluted from the resin with methanol and 2-propanol. Evaporation was followed by reversed phase chromatography to give the title compound as a white solid. ¹H-NMR (600 MHz, DMSO-d₆): δppm 9.01 (s, 1H), 8.09 (s, 1H), 7.40 (s, 1H), 7.06 (d, 1H), 6.89-6.82 (m, 2H), 6.09 (s, br, 2H), 5.10-5.02 (m, 1H), 4.63-4.59 (m, 1H), 4.52-4.47 (m, 1H), 4.29 (s, 2H), 3.49-3.46 (m, 2H), 2.47-2.39 (m, 2H), 2.27-2.22 (m, 2H), 1.75-1.67 (m, 4H), 1.60-1.51 (m, 4H).

### Example 8

### 5-{2-Fluoro-5-[(S)-1-(tetrahydro-furan-2-yl)methoxy]-phenyl}-7-[cis3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate K to give the title compound as a white solid: ¹H-NMR (600 MHz, DMSO-d₆): δppm 8.15 (bs, 1H), 7.63 (s, 1H), 7.23 (t, 1H), 6.95 (m, 2H), 6.05 (bs, 2H), 5.08 (m, 1H), 4.15 (m, 1H), 4.00-3.90 (m, 2H), 3.77 (m, 1H), 3.68 (m, 1H), 2.87 (m, 4H), 2.75-2.53 (m, 8H), 2.34 (m, 1H), 2.19 (m, 2H), 1.98 (m, 1H), 1.87 (m, 1H), 1.83 (m, 1H), 1.67 (m, 1H). HPLC/MS t_{R} 0.59 min, M+H 514.2 (Method X).

### Example 9

### 1-{4-[cis-3-(4-Amino-5-{2-fluoro-3-[(S)-1-(tetrahydro-furan-2-yl)methoxy]-phenyl}-pyrrolo[2,3-d]pyrimidin-7-yl)-cyclobutyl]-piperazin-1-yl}-ethanone

Argon was bubbled through a mixture of 1-{4-[cis-3-(4-amino-5-iodo-pyrrolo[2,3-d]pyrimidin-7-yl)-cyclobutyl]-piperazin-1-yl}-ethanone (Intermediate S, 231 mg, 0.525 mmol), 2-{2-fluoro-3-[(S)-1-(tetrahydro-furan-2-yl)methoxy]-phenyl}-4,4,5,5-tetramethyl-[1,3,2]clioxaborolane (Intermediate C, 211 mg, 0.630 mmol), potassium phosphate (228 mg, 1.05 mmol), sodium carbonate (111 mg, 1.05 mmol), DMF (2.7 ml) and water (0.3 ml) at room temperature for 5 minutes. Tetrakistriphenylphosphinepalladium(0) (30.3 mg, 0.026 mmol) was then added, the reaction vessel sealed under argon and heated at 100° C. for 2.5 hours. The cooled reaction mixture was partitioned between water and DCM, extracted 2× with DCM, the combined organic layers dried over sodium sulphate and evaporated. Purification of the residue by normal phase chromatography, eluting with 4% methanol in DCM, followed by recrystallisation from methanol gave the title compound as an yellow solid. HPLC/MS t_{R} 0.77 min, M+H 509.4 (Method X).

### Example 10

### d₉-1-[4-(cis-3-{4-Amino-5-[2-fluoro-5-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone

Argon was bubbled through a mixture of 1-{4-[cis-3-(4-amino-5-iodo-pyrrolo[2,3-d]pyrimidin-7-yl)-cyclobutyl]-piperazin-1-yl}-ethanone (Intermediate S, 185 mg, 0.420 mmol), d₉-1-[4-fluoro-3-(4,4,5,5-tetramethyl-[1,3,2]clioxaborolan-2-yl)-phenoxymethyl]-7-oxa-bicyclo[2.2.1]heptane (Intermediate M, 195 mg, 0.546 mmol), potassium phosphate (183 mg, 0.840 mmol), sodium carbonate (89 mg, 0.840 mmol), DMF (2.7 ml) and water (0.3 ml) at room temperature for 5 minutes. Tetrakistriphenylphosphinepalladium(0) (24.3 mg, 0.021 mmol) was then added, the reaction vessel sealed under argon and heated at 100° C. for 2.2 hours. The cooled reaction mixture was partitioned between water and DCM, extracted 2× with DCM, the combined organic layers dried over sodium sulphate and evaporated. Purification of the residue by normal phase chromatography, eluting with 5% methanol in DCM gave the title compound as an yellow solid. HPLC/MS t_{R} 0.86 min, M+H 544.5 (Method X). ¹H-NMR (400 MHz, CDCl₃): δppm 8.31 (s, 1H), 7.32 (s, 1H), 7.13 (t, 1H), 7.04-6.98 (m, 1H), 6.96-6.90 (m, 1H), 5.19 (s, br, 2H), 5.14-5.04 (m, 1H), 4.26 (s, 2H), 3.70-3.62 (m, 2H), 3.53-3.45 (m, 2H), 2.84-2.75 (m, 2H), 2.71-2.61 (m, 1H), 2.43-2.24 (m, 6H), 2.09 (s, 3H).

### Example 11

### 5-{2-Fluoro-5-[(S)-1-(tetrahydro-furan-2-yl)methoxy]-phenyl}-7-[cis-3-(1-oxo-1-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate K to give the title compound as a white solid: ¹H-NMR (600 MHz, DMSO-d₆): δppm 8.16 (bs, 1H), 7.50 (s, 1H), 7.23 (t, 1H), 7.00-6.91 (m, 2H), 6.07 (bs, 2H), 4.89 (m, 1H), 4.15 (m, 1H), 4.04-3.89 (m, 2H), 3.78 (m, 1H), 3.68 (m, 1H), 2.90-2.50 (m, 11H), 2.30 (m, 2H), 2.00 (m, 1H), 1.88 (m, 1H), 1.83 (m, 1H), 1.66 (m, 1H). HPLC/MS t_{R} 0.60 min, M+H 500.3 (Method X).

### Example 12

### 5-{2-Fluoro-3-[(S)-1-(tetrahydro-furan-2-yl)methoxy]-phenyl}-7-[cis-3-(1-oxo-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate C to give the title compound as a white solid: ¹H-NMR (600 MHz, DMSO-d₆): δppm 8.17 (bs, 1H), 7.49 (s, 1H), 7.18 (m, 2H), 6.97 (m, 1H), 6.04 (bs, 2H), 4.89 (m, 1H), 4.19 (m, 1H), 4.12-4.00 (m, 2H), 3.78 (m, 1H), 3.69 (m, 1H), 2.90-2.50 (m, 11H), 2.30 (m, 2H), 2.01 (m, 1H), 1.90 (m, 1H), 1.83 (m, 1H), 1.70 (m, 1H). HPLC/MS t_{R} 0.59 min, M+H 500.3 (Method X).

### Example 13

### 5-{2-Fluoro-3-[(R)-1-(tetrahydro-furan-2-yl)methoxy]-phenyl}-7-[cis-3-(1-oxo-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate B to give the title compound as a white solid: ¹H-NMR (600 MHz, DMSO-d₆): δppm 8.17 (bs, 1H), 7.49 (s, 1H), 7.18 (m, 2H), 6.97 (m, 1H), 6.04 (bs, 2H), 4.89 (m, 1H), 4.19 (m, 1H), 4.12-4.00 (m, 2H), 3.78 (m, 1H), 3.69 (m, 1H), 2.90-2.50 (m, 11H), 2.30 (m, 2H), 2.01 (m, 1H), 1.90 (m, 1H), 1.83 (m, 1H), 1.70 (m, 1H). HPLC/MS t_{R} 0.59 min, M+H 500.3 (Method X).

### Example 14

### 2-(4-Amino-5-{2-fluoro-5-[(S)-1-(tetrahydro-furan-2-yl)methoxy]-phenyl}-pyrrolo[2,3-d]pyrimidin-7-yl)-5-oxa-7-aza-spiro[3.4]octan-6-one

Argon was bubbled through a mixture of 2-(4-amino-5-iodo-pyrrolo[2,3-d]pyrimidin-7-yl)-(E)-5-oxa-7-aza-spiro[3.4]octan-6-one (Intermediate P, 95 mg, 0.247 mmol), 2-{2-fluoro-5-[(S)-1-(tetrahydro-furan-2-yl)methoxy]-phenyl}-4,4,5,5-tetramethyl-[1,3,2]dioxaborolane (Intermediate K, 115 mg, 0.357 mmol), potassium phosphate (105 mg, 0.493 mmol), sodium carbonate (52 mg, 0.493 mmol), DMF (2.3 ml) and water (0.12 ml) at room temperature for 5 minutes. Tetrakistriphenylphosphinepalladium(0) (14.3 mg, 0.012 mmol) was then added, the reaction vessel sealed under argon and heated at 100° C. for 3 hours. The cooled reaction mixture was partitioned between water and ethyl acetate, extracted 2× with ethyl acetate, the combined organic layers were washed with brine, dried over sodium sulphate and evaporated. Purification of the residue by normal phase chromatography, eluting with a gradient of methanol in DCM containing 0.5% concentrated ammonium hydroxide solution, gave the title compound as a white solid. ¹H NMR (400 MHz, DMSO-d₆) δ ppm 1.52-1.70 (m, 1H), 1.74 (s, br, 1H), 1.78-1.89 (m, 2H), 1.89-2.09 (m, 1H), 2.79-2.99 (m, 4H), 3.55-3.69 (m, 3H), 3.75 (t, J=7.0 Hz, 1H), 3.81-4.01 (m, 2H), 4.03-4.22 (m, 1H) 5.30 (t, J=8.2 Hz, 1H), 6.87-7.04 (m, 2H), 7.22 (t, J=9.8 Hz, 1H), 7.53 (s, 1H), 7.61 (s, 1H), 8.13 (s, 1H).

### Example 15

### d₉-5-[2-Fluoro-5-(7-oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate M to give the title compound as yellow foam: ¹H-NMR (600 MHz, DMSO-d₆): δppm 8.15 (bs, 1H), 7.51 (s, 1H), 7.23 (m, 1H), 7.06-6.96 (m, 2H), 6.07 (bs, 2H), 4.89 (m, 1H), 4.27 (s, 2H), 2.86 (m, 2H), 2.76 (m, 5H), 2.64 (m, 4H), 2.30 (m, 2H). HPLC/MS t_{R} 0.66 min, M=534.3 (Method X).

### Example 16

### d₉-5-[2-Fluoro-5-(7-oxa-bicyclo[2.2.1]hept-1-ylmethoxy)-phenyl]-7-[cis-3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate M to give the title compound as beige foam: ¹H-NMR (600 MHz, DMSO-d₆): δ ppm 8.12 (s, 1H), 7.64 (s, 1H), 7.24 (t, 1H), 7.04-6.98 (m, 2H), 6.06 (bs, 2H), 5.09 (m, 1H), 4.27 (s, 2H), 2.87 (m, 4H), 2.74-2.52 (m, 8H), 2.35 (m, 1H), 2.19 (m, 2H). HPLC/MS t_{R} 0.63 min, M=549.3 (Method X).

### Example 17

### 5-{2-Fluoro-5-[(R)-1-(tetrahydro-furan-2-yl)methoxyl]-phenyl}-7-[cis-3-(1-oxo-1-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate J to give the title compound as a white solid: ¹H-NMR (600 MHz, DMSO-d₆): δppm 8.12 (s, 1H), 7.63 (s, 1H), 7.23 (t, 1H), 6.95 (m, 2H), 6.05 (bs, 2H), 5.09 (m, 1H), 4.15 (m, 1H), 4.00-3.90 (m, 2H), 3.77 (m, 1H), 3.68 (m, 1H), 2.87 (m, 4H), 2.75-2.53 (m, 8H), 2.35 (m, 1H), 2.19 (m, 2H), 1.98 (m, 1H), 1.87 (m, 1H), 1.83 (m, 1H), 1.67 (m, 1H). HPLC/MS t_{R} 0.58 min, M+H 514.3 (Method X).

### Example 18

### d₉-5-[2-Fluoro-3-(7-oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate E to give the title compound as yellow foam: ¹H-NMR (600 MHz, DMSO-d₆): δppm 8.15 (bs, 1H), 7.50 (s, 1H), 7.28-7.18 (m, 2H), 6.98 (m, 1H), 6.06 (bs, 2H), 4.90 (m, 1H), 4.38 (s, 2H), 2.86 (m, 2H), 2.76 (m, 5H), 2.64 (m, 4H), 2.32 (m, 2H). HPLC/MS t_{R} 0.66 min, M=534.4 (Method X).

### Example 19

### d₉-5-[2-Fluoro-3-(7-oxa-bicyclo[2.2.1]hept-1-ylmethoxy)-phenyl]-7-[cis-3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate E to give the title compound as beige foam: ¹H-NMR (600 MHz, DMSO-d₆): δ ppm 8.14 (bs, 1H), 7.65 (s, 1H), 7.28-7.19 (m, 2H), 6.97 (m, 1H), 6.05 (bs, 2H), 5.09 (m, 1H), 4.38 (s, 2H), 2.92-2.81 (m, 4H), 2.73-2.52 (m, 8H), 2.34 (m, 1H), 2.18 (m, 2H). HPLC/MS t_{R} 0.64 min, M=548.4 (Method X).

### Example 20

### 5-{2-Fluoro-5-[(R)-1-(tetrahydro-furan-2-yl)methoxyl]-phenyl}-7-[cis-3-(1-oxo-1-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate J to give the title compound as a white solid: ¹H-NMR (600 MHz, DMSO-d₆): δ ppm 8.13 (s, 1H), 7.51 (s, 1H), 7.23 (t, 1H), 7.02-6.90 (m, 2H), 6.08 (bs, 2H), 4.90 (m, 1H), 4.15 (m, 1H), 4.02-3.90 (m, 2H), 3.77 (m, 1H), 3.68 (m, 1H), 2.86 (m, 2H), 2.76 (m, 5H), 2.64 (m, 4H), 2.32 (m, 2H), 2.00 (m, 1H), 1.87 (m, 1H), 1.83 (m, 1H), 1.66 (m, 1H). HPLC/MS t_{R} 0.60 min, M+H 500.3 (Method X).

### Example 21

### 2-(4-Amino-5-{2-fluoro-5-[(S)-1-(tetrahydro-furan-2-yl)methoxy]-phenyl}-pyrrolo[2,3-d]pyrimidin-7-yl)-5-oxa-7-aza-spiro[3.4]octan-6-one

The title compound was prepared in an analagous fashion to Example 14 by substituting 2-{2-fluoro-5-[(S)-1-(tetrahydro-furan-2-yl)methoxy]-phenyl}-4,4,5,5-tetramethyl-[1,3,2]dioxaborolane (Intermediate K) with 2-{2-fluoro-5-[(R)-1-(tetrahydro-furan-2-yl)methoxy]-phenyl}-4,4,5,5-tetramethyl-[1,3,2]dioxaborolane (Intermediate J). The title compound was obtained as a pink solid. HPLC/MS t_{R} 0.71 min, M+H 454.3 (Method X).

### Example 22

### d₉-1-[4-(cis-3-{4-Amino-5-[2-fluoro-3-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone

The title compound was prepared in an analagous fashion to Example 10 by substituting d₉-1-[4-fluoro-3-(4,4,5,5-tetramethyl-[1,3,2]dioxaborolan-2-yl)-phenoxymethyl]-7-oxa-bicyclo[2.2.1]heptane (Intermediate M) with d₉-1-[2-fluoro-3-(4,4,5,5-tetramethyl-[1,3,2]dioxaborolan-2-yl)-phenoxymethyl]-7-oxa-bicyclo[2.2.1]heptane (Intermediate E).

The title compound was obtained as a beige solid. ¹H-NMR (400 MHz, DMSO-d₆): δppm 8.13 (s, 1H), 7.52 (s, 1H), 7.27-7.23 (m, 1H), 7.23-7.17 (m, 1H), 7.00-6.95 (m, 1H), 6.02 (s, br, 1H), 4.98-4.90 (m, 1H), 4.40 (s, 2H), 3.47-3.38 (m, 4H), 2.70-2.58 (m, 3H), 2.40-2.22 (m, 6H), 1.99 (s, 3H).

### Example 23

### 5-[2-Fluoro-3-(oxetan-2-ylmethoxy)-phenyl]-7-[cis-3-(1-oxo-thiomorpholin-4-yl)-cyclobutyl]-7H-pyrrolo[2,3-d]pyrimidin-4-ylamine

A mixture of 2-[2-fluoro-3-(oxetan-2-ylmethoxy)-phenyl]-4,4,5,5-tetramethyl-[1,3,2]dioxaborolane (Intermediate F, 181 mg, 0.469 mmol), 5-iodo-7-[cis-3-(1-oxo-thiomorpholin-4-yl)-cyclobutyl]-7H-pyrrolo[2,3-d]pyrimidin-4-ylamine (Intermediate U, 110 mg, 0.235 mmol), K₃PO₄ (102 mg, 0.469 mmol), Na₂CO₃ (50 mg, 0.469 mmol), DMF (1.8 ml) and water (0.2 ml) was purged with argon, tetrakis(triphenylphosphine)palladium (14 mg, 0.012 mmol) added, the reaction vessel sealed under argon and heated for 2.2 hours at 100° C. The cooled reaction mixture was diluted with DCM and washed with water, the organic layers dried over Na₂SO₄ and evaporated. Purification of the residue by normal phase column chromatography, eluting with a gradient of methanol in DCM, gave the title compound as a beige solid. HPLC/MS t_{R} 0.64 min, M+H 486.2 (Method X). ¹H-NMR (400 MHz, CDCl₃): δ ppm 8.31 (s, 1H), 7.32 (s, 1H), 7.20-7.12 (m, 1H), 7.10-6.98 (m, 2H), 5.22-5.05 (m, 4H), 4.77-4.63 (m, 2H), 4.30-4.20 (m, 2H), 3.05-2.61 (m, 11H), 2.38-2.24 (m, 2H).

### Example 24

### 5-[2-Fluoro-3-(oxetan-2-ylmethoxy)-phenyl]-7-[cis-3-(1-oxo-thiomorpholin-4-ylmethyl)-cyclobutyl]-7H-pyrrolo[2,3-d]pyrimidin-4-ylamine

The title compound was prepared in an analagous fashion to Example 23 by substituting 5-iodo-7-[cis-3-(1-oxo-thiomorpholin-4-yl)-cyclobutyl]-7H-pyrrolo[2,3-d]pyrimidin-4-ylamine (Intermediate U) with 5-Iodo-7-[cis-3-(1-oxo-thiomorpholin-4-ylmethyl)-cyclobutyl]-7H-pyrrolo[2,3-d]pyrimidin-4-ylamine (Intermediate Q). The title compound was obtained as a pale-yellow glass. HPLC/MS t_{R} 0.63 min, M+H 500.2 (Method X). ¹H-NMR (400 MHz, CDCl₃): δ ppm 8.32 (s, 1H), 7.21 (s, 1H), 7.18-7.11 (m, 1H), 7.09-6.95 (m, 2H), 5.22-5.06 (m, 4H), 4.75-4.64 (m, 2H), 4.25-4.20 (m, 2H), 3.14-3.04 (m, 2H), 2.92-2.63 (m, 8H), 2.62 (d, 2H), 2.44-2.36 (m, 1H), 2.21-2.10 (m, 2H).

### Example 25

### 1-{4-[cis-3-(4-Amino-5-{2-fluoro-5-[(R)-1-(tetrahydro-furan-2-yl)methoxy]-phenyl}-pyrrolo[2,3-d]pyrimidin-7-yl)-cyclobutyl]-piperazin-1-yl}-ethanone

The title compound was prepared in an analagous fashion to Example 10 by substituting d₉-1-[4-fluoro-3-(4,4,5,5-tetramethyl-[1,3,2]dioxaborolan-2-yl)-phenoxymethyl]-7-oxa-bicyclo[2.2.1]heptane (Intermediate M) with 2-{2-fluoro-5-[(R)-1-(tetrahydro-furan-2-yl)methoxy]-phenyl}-4,4,5,5-tetramethyl-[1,3,2]dioxaborolane (Intermediate J). The title compound was obtained as a white solid. ¹H-NMR (400 MHz, DMSO-d₆): δ ppm 1.59-1.71 (m, 1H), 1.74-1.92 (m, 1H), 1.92-2.05 (m, 4H), 2.18-2.39 (m, 6H), 2.55-2.69 (m, 3H), 3.36-3.50 (m, 4H), 3.61-3.80 (m, 2H), 3.88-4.01 (m, 2H), 4.08-4.19 (m, 1H), 4.84-4.96 (m, 1H), 5.90-6.22 (m, 2H), 6.89-7.00 (m, 2H), 7.21 (t, 1H), 7.50 (s, 1H), 8.12 (s, 1H).

### Example 26

### 3-(4-Amino-5-{2-fluoro-5-[(S)-1-(tetrahydro-furan-2-yl)methoxy]-phenyl}-pyrrolo[2,3-d]pyrimidin-7-yl)-1-hydroxymethyl-cyclobutanol

A mixture of 2-{2-fluoro-5-[(S)-1-(tetrahydro-furan-2-yl)methoxy]-phenyl}-4,4,5,5-tetramethyl-[1,3,2]dioxaborolane (Intermediate K, 280 mg, 0.868 mmol), 3-(4-amino-5-iodo-pyrrolo[2,3-d]pyrimidin-7-yl)-1-hydroxymethyl-cyclobutanol (Intermediate V, 250 mg, 0.694 mmol), K₃PO₄ (302 mg, 1.39 mmol), Na₂CO₃ (147 mg, 1.39 mmol), DMF (2.7 ml) and water (0.3 ml) was purged with argon, tetrakis(triphenylphosphine)palladium (40 mg, 0.035 mmol) added, the reaction vessel sealed under argon and heated for 2 hours at 100° C. The cooled reaction mixture was diluted with DCM and washed with water, the organic layers dried over Na₂SO₄ and evaporated. Purification of the residue by normal phase column chromatography, eluting with a gradient of methanol in DCM, gave the title compound as a colourless glass. HPLC/MS t_{R} 0.75 min, M+H 429.3 (Method X).

### Example 27 and 28

### d₉-(Z)-3-{4-Amino-5-[2-fluoro-3-(7-oxa-bicyclo[2.2.1]hept-1-ylmethoxy)-phenyl]-pyrrolo[2,3-d]pyrimidin-7-yl}-1-hydroxymethyl-cyclobutanol and d₂-(E)-3-{4-Amino-5-[2-fluoro-3-(7-oxa-bicyclo[2.2.1]hept-1-ylmethoxy)-phenyl]-pyrrolo[2,3-d]pyrimidin-7-yl}-1-hydroxymethyl-cyclobutanol

The title compounds were prepared in an analagous fashion to Example 26 by substituting 2-{2-fluoro-5-[(S)-1-(tetrahydro-furan-2-yl)methoxy]-phenyl}-4,4,5,5-tetramethyl-[1,3,2]dioxaborolane (Intermediate K) with d₉-1-[2-fluoro-3-(4,4,5,5-tetramethyl-[1,3,2]dioxaborolan-2-yl)-phenoxymethyl]-7-oxa-bicyclo[2.2.1]heptane (Intermediate E). The isolated solid was purified by normal phase chromatography, eluting with a gradient of methanol in DCM, to give the title compounds.

First eluting (Z)-isomer: yellow solid. HPLC/MS t_{R} 0.78 min, M+H 464.3 (Method X). ¹H NMR (400 MHz, DMSO-d₆) δ ppm 2.33-2.43 (m, 2H), 2.65-2.76 (m, 2H), 3.38 (d, 2H), 4.37 (s, 2H), 4.80-4.92 (m, 2H), 5.26 (s, 1H), 6.93-7.01 (m, 1H), 7.14-7.29 (m, 2H), 7.65 (s, 1H), 8.11 (s, 1H).

Second eluting (E)-isomer: yellow solid. HPLC/MS t_{R} 0.78 min, M+H 464.3 (Method X). ¹H NMR (400 MHz, DMSO-d₆) δ ppm 2.22-2.36 (m, 2H), 2.55-2.72 (m, 2H), 3.11-3.29 (m, 2H), 4.36 (s, 2H), 4.87 (t, 1H), 5.09 (s, 1H), 5.40 (quin, 1H), 6.89-7.01 (m, 1H), 7.13-7.28 (m, 2H), 7.49 (s, 1H), 8.12 (s, 1H).

### Example 29

### (E)-3-(4-Amino-5-{2-fluoro-3-[(S)-1-(tetrahydro-furan-2-yl)methoxy]-phenyl}-pyrrolo[2,3-d]pyrimidin-7-yl)-1-hydroxymethyl-cyclobutanol

The title compounds were prepared in an analagous fashion to Example 26 by substituting 2-{2-fluoro-5-[(S)-1-(tetrahydro-furan-2-yl)methoxy]-phenyl}-4,4,5,5-tetramethyl-[1,3,2]dioxaborolane (Intermediate K) with 2-{2-fluoro-3-[(S)-1-(tetrahydro-furan-2-yl)methoxy]-phenyl}-4,4,5,5-tetramethyl-[1,3,2]dioxaborolane (Intermediate C). The isolated solid was purified by normal phase chromatography, eluting with a gradient of methanol in DCM, to give the title compound as a beige solid. ¹H NMR (400 MHz, CDCl₃) δ ppm 1.76-2.16 (m, 4H), 2.57-2.70 (m, 2H), 2.82-2.95 (m, 2H), 3.72 (s, 2H), 3.79-3.89 (m, 1H), 3.89-3.99 (m, 1H), 4.09 (d, J=5.1 Hz, 2H), 4.26-4.37 (m, 1H), 5.19 (s, br, 2H), 5.41 (quin, J=8.5 Hz, 1H), 6.91-7.05 (m, 2H), 7.07-7.17 (m, 2H), 8.29 (s, 1H).

### Example 30

### 5-[2-Fluoro-5-(7-oxa-bicyclo[2.2.1]hept-1-ylmethoxy)-phenyl]-7-(cis-3-thiomorpholin-4-yl-cyclobutyl)-7H-pyrrolo[2,3-d]pyrimidin-4-ylamine

The title compounds were prepared in an analagous fashion to Example 10 by substituting 1-{4-[3-(4-amino-5-iodo-pyrrolo[2,3-d]pyrimidin-7-yl)-cyclobutyl]-piperazin-1-yl}-ethanone (Intermediate S) with 5-iodo-7-(3-thiomorpholin-4-yl-cyclobutyl)-7H-pyrrolo[2,3-d]pyrimidin-4-ylamine (Intermediate W). Following chromatographic purification, recrystallisation from methanol gave the title compound as a white solid. ¹H NMR (400 MHz, DMSO-d₆) δ ppm 8.12 (s, 1H), 7.50 (s, 1H), 7.26-7.18 (m, 1H), 7.05-6.94 (m, 2H), 6.05 (s, br, 1H), 5.94-5.83 (m, 1H), 4.24 (s, 2H), 2.71-2.41 (m, 11H), 2.31-2.18 (m, 2H).

### Example 31

### 5-[2-Fluoro-3-(7-oxa-bicyclo[2.2.1]hept-1-ylmethoxy)-phenyl]-7-[cis-3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate D to give the title compound as beige foam: ¹H-NMR (600 MHz, DMSO-d₆): δ ppm 8.12 (s, 1H), 7.65 (s, 1H), 7.28-7.17 (m, 2H), 6.98 (m, 1H), 6.05 (bs, 2H), 5.10 (m, 1H), 4.52 (m, 1H), 4.38 (s, 2H), 2.90-2.82 (m, 4H), 2.73-2.52 (m, 8H), 2.34 (m, 1H), 2.18 (m, 2H), 1.73-1.66 (m, 4H), 1.61-1.55 (m, 4H). HPLC/MS t_{R} 0.63 min, M+H=540.2 (Method X).

### Example 32

### 5-[2-Fluoro-3-(7-oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate D to give the title compound as beige foam: ¹H-NMR (600 MHz, DMSO-d₆): δ ppm 8.13 (s, 1H), 7.50 (s, 1H), 7.27-7.18 (m, 2H), 6.98 (m, 1H), 6.06 (bs, 2H), 4.90 (m, 1H), 4.52 (m, 1H), 4.38 (s, 2H), 2.86 (m, 2H), 2.76 (m, 5H), 2.64 (m, 4H), 2.30 (m, 2H), 1.73-1.66 (m, 4H), 1.61-1.55 (m, 4H). HPLC/MS t_{R} 0.66 min, M+H=526.2 (Method X).

### Example 33

### 5-[2-Fluoro-5-(7-oxa-bicyclo[2.2.1]hept-1-ylmethoxy)-phenyl]-7-[cis-3-(1-oxo-thiomorpholin-4-ylmethyl)-cyclobutyl]-7H-pyrrolo[2,3-d]pyrimidin-4-ylamine

The title compound was prepared in a manner similar to Example 1 via coupling Intermediate Q with Intermediate L to give the title compound as beige foam: ¹H-NMR (600 MHz, DMSO-d₆): δ ppm 8.12 (s, 1H), 7.64 (s, 1H), 7.24 (s, 1H), 7.04-6.99 (m, 2H), 6.07 (bs, 2H), 5.09 (m, 1H), 4.50 (m, 1H), 4.28 (s, 2H), 2.90-2.82 (m, 4H), 2.73-2.52 (m, 8H), 2.35 (m, 1H), 2.19 (m, 2H), 1.74-1.66 (m, 4H), 1.61-1.53 (m, 4H). HPLC/MS t_{R} 0.64 min, M+H=540.2 (Method X).

### Example 34

### 5-[2-Fluoro-5-(7-oxa-bicyclo[2.2.1]hept-1-ylmethoxy)-phenyl]-7-[cis-3-(1-oxo-thiomorpholin-4-yl)-cyclobutyl]-7H-pyrrolo[2,3-d]pyrimidin-4-ylamine

The title compound was prepared in a manner similar to Example 1 via coupling Intermediate U with Intermediate L to give the title compound as white foam: ¹H-NMR (600 MHz, DMSO-d₆): δ ppm 8.13 (s, 1H), 7.51 (s, 1H), 7.23 (t, 1H), 7.06-6.95 (m, 2H), 6.09 (bs, 2H), 4.90 (m, 1H), 4.50 (m, 1H), 4.28 (s, 2H), 2.92-2.35 (m, 11H), 2.32 (m, 2H), 1.70-1.53 (m, 8H). HPLC/MS t_{R} 0.66 min, M+H=526.3 (Method X).

### Example 35

### 1-{4-[cis-3-(4-Amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-ethanone

Argon was bubbled through a mixture of 1-{4-[cis-3-(4-amino-5-iodo-pyrrolo[2,3-d]pyrimidin-7-yl)-cyclobutyl]-piperazin-1-yl}-ethanone (Intermediate T, 566 mg, 1.29 mmol), 2-{2-Fluoro-5-[(S)-1-(tetrahydro-furan-2-yl)methoxy]-phenyl}-4,4,5,5-tetramethyl-[1,3,2]dioxaborolane (Intermediate K, 538 mg, 1.67 mmol), potassium phosphate (559 mg, 2.57 mmol), sodium carbonate (273 mg, 2.57 mmol), DMF (5.4 ml) and water (0.6 ml) at room temperature for 5 minutes. Tetrakistriphenylphosphinepalladium(0) (74.3 mg, 0.064 mmol) was then added, the reaction vessel sealed under argon and heated at 100° C. for 4.2 hours. The cooled reaction mixture was partitioned between water and DCM, extracted 2× with DCM, the combined organic layers dried over sodium sulphate and evaporated. Purification of the residue by normal phase chromatography, eluting with 10% methanol in DCM, followed by recrystallisation of the product containing fractions from a 30:1 mixture of methanol/water gave the title compound as a white solid. HPLC/MS t_{R} 0.69 min, M+H 509.3 (Method X). ¹H-NMR (400 MHz, DMSO-d₆): δ ppm 8.11 (s, 1H), 7.50 (s, 1H), 7.21 (t, 1H), 6.99-6.88 (m, 2H), 6.04 (s, br, 2H), 5.95-5.83 (m, 1H), 4.17-4.09 (m, 1H), 4.00-3.89 (m, 2H), 3.75 (q, 1H), 3.66 (q, 1H), 3.46-3.38 (m, 4H), 2.68-2.51 (m, 4H), 2.38-2.23 (m, 5H), 2.03-1.91 (m, 4H), 1.91-1.75 (m, 2H), 1.71-1.59 (m, 1H).

### Example 36

### 1-{4-[trans-3-(4-Amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-ethanone

Argon was bubbled through a mixture of 1-{4-[3-(4-amino-5-iodo-pyrrolo[2,3-d]pyrimidin-7-yl)-cyclobutyl]-piperazin-1-yl}-ethanone (Intermediate S, 566 mg, 1.29 mmol), 2-{2-Fluoro-5-[(S)-1-(tetrahydro-furan-2-yl)methoxy]-phenyl}-4,4,5,5-tetramethyl-[1,3,2]dioxaborolane (Intermediate K, 538 mg, 1.67 mmol), potassium phosphate (559 mg, 2.57 mmol), sodium carbonate (273 mg, 2.57 mmol), DMF (5.4 ml) and water (0.6 ml) at room temperature for 5 minutes. Tetrakistriphenylphosphinepalladium(0) (74.3 mg, 0.064 mmol) was then added, the reaction vessel sealed under argon and heated at 100° C. for 4.2 hours. The cooled reaction mixture was partitioned between water and DCM, extracted 2× with DCM, the combined organic layers dried over sodium sulphate and evaporated. Purification of the residue by normal phase chromatography, eluting with 10% methanol in DCM, gave the compound of Example 35 followed by the product containing fractions. Recrystallisation of the product containing fractions from methanol gave the title compound as a white solid. HPLC/MS t_{R} 0.69 min, M+H 509.3 (Method X). ¹H-NMR (400 MHz, DMSO-d₆): δ ppm 8.11 (s, 1H), 7.61 (s, 1H), 7.21 (t, 1H), 6.98-6.89 (m, 2H), 6.03 (s, br, 2H), 5.31-5.19 (m, 1H), 4.18-4.10 (m, 1H), 4.00-3.89 (m, 2H), 3.75 (q, 1H), 3.66 (q, 1H), 3.48-3.41 (m, 4H), 2.99-2.91 (s, 1H), 2.60-2.44 (m, 4H), 2.38-2.23 (m, 4H), 2.04-1.92 (m, 4H), 1.92-1.75 (m, 2H), 1.71-1.60 (m, 1H).

### Example 37

### 5-{2-Fluoro-5-[(S)-1-(tetrahydro-furan-2-yl)methoxy]-phenyl}-7-(cis-3-piperazin-1-yl-cyclobutyl)-7H-pyrrolo[2,3-d]pyrimidin-4-ylamine

A mixture of 1-{4-[cis-3-(4-amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-ethanone (Example 35, 100 mg, 0.197 mmol), 4M hydrochloric acid (1 ml) and methanol (1 ml) was heated for 16 hours at 50° C. The cooled reaction mixture was neutralised with aqueous NaHCO₃ solution, extracted with DCM, the combined organic extracts dried over Na₂SO₄ and evaporated to give the title compound as a white solid. HPLC/MS t_{R} 0.67 min, M+H 467.3 (Method X).

### Example 38

### 5-{2-Fluoro-5-[(S)-1-(tetrahydro-furan-2-yl)methoxy]-phenyl}-7-(trans-3-piperazin-1-yl-cyclobutyl)-7H-pyrrolo[2,3-d]pyrimidin-4-ylamine

The title compounds were prepared in an analagous fashion to Example 37 by substituting 1-{4-[cis-3-(4-amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-ethanone (Example 35) with 1-{4-[trans-3-(4-amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-ethanone (Example 36). The title compound was obtained as a white solid. HPLC/MS t_{R} 0.66 min, M+H 467.2 (Method X).

### Example 39

### 4-[cis-3-(4-Amino-5-{2-fluoro-5-[(S)-1-(tetrahydro-furan-2-yl)methoxy]-phenyl}-pyrrolo[2,3-d]pyrimidin-7-yl)-cyclobutyl]-piperazine-1-carboxylic acid methyl ester

Methyl chloroformate (3.16 mg, 0.030 mmol) was added to a mixture of 5-{2-fluoro-5-[(S)-1-(tetrahydro-furan-2-yl)methoxy]-phenyl}-7-(cis-3-piperazin-1-yl-cyclobutyl)-7H-pyrrolo[2,3-d]pyrimidin-4-ylamine (Example 37, 13 mg, 0.025 mmol), triethylamine (5.08 mg, 0.050 mmol) and DCM (1 ml) at room temperature. After standing at room temperature for 14 hours the reaction mixture was partitioned between aqueous NaHCO₃ and DCM, the DCM layers dried over Na₂SO₄ and evaporated. Purification of the residue by reversed phase chromatography (Method R), elution of the product containing fractions through a Bond Elut SCX cartridge, releasing with a solution of ammonia in methanol (7M) and evaporation gave the title compound as a white solid. HPLC/MS t_{R} 0.78 min, M+H 525.3 (Method X). ¹H-NMR (400 MHz, CDCl₃): δ ppm 8.31 (s, 1H), 7.34 (s, br, 1H), 7.10 (t, 1H), 6.97-6.84 (m, 2H), 5.20-5.04 (m, 3H), 4.31-4.22 (m, 1H), 4.01-3.78 (m, 4H), 3.56-3.46 (m, 4H), 2.85-2.75 (m, 2H), 2.73-2.62 (m, 1H), 2.43-2.27 (m, 6H), 2.12-2.90 (m, 3H), 1.80-1.69 (m, 1H).

### Example 40

### 1-{4-[cis-3-(4-Amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-2-hydroxy-ethanone

Acetoxyacetyl chloride (5.27 mg, 0.039 mmol) was added to a mixture of 5-{2-fluoro-5-[(S)-1-(tetrahydro-furan-2-yl)methoxy]-phenyl}-7-(cis-3-piperazin-1-yl-cyclobutyl)-7H-pyrrolo[2,3-d]pyrimidin-4-ylamine (Example 37, 15 mg, 0.032 mmol), triethylamine (6.51 mg, 0.064 mmol) and DCM (1 ml) at room temperature. After standing at room temperature for 15 hours the reaction mixture was partitioned between aqueous NaHCO₃ and DCM, the DCM layers dried over Na₂SO₄ and evaporated. The residue was then taken up in methanol (2 ml), K₂CO₃ (10 mg) added. After stirring for a further 20 hours at room temperature, the reaction mixture was partitioned between aqueous NaHCO₃ and DCM, the DCM layers dried over Na₂SO₄ and evaporated. Purification of the residue by reversed phase chromatography (Method R), elution of the product containing fractions through a Bond Elut SCX cartridge, releasing with a solution of ammonia in methanol (7M) and evaporation gave the title compound as a white solid. HPLC/MS t_{R} 0.86 min, M+H 525.3 (Method X). ¹H-NMR (400 MHz, CDCl₃): δ ppm 8.28 (s, 1H), 7.36 (s, 1H), 7.13 (t, 1H), 6.97-6.86 (m, 2H), 5.80 (s, br, 2H), 5.16-5.05 (m, 1H), 4.32-4.22 (m, 1H), 4.17 (s, 2H), 4.03-3.80 (m, 4H), 3.78-3.69 (m, 2H), 3.38-3.29 (m, 2H), 2.89-2.67 (m, 2H), 2.66-2.65 (m, 1H), 2.50-2.30 (m, 6H), 2.14-1.89 (m, 2H), 1.84-1.69 (m, 1H).

### Example 41

### (S)-1-{4-[cis-3-(4-Amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-2-hydroxy-propan-1-one

The title compounds were prepared in an analagous fashion to Example 40 by substituting acetoxyacetyl chloride with (S)-2-acetoxypropionyl chloride. The title compound was obtained as a white solid. HPLC/MS t_{R} 0.86 min, M+H 539.3 (Method X). ¹H-NMR (400 MHz, CDCl₃): δ ppm 8.30 (s, 1H), 7.37 (s, 1H), 7.11 (t, 1H), 6.97-6.86 (m, 2H), 5.58 (s, br, 2H), 5.15-5.04 (m, 1H), 4.52-4.40 (m, 1H), 4.31-4.22 (m, 1H), 4.03-3.60 (m, 6H), 3.51-3.40 (m, 2H), 2.89-2.76 (m, 2H), 2.76-2.63 (m, 1H), 2.49-2.29 (m, 6H), 2.13-1.87 (m, 3H), 1.81-1.69 (m, 1H), 1.35 (d, 3H).

### Example 42

### d₉-5-[2-Fluoro-5-(7-oxa-bicyclo[2.2.1]hept-1-ylmethoxy)-phenyl]-7-(cis-3-piperazin-1-yl-cyclobutyl)-7H-pyrrolo[2,3-d]pyrimidin-4-ylamine

The title compounds were prepared in an analagous fashion to Example 37 by substituting 1-{4-[cis-3-(4-amino-5-{2-fluoro-5-[(S)-1-(tetrahydro-furan-2-yl)methoxy]-phenyl}-pyrrolo[2,3-d]pyrimidin-7-yl)-cyclobutyl]-piperazin-1-yl}-ethanone (Example 35) with d₉-1-[4-(cis-3-{4-amino-5-[2-fluoro-5-(7-oxa-bicyclo[2.2.1]hept-1-ylmethoxy)-phenyl]-pyrrolo[2,3-d]pyrimidin-7-yl}-cyclobutyl)-piperazin-1-yl]-ethanone (Example 10). The title compound was obtained as a brown glass. HPLC/MS t_{R} 0.77 min, M+H 502.6 (Method X). ¹H-NMR (400 MHz, CDCl₃): δ ppm 8.31 (s, 1H), 7.32 (s, 1H), 7.10 (t, 1H), 7.01-6.95 (m, 1H), 6.95-6.88 (m 1H), 5.16-5.00 (m, 3H), 4.24 (s, 2H), 2.99-2.85 (m, 4H), 2.82-2.71 (m, 2H), 2.69-2.58 (m, 1H), 2.49-2.22 (m, 4H), 2.22-2.06 (m, 2H).

### Example 43

### (±)-7-[cis-3-(1,1-Dioxo-1-thiomorpholin-4-ylmethyl)-cyclobutyl]-5-[2-fluoro-5-(tetrahydro-furan-2-ylmethoxy)-phenyl]-7H-pyrrolo[2,3-d]pyrimidin-4-ylamine

A mixture of (±)-2-[2-fluoro-5-(tetrahydro-furan-2-ylmethoxy)-phenyl]-4,4,5,5-tetramethyl-[1,3,2]dioxaborolane (Intermediate I, 35 mg, 0.109 mmol), 5-bromo-7-[3-(1,1-dioxo-1-thiomorpholin-4-ylmethyl)-cyclobutyl]-7H-pyrrolo[2,3-d]pyrimidin-4-ylamine (Intermediate N, 30 mg, 0.072 mmol), and Na₂CO₃ (15.4 mg, 0.144 mmol) in THF (2.0 ml) and water (0.5 ml) was purged with nitrogen. Tetrakis(triphenylphosphine)palladium (4.2 mg, 0.004 mmol) was added and the reaction vessel was sealed under nitrogen and heated under microwave irradiation for 10 minutes at 120° C. The cooled reaction mixture was diluted with ethyl acetate and sequentially washed with water and brine. The organic layers were dried over Na₂SO₄ and evaporated. The resulting residue was purified by reverse phase preparative HPLC (Method S), yielding the title compound as a white solid. MS m/z 530.2 (M+H⁺) (Method M). ¹H-NMR (400 MHz, MeOD-d₄): δ ppm 8.04 (s, 1H), 7.38 (s, 1H), 7.05 (t, 1H), 6.89-6.91 (m, 2H), 4.11-4.21 (m, 2H), 3.73-3.94 (m, 5H), 2.93-3.01 (m, 6H), 2.65 (m, 3H), 2.36 (m, 2H), 2.13-2.16 (m, 2H), 1.96-2.01 (m, 2H), 1.86-1.89 (m, 2H), 1.71-1.75 (m, 1H).

### Example 44

### 7-[cis-3-(1,1-Dioxo-1-thiomorpholin-4-ylmethyl)-cyclobutyl]-5-[4-fluoro-3-(7-oxa-bicyclo[2.2.1]hept-1-ylmethoxy)-phenyl]-7H-pyrrolo[2,3-d]pyrimidin-4-ylamine

The title compound was prepared in a manner similar to Example 1 from 1-[2-fluoro-5-(4,4,5,5-tetramethyl-[1,3,2]dioxaborolan-2-yl)-phenoxymethyl]-7-oxa-bicyclo[2.2.1]heptanes (Intermediate H) and 4-((cis-3-(4-amino-5-iodo-7H-pyrrolo[2,3-d]pyrimidin-7-yl)cyclobutyl)methyl)thiomorpholine 1,1-dioxide (Intermediate AD). The crude product was purified by reverse phase preparative HPLC (Method S) to yield the title compound as a white solid. MS m/z 556.3 (M+H⁺) (Method M). ¹H-NMR (400 MHz, MeOD-d₄): b ppm 8.14 (s, 1H), 7.46 (s, 1H), 7.29 (dd, J=2.0, 8.0 Hz, 1H), 7.21 (dd, J=8.4, 11.2 Hz, 1H), 7.07 (m, 1H), 5.14 (m, 1H), 4.58 (m, 1H), 4.43 (s, 2H), 3.10 (m, 4H), 3.03 (m, 4H), 2.76 (d, J=6.8 Hz, 2H), 2.70 (m, 2H), 2.44 (m, 1H), 2.24 (m, 2H), 1.85 (m, 4H), 1.68 (m, 4H).

### Example 45

### (E)-3-(5-(4-fluoro-3-(7-oxabicyclo[2.2.1]heptan-1-ylmethoxy)phenyl)-4-amino-7H-pyrrolo[2,3-d]pyrimidin-7-yl)-1-(hydroxymethyl)cyclobutanol

The title compound was prepared in a manner similar to Example 1 starting from (E)-3-(4-amino-5-iodo-pyrrolo[2,3-d]pyrimidin-7-yl)-1-hydroxymethyl-cyclobutanol (Intermediate X) and 1-[2-Fluoro-5-(4,4,5,5-tetramethyl-[1,3,2]dioxaborolan-2-yl)-phenoxymethyl]-7-oxa-bicyclo[2.2.1]heptane (Intermediate H). Purification by reverse phase preparative HPLC (Method S) yielded the title compound as a white solid. MS m/z 455.2 (M+H⁺) (Method M).

### Example 46

### (±)-N-(cis-4-(4-amino-5-(2-fluoro-5-((tetrahydrofuran-2-yl)methoxy)phenyl)-7H-pyrrolo[2,3-d]pyrimidin-7-yl)cyclohexyl)acetamide

A mixture of (±)-7-(cis-4-aminocyclohexyl)-5-(2-fluoro-5-((tetrahydrofuran-2-yl)methoxy)phenyl)-7H-pyrrolo[2,3-d]pyrimidin-4-amine (Intermediate Z, 22 mg, 0.05 mmol), acetic acid (2 mg, 0.05 mmol), HATU (23 mg, 0.06 mmol) and DIEA (7.7 mg, 0.06 mmol) in 1.0 mL of DMF was stirred at room temperature 2 hours. The reaction mixture was partitioned between EtOAc and brine. The collected organic extracts were dried over Na₂SO₄ and evaporated. The resulting residue was purified by reverse phase preparative HPLC (Method S), yielding the title compound as a white solid. MS m/z 468.2 (M+H⁺) (Method M). ¹H-NMR (400 MHz, MeOD-d₄): δ ppm 8.16 (s, 1H), 7.44 (s, 1H), 7.17 (t, 2H), 6.99 (m, 2H), 4.69 (m, 1H), 4.25-4.29 (m, 1H), 4.16 (m, 1H), 3.83-4.07 (m, 5H), 2.08-2.15 (m, 3H), 2.03 (s, 3H), 1.88-1.94 (m, 5H), 1.78-1.86 (m, 3H).

### Example 47

### (±)-4-((cis-3-(4-amino-5-(2-fluoro-5-((tetrahydrofuran-2-yl)methoxy)phenyl)-7H-pyrrolo[2,3-d]pyrimidin-7-yl)cyclobutyl)methyl)-1-methylpiperazin-2-one

A mixture of (±)-2-[2-fluoro-5-(tetrahydro-furan-2-ylmethoxy)-phenyl]-4,4,5,5-tetramethyl-[1,3,2]dioxaborolane (Intermediate I, 35 mg, 0.109 mmol), 4-((cis-3-(4-amino-5-iodo-7H-pyrrolo[2,3-d]pyrimidin-7-yl)cyclobutyl)methyl)-1-methylpiperazin-2-one (Intermediate AA, 32 mg, 0.072 mmol), Na₂CO₃ (15 mg, 0.144 mmol), THF (2.0 ml) and water (0.5 ml) was purged with nitrogen. Tetrakis(triphenylphosphine)palladium (4.2 mg, 0.004 mmol) was then added. The reaction vessel was sealed under nitrogen and heated under microwave irradiation for 10 minutes at 120° C. The cooled reaction mixture was diluted with ethyl acetate and then sequentially washed with water and brine. The organic layers were dried over Na₂SO₄ and evaporated. The resulting residue was purified by reverse phase preparative HPLC (Method S), yielding the title compound as a white solid. MS m/z 509.3 (M+H⁺) (Method M).

### Example 48

### 5-[4-Fluoro-3-(7-oxa-bicyclo[2.2.1]hept-1-ylmethoxy)-phenyl]-7-[cis-3-((S,S)-2-oxo-2λ4-thia-5-aza-bicyclo[2.2.1]hept-5-ylmethyl)-cyclobutyl]-7H-pyrrolo[2,3-d]pyrimidin-4-ylamine

The title compound was prepared in a manner similar to Example 1 starting from (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2-oxide (Intermediate AB) and 1-[2-Fluoro-5-(4,4,5,5-tetramethyl-[1,3,2]dioxaborolan-2-yl)-phenoxymethyl]-7-oxa-bicyclo[2.2.1]heptane (Intermediate H). Purification by reverse phase preparative HPLC (Method S) yielded the title compound. MS m/z 552.2 (M+H⁺) (Method M). ¹H-NMR (400 MHz, CDCl₃): 6 ppm 11.2 (br, 2H), 8.20 (s, 1H), 7.25-7.23 (m, 2H), 7.13 (s, 1H), 6.96-6.93 (m, 1H), 5.14-5.05 (m, 1H), 4.58 (t, 1H), 4.38 (s, 2H), 3.55-3.52 (m, 2H), 2.70-2.65 (m, 2H), 2.50-2.38 (m, 4H), 2.23-2.10 (m, 3H), 2.14-2.05 (m, 2H), 1.87-1.76 (m, 4H), 1.63-1.58 (m, 4H).

### Example 49

### 5-[2-Fluoro-5-(tetrahydro-furan-2-ylmethoxy)-phenyl]-7-[cis-3-((S,S)-2-oxo-2λ4-thia-5-aza-bicyclo[2.2.1]hept-5-ylmethyl)-cyclobutyl]-7H-pyrrolo[2,3-d]pyrimidin-4-ylamine

The title compound was prepared in a manner similar to Example 1 from (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2-oxide (Intermediate AB) and 2-[2-Fluoro-5-(tetrahydro-furan-2-ylmethoxy)-phenyl]-4,4,5,5-tetramethyl-[1,3,2]dioxaborolane (Intermediate I). Purification by reverse phase preparative HPLC (Method S) yielded the title compound. MS m/z 526.2 (M+H⁺) (Method M).

### Example 50

### 5-[4-Fluoro-3-(tetrahydro-furan-2-ylmethoxy)-phenyl]-7-[cis-3-((S,S)-2-oxo-2λ4-thia-5-aza-bicyclo[2.2.1]hept-5-ylmethyl)-cyclobutyl]-7H-pyrrolo[2,3-d]pyrimidin-4-ylamine

The title compound was prepared in a manner similar to Example 1 from (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2-oxide (Intermediate AB) and 2-[4-Fluoro-3-(tetrahydro-furan-2-ylmethoxy)-phenyl]-4,4,5,5-tetramethyl-[1,3,2]dioxaborolane (Intermediate G). Purification by reverse phase preparative HPLC (Method S) yielded the title compound. MS m/z 526.2 (M+H⁺) (Method M).

### Example 51

### 4-(cis-3-{4-Amino-5-[4-fluoro-3-(7-oxa-bicyclo[2.2.1]hept-1-ylmethoxy)-phenyl]-pyrrolo[2,3-d]pyrimidin-7-yl}-cyclobutylmethyl)-1-methyl-piperazin-2-one

The title compound was prepared in a manner similar to Example 1 from 4-((cis-3-(4-amino-5-iodo-7H-pyrrolo[2,3-d]pyrimidin-7-yl)cyclobutyl)methyl)-1-methylpiperazin-2-one (Intermediate AA) and 1-[2-Fluoro-5-(4,4,5,5-tetramethyl-[1,3,2]dioxaborolan-2-yl)-phenoxymethyl]-7-oxa-bicyclo[2.2.1]heptane (Intermediate H). Purification by reverse phase preparative HPLC (Method S) yielded the title compound. MS m/z 535.3 (M+H⁺) (Method M). ¹H-NMR (400 MHz, MeOD-d₄, TFA salt): δ ppm 8.34 (s, 1H), 7.75 (s, 1H), 7.34 (dd, J=2.0, 8.0 hz, 1H), 7.26 (dd, J=8.4, 11.2 Hz, 1H), 7.09 (m, 1H), 5.33 (m, 1H), 4.58 (m, 1H), 4.45 (s, 2H), 3.91 (s, 2H), 3.67 (m, J=5.4 Hz, 2H), 3.59 (t, J=5.2 Hz, 2H), 3.47 (d, J=6.8 Hz, 2H), 3.03 (s, 3H), 2.87 (m, 2H), 2.74 (m, 1H), 2.54 (m, 2H), 1.85 (4H), 1.69 (m, 4H).

### Example 52

### 5-[4-Fluoro-3-(7-oxa-bicyclo[2.2.1]hept-1-ylmethoxy)-phenyl]-7-[cis-3-(1-oxo-1λ4-thiomorpholin-4-ylmethyl)-cyclobutyl]-7H-pyrrolo[2,3-d]pyrimidin-4-ylamine

The title compound was prepared in a manner similar to Example 1 from 5-iodo-7-[cis-3-(1-oxo-thiomorpholin-4-ylmethyl)-cyclobutyl]-7H-pyrrolo[2,3-d]pyrimidin-4-ylamine (Intermediate Q) and 1-[2-Fluoro-5-(4,4,5,5-tetramethyl-[1,3,2]dioxaborolan-2-yl)-phenoxymethyl]-7-oxa-bicyclo[2.2.1]heptane (Intermediate H). Purification by reverse phase preparative HPLC (Method S) yielded the title compound. MS m/z 540.2 (M+H+) (Method M).

### Example 53

### 7-[cis-3-((S,S)-2,2-Dioxo-2λ6-thia-5-aza-bicyclo[2.2.1]hept-5-ylmethyl)-cyclobutyl]-5-[4-fluoro-3-(7-oxa-bicyclo[2.2.1]hept-1-ylmethoxy)-phenyl]-7H-pyrrolo[2,3-d]pyrimidin-4-ylamine

The title compound was prepared in a manner similar to Example 1 from (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2,2-dioxide (Intermediate AC) and 1-[2-Fluoro-5-(4,4,5,5-tetramethyl-[1,3,2]dioxaborolan-2-yl)-phenoxymethyl]-7-oxa-bicyclo[2.2.1]heptane (Intermediate H). Purification by reverse phase preparative HPLC (Method S) yielded the title compound. MS m/z 568.2 (M+H⁺) (Method M). ¹H-NMR (400 MHz, MeOD-d₄): 6 ppm 8.13 (s, 1H), 7.46 (s, 1H), 7.30 (dd, J=2.0, 8.0 Hz, 1H), 7.20 (dd, J=8.0, 11.2 Hz, 1H), 7.02-6.95 (m, 2H), 5.15 (m, 1H), 4.58 (m, 1H), 4.43 (s, 2H), 3.80 (t, J=3.2 Hz, 1H), 3.57 (m, 2H), 3.38 (m, 3H), 3.27 (d, J=11.2 Hz, 1H), 3.13 (dd, J=4.4 Hz, 1H), 2.95 (dd, J=3.6, 12.8 Hz, 1H), 2.83 (m, 2H), 2.77 (m, 2H), 2.46-2.30 (m, 3H), 2.45 (m, 2H), 1.84 (m, 4H), 1.67 (m, 4H).

### Example 54

### 7-[cis-3-((S,S)-2,2-Dioxo-2λ6-thia-5-aza-bicyclo[2.2.1]hept-5-ylmethyl)-cyclobutyl]-5-[2-fluoro-5-(tetrahydro-furan-2-ylmethoxy)-phenyl]-7H-pyrrolo[2,3-d]pyrimidin-4-ylamine

The title compound was prepared in a manner similar to Example 1 from (1S,4S)-5-((cis-3-(4-amino-5-iodo-7H-pyrrolo[2,3-d]pyrimidin-7-yl)cyclobutyl)methyl)-2-thia-5-azabicyclo[2.2.1]heptane 2,2-dioxide (Intermediate AC) and 2-[2-Fluoro-5-(tetrahydro-furan-2-ylmethoxy)-phenyl]-4,4,5,5-tetramethyl-[1,3,2]dioxaborolane (Intermediate I). Purification by reverse phase preparative HPLC (Method S) yielded the title compound. MS m/z 542.2 (M+H⁺) (Method M).

### Example 55

### (±)-5-[2-Fluoro-5-(tetrahydro-furan-2-ylmethoxy)-phenyl]-7-[cis-3-(1-oxo-1λ4-thiomorpholin-4-ylmethyl)-cyclobutyl]-7H-pyrrolo[2,3-d]pyrimidin-4-ylamine

The title compound was prepared in a manner similar to Example 1 from 5-iodo-7-[cis-3-(1-oxo-thiomorpholin-4-ylmethyl)-cyclobutyl]-7H-pyrrolo[2,3-d]pyrimidin-4-ylamine (Intermediate Q) and 2-[2-fluoro-5-(tetrahydro-furan-2-ylmethoxy)-phenyl]-4,4,5,5-tetramethyl-[1,3,2]dioxaborolane (Intermediate I). Purification by reverse phase preparative HPLC (Method S) yielded the title compound. MS m/z 514.2 (M+H⁺) (Method M).

### Example 56

### (±)—N-(3-cis-{4-Amino-5-[2-fluoro-5-(tetrahydro-furan-2-ylmethoxy)-phenyl]-pyrrolo[2,3-d]pyrimidin-7-yl}-cyclobutylmethyl)-acetamide

The title compound was prepared in a manner similar to Example 1 from N-((cis-3-(4-amino-5-iodo-7H-pyrrolo[2,3-d]pyrimidin-7-yl)cyclobutyl)methyl)acetamide (Intermediate AE) and 2-[2-Fluoro-5-(tetrahydro-furan-2-ylmethoxy)-phenyl]-4,4,5,5-tetramethyl-[1,3,2]dioxaborolane (Intermediate I). Purification by reverse phase preparative HPLC (Method S) yielded the title compound. MS m/z 454.2 (M+H⁺) (Method M). ¹H-NMR (400 MHz, MeOD-d₄): δ ppm 8.14 (s, 1H), 7.45 (s, 1H), 7.16 (t, J=9.2 Hz, 1H), 7.02-6.95 (m, 2H), 5.09 (m, 1H), 4.26 (m, 1H), 4.04 (dd, J=3.6, 10.0 Hz, 1H), 3.97 (dd, J=6.4, 10.0 Hz, 1H), 3.90 (m, 1H), 3.81 (m, 1H), 3.33 (m, 2H), 2.65 (m, 2H), 2.39 (m, 1H), 2.26 (m, 2H), 2.09 (m, 1H), 1.97 (s, 3H), 1.95 (m, 2H), 1.79 (m, 1H).

### Example 57 and 58

### 3-[cis-3-((S,S)-2,2-dioxo-2λ6-thia-5-aza-bicyclo[2,2,1]hept-ylmethyl)-cyclobutyl]-5-[2-fluoro-5-(tetrahydro-furan-(2S)-ylmethoxyl)phenyl]-7H-pyrrolo[2,3-d]pyrimidine-4-ylamine and 3-[cis-3-((S,S)-2,2-dioxo-2λ6-thia-5-aza-bicyclo[2,2,1]hept-ylmethyl)-cyclobutyl]-5-[2-fluoro-5-(tetrahydro-furan-(2R)-ylmethoxyl)phenyl]-7H-pyrrolo[2,3-d]pyrimidine-4-ylamine

7-[cis-3-((S,S)-2,2-Dioxo-2λ6-thia-5-aza-bicyclo[2.2.1]hept-5-ylmethyl)-cyclobutyl]-5-[2-fluoro-5-(tetrahydro-furan-2-ylmethoxy)-phenyl]-7H-pyrrolo[2,3-d]pyrimidin-4-ylamine (Example 54) was separated into pure optical isomers via chiral Prepratory HPLC (Column: 20×250 mm ChiralPak IA; Conditions: 24 mL/min flow rate, 60/30/10 Hexane/CHCl₃/EtOH with 0.1% DEA as modifier Run Time: 15 minutes). Analytical chiral HPLC retention times: 9.72 min. (Example 57) and 11.60 min (Example 58) under these analytical chiral HPLC conditions: Column: 4.6×250 mm ChiralPak IA; Conditions: 1 mL/min flow rate, 60/30/10 Hexane/CHCl₃/EtOH. Example 57 as the first eluting isomer. MS m/z 542.3 (M+H⁺) (Method M). Example 58 as the second eluting isomer. MS m/z 542.3 (M+H⁺) (Method M). NMR data were identical for Example 57 and 58. ¹H-NMR (400 MHz, CDCl₃): δ ppm 8.28 (s, 1H), 7.17 (s, 1H), 7.08 (d, 1H), 6.93 (dd, 1H), 6.88-6.84 (m, 1H), 5.18-5.12 (m, 2H), 4.28-4.22 (m 1H), 3.98-3.91 (m, 3H), 3.84-3.78 (m, 1H), 3.72-3.66 (m, 1H), 3.47 (br, 1H), 3.36 (dd, 1H), 3.26 (d, 1H), 3.16 (dd, 1H), 2.89-2.83 (m, 2H), 2.75-2.67 (m, 2H), 2.48 (d, 1H), 2.38-2.35 (m, 1H), 2.32-2.25 (m, 1H) 2.25-2.01 (m 3H), 1.96-1.88 (m, 2H), 1.77-1.68 (m, 1H).

In another aspect, the invention provides a process of manufacturing a compound of formula (I), or a pharmaceutically acceptable salt and/or solvate thereof, as described herein.

In the following table, the substituents listed link to the core bicyclic heterocycle to form a compound of formula (I). For example, the compound of Example 1 has the structure below, as also described hereinabove.

### Example 1

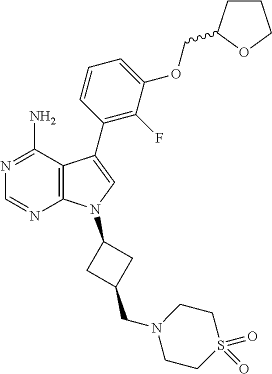 [SMILES: Nc1ncnc2c1c(-c1cccc(OCC3CCCO3)c1F)cn2[C@H]1C[C@@H](CN2CCS(=O)(=O)CC2)C1]

| Ex. | 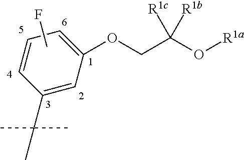 | 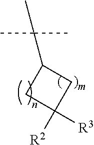 | IGF-1R IC₅₀ (nM) |
|---|---|---|---|
| 1 | 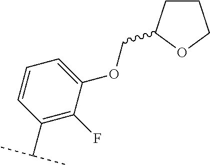 | 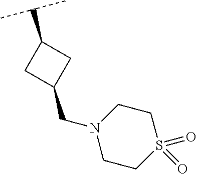 | 5 |
| 2 | 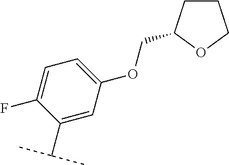 | 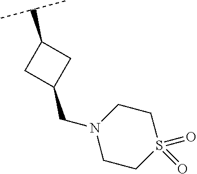 | 198 |
| 3 | 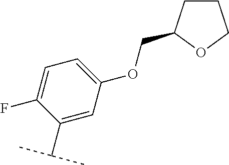 | 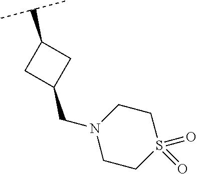 | — |
| 4 | 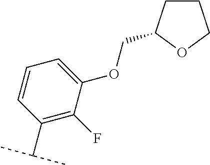 | 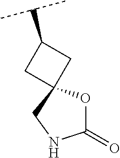 | 17 |
| 5 | 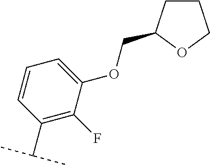 | 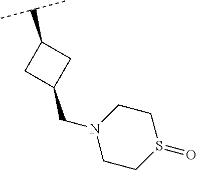 | 16 |
| 6 | 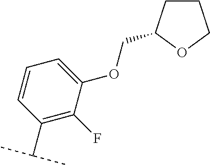 | 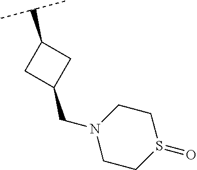 | 10 |
| 7 | blank | | |
| 8 | 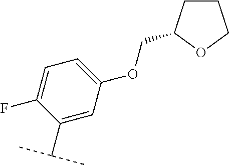 | 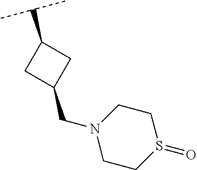 | 14 |
| 9 | 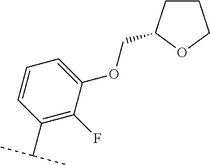 | 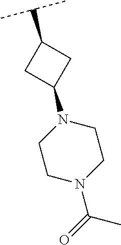 | 16 |
| 10 | 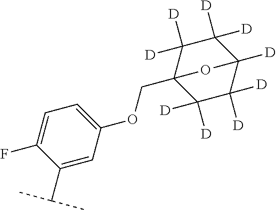 | 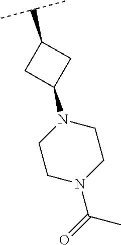 | 12 |
| 11 | 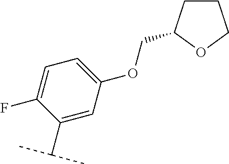 | 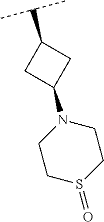 | 92 |
| 12 | 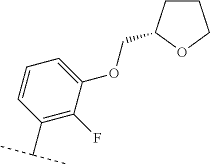 | 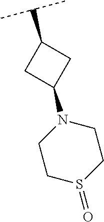 | 25 |
| 13 | 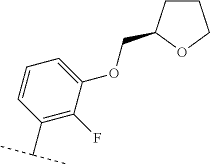 | 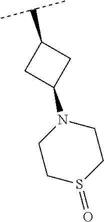 | 42 |
| 14 | 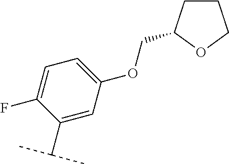 | 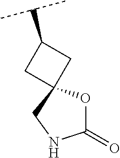 | 52 |
| 15 | 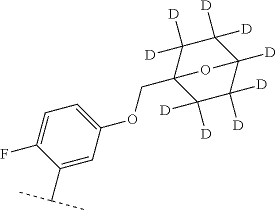 | 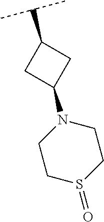 | 10 |
| 16 | 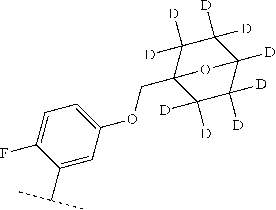 | 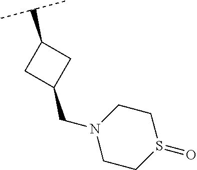 | 9 |
| 17 | 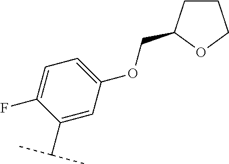 | 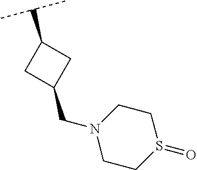 | 93 |
| 18 | 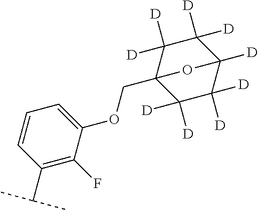 | 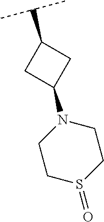 | 4 |
| 19 | 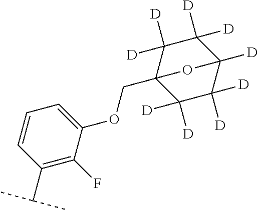 | 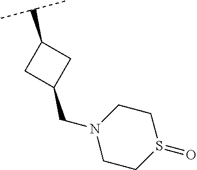 | 2 |
| 20 | 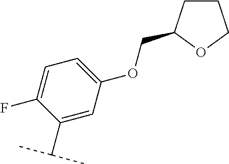 | 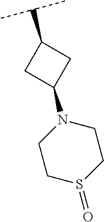 | 198 |
| 21 | 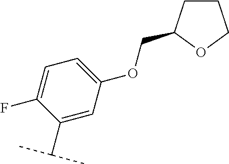 | 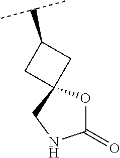 | 215 |
| 22 | 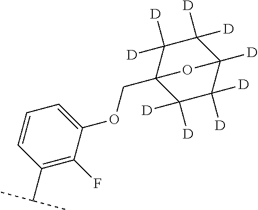 | 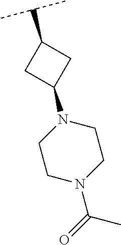 | 3 |
| 23 | 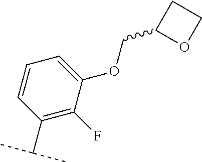 | 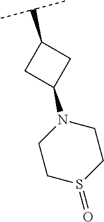 | 452 |
| 24 | 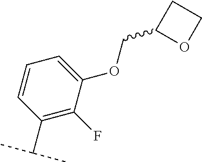 | 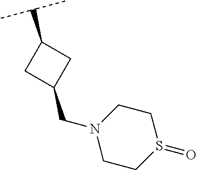 | 368 |
| 25 | 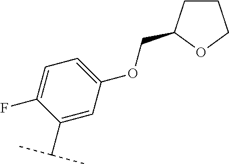 | 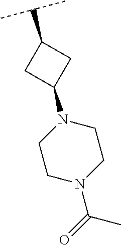 | 159 |
| 26 | 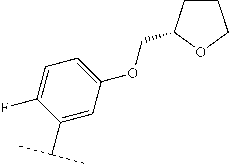 | 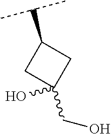 | — |
| 27 | 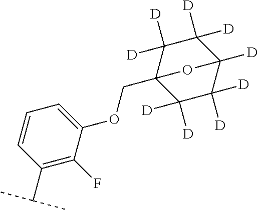 | 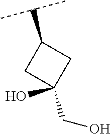 | — |
| 28 | 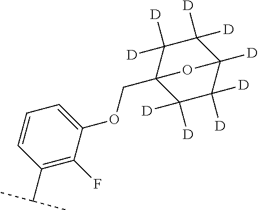 | 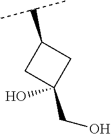 | — |
| 29 | 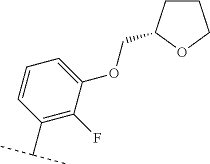 | 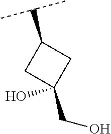 | — |
| 30 | 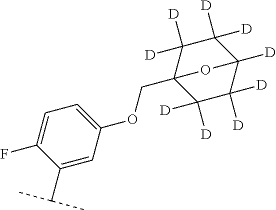 | 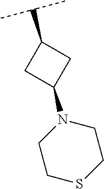 | — |
| 31 | 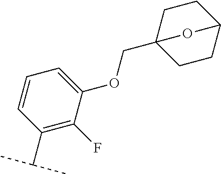 | 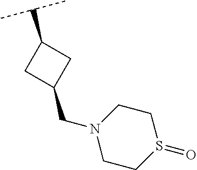 | — |
| 32 | 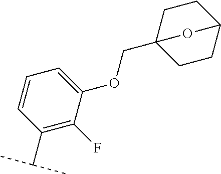 | 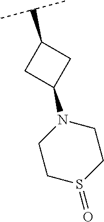 | — |
| 33 | 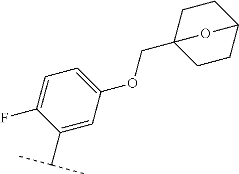 | 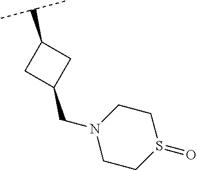 | — |
| 34 | 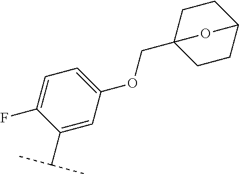 | 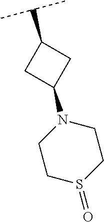 | — |
| 35 | 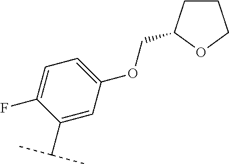 | 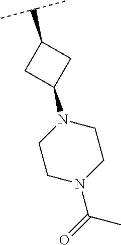 | 13 |
| 36 | 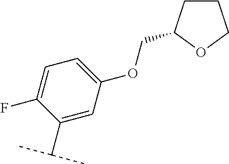 | 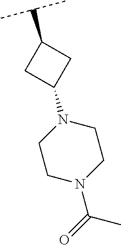 | — |
| 37 | 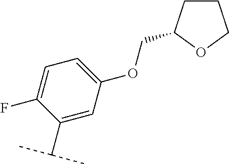 | 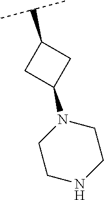 | — |
| 38 | 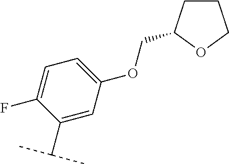 | 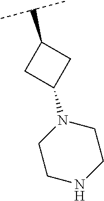 | — |
| 39 | 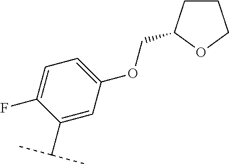 | 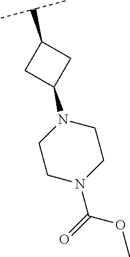 | — |
| 40 | 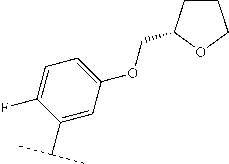 | 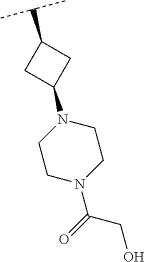 | — |
| 41 | 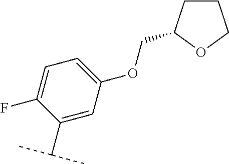 | 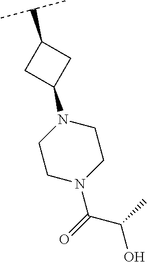 | — |
| 42 | 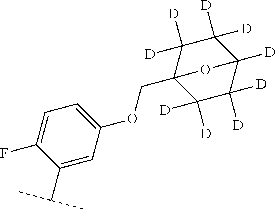 | 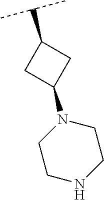 | — |
| 43 | 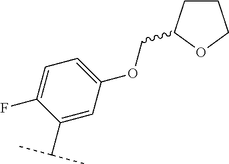 | 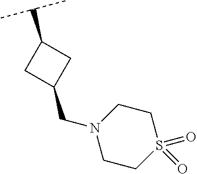 | 19 |
| 44 | 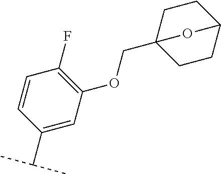 | 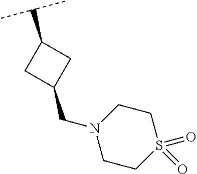 | 18 |
| 45 | 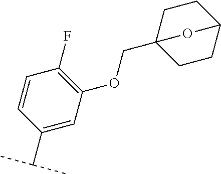 | 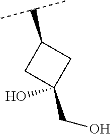 | 461 |
| 46 | 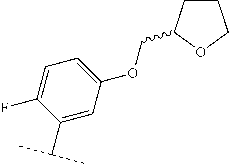 | 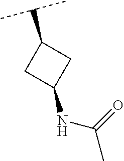 | — |
| 47 | 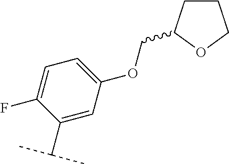 | 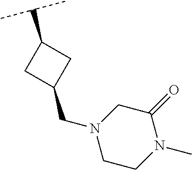 | — |
| 48 | 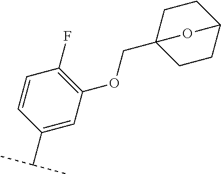 | 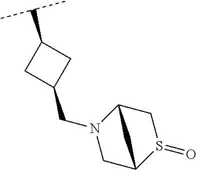 | 16-98 |
| 49 | 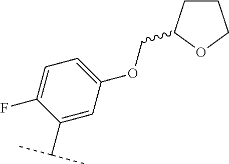 | 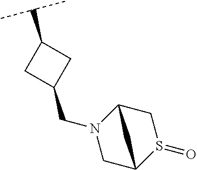 | — |
| 50 | 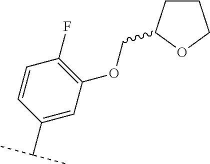 | 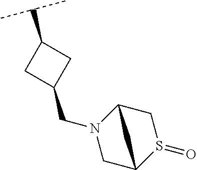 | — |
| 51 | 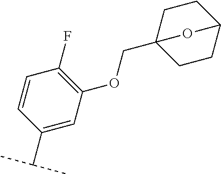 | 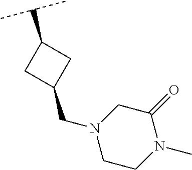 | 110 |
| 52 | 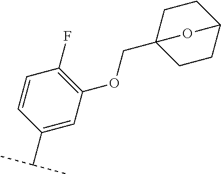 | 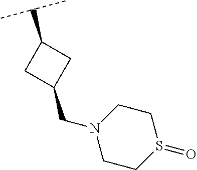 | 31 |
| 53 | 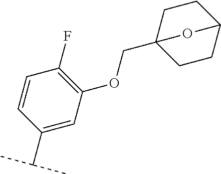 | 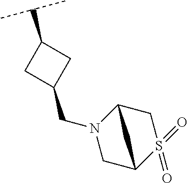 | 22 |
| 54 | 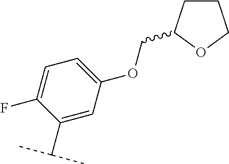 | 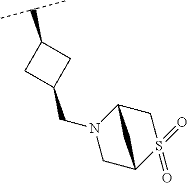 | 31 |
| 55 | 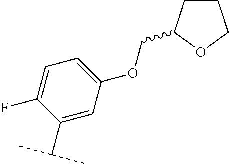 | 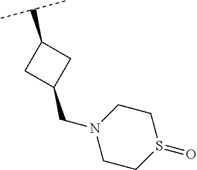 | 136 |
| 56 | 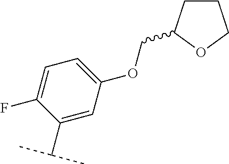 | 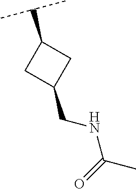 | 95 |
| 57 | 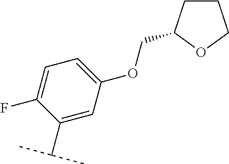 | 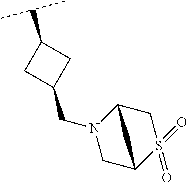 | — |
| 58 | 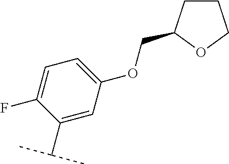 | 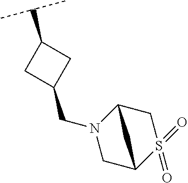 | — |

1. A compound of formula (I): 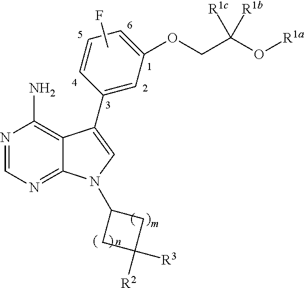
or a pharmaceutically acceptable salt and/or solvate thereof,
wherein
F is fluoro, said fluoro being substituted in the 2, 4 or 6 position of the phenyl ring;
R^{1a} and R^{1b} together with the carbon and oxygen atoms to which they are attached, form a fully saturated ring comprising 2, 3 or 4 further carbon ring atoms, optionally wherein one, several, or all of the hydrogen atoms attached to the carbon ring atoms are replaced with deuterium, and wherein said saturated ring is optionally substituted by 1 or 2 methyl substituents, and R^{1c} is H,
or
R^{1a}, R^{1b} and R^{1c}, together with the atoms to which they are attached, form a fully saturated bridged ring comprising 5 further ring carbon atoms, optionally wherein one, several, or all of the hydrogen atoms attached to the carbon ring atoms are replaced with deuterium;
n and m are both 1, or n and m are both 2;
R² is H or OH,
R³ is selected from:
—CH₂₍ₚ₎₋heterocyclic², wherein said heterocyclic² is a 4, 5, 6, 7, 8 or 9 membered saturated, unsaturated or partially saturated ring or ring system comprising carbon ring atoms and 1, 2, 3 or 4 ring heteroatoms independently selected from N, O and S, wherein the total number of ring S atoms does not exceed 1, and the total number of ring O atoms does not exceed 1, and said heterocyclic² is optionally substituted with 1, 2, 3 or 4 substituents independently selected from oxo, —C(═O)—C₁-C₄alkyl, —C(═O)—O—C₁-C₄alkyl, C₁-C₄alkyl, fluoro, —C(═O)—NR⁴R⁵ and hydroxy,
wherein said —C(═O)—C₁-C₄alkyl and C₁-C₄alkyl are each optionally substituted by 1 or 2 hydroxy substituents and/or 1, 2 or 3 fluoro substituents,
—CH₂₍ₚ₎—N(R⁴)—C(═O)—C₁-C₄alkyl,
—CH₂₍ₚ₎—N(R⁴)—C(═O)—O—C₁-C₄alkyl,
—CH₂₍ₚ₎—N(R⁴)—C(═O)—O—C₁-C₆cycloalkyl,
—CH₂₍ₚ₎—N(R⁴)—C₁-C₆cycloalkyl,
—CH₂—OH,
—CH₂₍ₚ₎—NR⁴R⁵
—CH₂₍ₚ₎—N(R⁴)—C(═O)—NR⁴—C₁-C₄alkyl,
wherein said C₁-C₄alkyl and C₁-C₆cycloalkyl of —CH₂₍ₚ₎—N(R⁴)—C(═O)—C₁-C₄alkyl, —CH₂₍ₚ₎—N(R⁴)—C(═O)—O—C₁-C₆cycloalkyl, and —CH₂₍ₚ₎—N(R⁴)—C₁-C₆cycloalkyl are each optionally substituted by 1, 2, 3 or 4 substituents independently selected from fluoro, methyl and trifluoromethyl,
or R² and R³, together with the carbon to which they are attached, form a 5 membered saturated heterocyclic ring containing carbon ring atoms and either:
1 or 2 ring N atoms,
1 ring N atom and a ring O atom, or
2 ring O atoms,
wherein said heterocyclic ring is substituted at a ring carbon atom by an oxo substituent;
p is 0 or 1;
R⁴ and R⁵ are each independently selected from H and C₁-C₄alkyl, wherein said C₁-C₄alkyl is optionally substituted with 1, 2 or 3 substituents independently selected from halo and hydroxy.

2. A compound of formula (I) as claimed in claim 1, or a pharmaceutically acceptable salt and/or solvate thereof, wherein
R^{1a} and R^{1b} together with the carbon and oxygen atoms to which they are attached, form a fully saturated ring comprising 2, 3 or 4 further carbon ring atoms, optionally wherein one, several, or all of the hydrogen atoms attached to the carbon ring atoms are replaced with deuterium, and R^{1c} is H,
or
R^{1a}, R^{1b} and R^{1c}, together with the atoms to which they are attached, form a fully saturated bridged ring comprising 5 further ring carbon atoms, optionally wherein one, several, or all of the hydrogen atoms attached to the carbon ring atoms are replaced with deuterium;
R³ is selected from:
—CH₂₍ₚ₎₋heterocyclic², wherein said heterocyclic² is a 6, 7 or 8 membered saturated ring or ring system comprising carbon ring atoms and 1, 2 or 3 ring heteroatoms independently selected from N, O and S, wherein the total number of ring S atoms does not exceed 1, and the total number of ring O atoms does not exceed 1, and said heterocyclic² is optionally substituted with 1, 2, 3 or 4 substituents independently selected from oxo, —C(═O)—C₁-C₄alkyl, —C(═O)—O—C₁-C₄alkyl, C₁-C₄alkyl, wherein said —C(═O)—C₁-C₄alkyl is optionally substituted by 1 or 2 hydroxy substituents,
—CH₂₍ₚ₎—N(R⁴)—C(═O)—C₁-C₄alkyl,
—CH₂—OH,
or R² and R³, together with the carbon to which they are attached, form a 5 membered saturated heterocyclic ring containing carbon ring atoms and either:
1 or 2 ring N atoms,
1 ring N atom and a ring O atom, or
2 ring O atoms,
wherein said heterocyclic ring is substituted at a ring carbon atom by an oxo substituent;
p is 0 or 1;
and
R⁴ is selected from H and C₁-C₄alkyl.

3. A compound of formula (I) as described in claim 1, or a pharmaceutically acceptable salt and/or solvate thereof, wherein F is fluoro, said fluoro being substituted in the 2 or 4 position of the phenyl ring.

4. A compound of formula (I) as described in claim 1, or a pharmaceutically acceptable salt and/or solvate thereof, wherein R^{1a} and R^{1b} together with the carbon and oxygen atoms to which they are attached, form tetrahydrofuranyl, and R^{1c} is H.

5. A compound of formula (I) as described in claim 1, or a pharmaceutically acceptable salt and/or solvate thereof, wherein R^{1a}, R^{1b} and R^{1c}, together with the atoms to which they are attached, form an optionally deuterated 7-oxa-bicyclo[2.2.1]heptan-1-yl ring system.

6. A compound of formula (I) as described in claim 1, or a pharmaceutically acceptable salt and/or solvate thereof, wherein n and m are both 1.

7. A compound of formula (I) as described in claim 1, or a pharmaceutically acceptable salt and/or solvate thereof, wherein R³ is
—CH₂₍ₚ₎₋heterocyclic², wherein said heterocyclic² is thiomorpholinyl, piperazinyl or thia-aza-bicyclo[2.2.1]hepantyl, each thiomorpholinyl, piperazinyl or thia-aza-bicyclo[2.2.1]heptanyl being optionally substituted with 1 or 2 substituents independently selected from oxo, —C(═O)—C₁-C₂alkyl, and —C(═O)—O—C₁-C₂alkyl, wherein said —C(═O)—C₁-C₂alkyl is optionally substituted with one hydroxy,
hydroxymethyl-, or
—CH₂₍ₚ₎—NH—C(═O)—C₁-C₂alkyl.

8. A compound of formula (I) as described in claim 1, or a pharmaceutically acceptable salt and/or solvate thereof, wherein R² and R³, together with the carbon to which they are attached, form 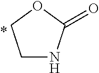
wherein * indicates the point of attachment.

9. A compound of formula (I), or a pharmaceutically acceptable salt and/or solvate thereof, which is disclosed as an Example herein.

10. A pharmaceutical composition comprising a compound, or a pharmaceutically acceptable salt and/or solvate thereof, according to claim 1, and one or more pharmaceutically acceptable carriers.

11. A combination comprising a compound, or a pharmaceutically acceptable salt and/or solvate thereof, according to claim 1, and one or more therapeutically active co-agents.

12. A method of treating a disorder or a disease in a subject mediated by IGF-1R, wherein the method comprises administering to the subject a therapeutically effective amount of the compound of formula (I), or a pharmaceutically acceptable salt and/or solvate thereof, according to claim 1.

13-14. (canceled)
